(19) United States
(12) Patent Application Publication
(10) Pub. No.: US 2006/0079439 A1
(43) Pub. Date: Apr. 13, 2006

(54) Elastin prevents occlusion of body vessels by vascular smooth muscle cells

(75) Inventors: Dean Y Li, Salt Lake City, UT (US); Satyajit Karnik, Fremont, CA (US)

Correspondence Address: FISH & NEAVE IP GROUP;ROPES & GRAY LLP, ONE INTERNATIONAL PLACE, BOSTON, MA, 02110-2624, US

(73) Assignee: University of Utah Research Foundation, Salt Lake City, UT (US)

(21) Appl. No.: 10/509,472

(22) PCT Filed: Mar. 27, 2003
(86) PCT No.: PCT/US03/09391

§ 371 Date: Sep. 21, 2005

Related U.S. Application Data
(60) Provisional application No. 60/368,084, filed on Mar. 27, 2002.

(51) Int. Cl. A61K 48/00; A61K 38/17; A61K 31/4745; A61K 31/337; C40B 40/10

(52) U.S. Cl. 514/2; 514/44; 514/291; 514/449; 435/7.1

(57) ABSTRACT

The present invention provides methods and compositions for promoting elastin signaling in smooth muscle cells including vascular smooth muscle cell. The present invention further provides methods of identifying additional agents that promote elasfin signaling.

### RELATED APPLICATIONS

This application claims priority to U.S. provisional application 60/368,084 filed Mar. 27, 2002, the disclosure of which is hereby incorporated by reference in its entirety.

### BACKGROUND OF THE INVENTION

Supravalvular aortic stenosis (SVAS) is an inherited obstructive vascular disorder that causes hemodynamically significant narrowing of arteries. Although the aorta is most frequently diseased, any artery can be affected, including the pulmonary, carotid and coronary arteries. The onset and severity of vascular disease in SVAS is variable. In general a pediatric population without common risk factors such as smoking, high serum cholesterol, and high blood pressure is most affected. The pathology of SVAS arteries includes hypertrophy and hyperplasia of vascular smooth muscle cells, disrupted and disorganized elastic fibers, abundant deposition of matrix components like collagen, and inflammatory cells. These findings are commonly observed not only in SVAS but also in many other obstructive vascular diseases including atherosclerosis and coronary restenosis. If untreated, SVAS and other obstructive vascular diseases can lead to heart failure, myocardial infarction, stroke and death.

Four lines of evidence support a role for the elastin gene, ELN, in SVAS. First, ELN is genetically linked to the disease's phenotype in autosomal dominant SVAS. Second, large rearrangements and point mutations that disrupt ELN are associated with the disease in SVAS families. Third, de novo point mutations in ELN are associated with sporadic cases of SVAS. Elastin is the dominant arterial extracellular matrix protein comprising 50% of the dry weight of some arteries. Elastin is encoded by a single gene on human chromosome 7q11.23, synthesized by smooth muscle, secreted as the monomer tropoelastin, and organized into polymers that form concentric rings of elastic lamellae around the arterial lumen. Each elastic lamella alternates with a ring of smooth muscle. These lamellae provide the resilience that arteries need to absorb hemodynamic stress of cardiac systole and to release this energy in the form of sustained blood pressure during diastole. Thus, similar to other matrix proteins, elastin has well characterized roles in providing structural support to the vessel wall.

Some diseases of the vasculature are characterized by vascular disintegration and dissection, and these diseases highlight the importance of maintaining the structural integrity of vessel walls. A second cause of diseases of the vasculature are conditions which result in vascular obstruction. Without being bound by theory, mutations in elastin and/or disruptions in elastin signaling are implicated in obstructive vascular diseases. For example, mutations in the elastin gene cause vascular obstruction in human disease and animal models, not aneurysms or dissections. In gene-targeting experiments, disruption of the elastin gene is sufficient to cause an obstructive vascular pathology characterized by excessive subendothelial proliferation and accumulation of vascular smooth muscle cells that occlude arteries. This obstructive vascular pathology in Eln −/− arteries is reproduced in organ culture in the absence of hemodynamic stress and is reminiscent of the pathology observed in coronary restenosis (FIG. 2). We concluded from these studies that elastin is a molecular determinant of late arterial morphogenesis, stabilizing arterial structure by regulating proliferation and organization of vascular smooth muscle cells.

In contrast to skeletal and cardiac muscles, vascular smooth muscle cells (vsmcs) can alternate between a quiescent contractile state and a proliferative non-contractile state (Schmidt et al., 1969; O'Connor et al., 1985). Vascular smooth muscle cells in a mature artery are quiescent and largely comprised of contractile apparatus that function to dilate and constrict the lumen as required by physiologic demands. Actin stress fibers serve as the scaffold for the contractile apparatus and are a hallmark of mature vsmcs. Under circumstances of injury, repair and regeneration, vascular smooth muscle cells lose their contractile apparatus and dedifferentiate into a synthetic phenotype capable of proliferating and secreting matrix elements. As synthetic vsmcs reengage their program to proliferate and secrete matrix proteins, there is loss of actin stress fibers and an increase in the golgi and endoplasmic reticulum. Accordingly, the phenotypic modulation of vsmcs offers a tempting target for preventing obstructive vascular disease.

Obstructive vascular diseases such as atherosclerosis and vascular restenosis cause almost 50% of the mortality in the United States. Obstructive pathology consists of an accumulation of vsmcs and extracellular matrix in the subendothelial space that occludes arterial lumens and restricts blood flow. Over the last 10-20 years a central model explaining the pathogenesis of obstructive vascular diseases has emerged. This model has been named the response-to-injury model. Though advances in our understanding of the molecular and cellular events involved have been made, the fundamental tenets of this model as enunciated and championed by Russell Ross remain unchanged. First, injury to the vessel wall incites inflammation. Second, macrophages and monocytes release growth factors and cytokines including platelet-derived growth factor. Third, some of these paracrine factors induce vascular smooth muscle cells to dedifferentiate from a quiescent contractile phenotype to a proliferative non-contractile phenotype. These vsmcs proliferate and accumulate within the subendothelial space eventually occluding the arterial lumen. Strategies to reverse obstructive vascular disease by reducing inflammation or blocking the activity of secreted paracrine factors are suggested by this model. Though these strategies have succeeded in culture, they have not proven to be effective in animal models or clinical settings.

Medical therapies focus on reducing the risk factors associated with obstructive vascular disease. Anti-thrombotic, anti-hypertensive, and cholesterol-lowering medications are aimed at decreasing the risk of occlusion, while beta-blockers and angiotensin-converting enzyme inhibitors act by reducing the workload of the heart. Despite these pharmaceutical advances that reduce the risk of vascular occlusion and cardiac events, the need for interventional cardiology and cardiac surgery to directly treat vascular obstructions remains immense.

Angioplasty is the major intervention for coronary artery disease accounting for over 680,000 procedures annually in the United States alone. Briefly, a balloon is placed within a blocked artery and expanded to relieve the obstruction. In most cases, a metal stent is placed within the instrumented artery. However, only 70% of angioplasties lead to long-term (6 months) relief of arterial obstruction. In a process termed restenosis, vsmcs dedifferentiate, proliferate, and reocclude the artery in response to the vascular injury caused by angioplasty. Such restenosis is also observed in other procedures which cause injury to a body vessel wall including the placement of stents, wires, catheters, and other intraluminal devices. Continued advances in the geometry and composition of stents have largely impacted ease of stent delivery, but have not lessened the complication of restenosis. Recently, strategies employing radioactivity and other cytotoxic agents (e.g. paclitaxil and rapamycin) to treat restenosis have received substantial attention. These strategies rely on the temporary and local delivery of toxic agents that block proliferation of many cell types. The long-term efficacy is currently being tested, however given the toxicity of each agent it is doubtful that they can be used in combination with one another. The biologic effect of elastin is not based on cellular toxicity and has the potential to be used in isolation or in combination with other agents. Importantly, manipulating elastin signaling systemically can be considered, in contrast to radioactivity, rapamycin, actinomycin D or paclitaxil.

Unlike skeletal and cardiac muscles, vascular smooth muscle cells enjoy tremendous plasticity and are able to transform themselves from a mature differentiated state to a proliferative dedifferentiated state. This cellular plasticity is required because it enables the vascular system to regenerate and grow. However, plasticity must be balanced by the need to maintain a mature and stable structure capable of circulating blood throughout a whole animal. Because vsmcs modulate their phenotype readily, external factors must instruct them to remain in a mature state if homeostasis is to be achieved.

In our model, disruption of elastin signaling significantly contributes to the pathogenesis of obstructive vascular diseases FIG. 3). If this model is correct, then the opportunity exists to prevent or reduce such diseases using agents which promote elastin signaling. Given the tremendous impact of obstructive vascular diseases on the duration and quality of life of their sufferers, there exists a need to develop methods and compositions for the treatment and prevention of disorders that result in the occlusion of body vessels. The present invention provides such methods and compositions.

### SUMMARY OF THE INVENTION

The invention provides compositions and methods for modulating the proliferation, differentiation, cellular, and morphological properties of vascular smooth muscle cells. The invention provides compositions and methods for treating and preventing obstructive vascular diseases including genetic vascular diseases, as well as vascular diseases and conditions caused by injury, age, behavioral, and other factors which may or may not have a genetic component. The invention further provides compositions and methods for treating and preventing obstructive of a body vessel, whether the obstruction is caused by injury or disease.

In a first aspect, the invention provides a method of decreasing or preventing occlusion of a body vessel by smooth muscle cells, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a second aspect, the invention provides a method of decreasing vascular obstruction, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a third aspect, the invention provides a method of promoting actin stress fiber formation in a smooth muscle cell, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a fourth aspect, the invention provides a method of promoting actin polymerization in a smooth muscle cell, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a fifth aspect, the invention provides a method of increasing the ratio of F:G actin in a smooth muscle cell, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct. In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a sixth aspect, the invention provides a method of promoting focal adhesion formation in a smooth muscle cell, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a seventh aspect, the invention provides a method of treating or prophylactically treating an obstructive vascular disease, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In an eighth aspect, the invention provides a method of prophylactically treating stenosis, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a ninth aspect, the invention provides a method of treating or prophylactically treating restenosis, comprising administering an agent that promotes elastin signaling.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct. % In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In a tenth aspect, the invention provides use of an agent the promotes elastin signaling in smooth muscle cells for the manufacture of a medicament for the treatment or prophylaxis of occlusion of a vessel.

In one embodiment, the smooth muscle cells are vascular smooth muscle cells.

In one embodiment, the method further comprising administering a compound that inhibits proliferation of smooth muscle cells. In another embodiment, the compound that inhibits proliferation of smooth muscle cells is selected from paclitaxel, rapamycin, actinomycin D, or radioactivity.

In another embodiment, the vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

In another embodiment, the agent is selected from a nucleic acid, peptide, polypeptide, peptidomimetic, small organic molecule, antisense oligonucleotide, RNAi construct, ribozyme, or antibody.

In still another embodiment, the agent is formulated in a pharmaceutically acceptable carrier.

In an eleventh aspect, the invention provides a method for screening to identify and/or characterize agents that promote elastin signaling in vascular smooth muscle cells.

In a twelfth aspect, the invention provides a method for screening to identify and/or characterize an elastin receptor.

In a thirteenth aspect, the invention provides a method of conducting a drug discovery business.

The practice of the present invention will employ, unless otherwise indicated, conventional techniques of cell biology, cell culture, molecular biology, transgenic biology, microbiology, virology, recombinant DNA, and immunology, which are within the skill of the art. Such techniques are described in the literature. See, for example, Molecular Cloning: A Laboratory Manual, 3rd Ed., ed. by Sambrook and Russell (Cold Spring Harbor Laboratory Press: 2001); the treatise, Methods In Enzymology (Academic Press, Inc., N.Y.); Using Antibodies, Second Edition by Harlow and Lane, Cold Spring Harbor Press, New York, 1999; Current Protocols in Cell Biology, ed. by Bonifacino, Dasso, Lippincott-Schwartz, Harford, and Yamada, John Wiley and Sons, Inc., New York, 1999.

Other features and advantages of the invention will be apparent from the following detailed description, and from the claims.

### BRIEF DESCRIPTION OF THE DRAWINGS

FIG. 1: Aortagram of SVAS. A prominent stenosis just above the contracting heart in the ascending aorta is apparent.

FIG. 2: Cross sections of Eln +/+ (A-D) and Eln −/− aortae (E-H) at indicated developmental timepoints. Arterial structure stabilizes between embryonic day 15.5 (E15.5) and postnatal day 2.5 (P2.5) in Eln +/+ arteries. At E15.5, no difference was noted between Eln −/− and Eln +/+ arteries. However, subsequent timepoints revealed severe subendothelial accumulation of vsmcs in Eln −/− mice leading to luminal obliteration by P2.5.

FIG. 3: Elastin-vsmc interaction. In the normal mature state, elastin deposited and fixed in the extracellular matrix signals vascular smooth muscle cells to localize around the elastic lamella in organized rings and remain in a quiescent and contractile state. The disruption and destruction of elastic fibers by vascular injury or inflammation releases vsmcs to dedifferentiate, migrate, proliferate and occlude arteries. This model suggests that restoring elastin to an injured arterial wall would reduce obstructive vascular pathology.

FIG. 4: Elastin induces a mature contractile phenotype in vascular smooth muscle cells. (A-E) Immunofluorescence analysis using SM α-actin antisera reveals that (A) Eln +/+ vsmcs have a highly organized network of actin stress fibers, a hallmark of mature contractile vsmcs. In contrast, there is a paucity of actin stress fibers in Eln −/− vsmcs (B; outlined by white dots). Treatment with recombinant tropoelastin induces the formation of organized actin stress fibers in Eln −/− vsmcs (D), but does not affect Eln +/+ vascular smooth muscle cells (C). Scoring analysis (E). (F-J) Immunofluorescence analysis with vinculin antisera reveals that Eln +/+ vsmcs (F) have well-defined focal adhesions (arrows). In contrast, Eln −/− vsmcs (G; outlined by white dots) have poorly defined focal adhesions. Treatment with recombinant tropoelastin induces well-defined focal adhesions (arrows) throughout Eln −/− vsmcs (I), but does not affect vinculin organization in Eln +/+ vsmcs (H). Scoring analysis

FIG. 5: Effects of elastin on murine vsmc migration and proliferation. (A) Chemotaxis of different cell types to recombinant tropoelastin. (B) Migration of Eln +/+ and Eln −/− vsmcs in response to different concentration gradients of recombinant tropoelastin. The amount of elastin placed in the upper chamber where cells are originally seeded and the lower chambers are indicated. In (A) and (B), a modified Boyden-type chemotaxis chamber assay was used to determine the total number of migrated cells in 15 randomly selected high power microscope fields (HPF). (C) Elastin inhibits Eln −/− vsmc growth rates to level of Eln +/+ cells. Eln +/+ and Eln −/− vsmcs were passaged, seeded at the same density, and grown in Eln treated media (•) or untreated media (o). Cells were harvested 24, 48, and 72 hours after seeding and cell numbers were determined using a hemocytometer. Cell viability was assessed by trypan blue staining at the end of the experiment and determined to be >98%. Data shown are the mean±SD from 6 independent cell cultures.

FIG. 6: Elastin signaling is mediated through a G-protein coupled receptor and not an integrin receptor. (A) Induction of contractile organization in Eln −/− vsmcs by elastin is sensitive to pertussis toxin (Ptxn). However in the presence of the B-protomer of the toxin alone or EDTA there is little or no inhibition. (B) Migration of Eln −/− vsmcs towards elastin is also pertussis toxin sensitive but B-protomer and EDTA insensitive. The migration of Eln −/− vsmcs to platelet derived growth factor (PDGF: 30 ng/ml) is insensitive to pertussis toxin. The PDGF control indicates that pertussis toxin does not cause a general loss of migratory ability in Eln −/− vsmcs. EDTA insensitivity suggests that the elastin receptor is not in the integrin family, whereas pertussis toxin sensitivity suggests that the elastin receptor is a G-protein coupled receptor. In these preliminary experiments, the standard deviations for each value is within 20% of the means.

FIG. 7: Elastin activates Gi and Gs signaling pathways. (A) Cholera toxin increases the levels of cAMP in Eln −/− vsmcs. This elevation is caused by constitutive activation of the G s pathway. In the presence of elastin and cholera toxin, there is a marked decrease in cAMP indicating that elastin can reduce cAMP. These experiments indicate that elastin induces a receptor that activates Gi, inhibits adenylyl cyclase, and reduces cAMP levels. (B) Elastin treatment alone causes a statistically significant 3 fold increase in cAMP. Pertussis toxin is a specific inhibitor of Gi. Thus, in the presence of pertussis toxin, elastin's activation of Gi is blocked, and there is a marked accentuation of the levels of cAMP caused by Gs stimulation. Thus, these biochemical experiments indicate that elastin can activate both Gi and Gs.

FIG. 8: Reduction of restenosis by elastin sheath-stents in a porcine coronary injury model. (A) Sheaths of elastin matrix were isolated from porcine carotid arteries and examined by scanning electron microscopy. Cellular debris and collagen fibrils were not found with the elastin matrix sheath. Scale bar equals 10 μm. (B) Elastin matrix sheath covering an expanded metal stent or elastin sheath-stent. (C-D) Representative cross-sections taken from an artery treated with control stent alone (C) and an artery treated with the elastin sheath-stent (D). (E, F) Reduced neointimal accumulation over a range of mean injury scores in elastin sheath stents compared to control stents alone. Digital planimetry was used for quantitative assessment of percent luminal stenosis or neotintima thickness.

FIG. 9: Vascular smooth muscle cells derived from Elastin −/− mice are noncontractile. (A-B) Immunofluorescence using a polyclonal antibody that recognizes tropoelastin confirms that vascular smooth muscle cells derived from elastin +/+ mice express tropoelastin, whereas vascular smooth muscle cells derived from elastin −/− mice do not express tropoelastin. (C-D) H&E staining of vascular smooth muscle cells derived from elastin +/+ and −/− mice reveals substantial morphological differences. Cells derived from elastin +/+ mice have an elongated and spindle shaped-morphology which is consistent with a contractile phenotype. Cells derived from elastin −/− mice have a rounder, epitheliod morphology consistent with a non-contractile phenotype. (E-F) The presence of focal adhesions and actin stress fibers was assessed using an antibody against vinculin and rhodamine phalloidin, respectively. Cells derived from elastin +/+ mice are characterized by the presence of both actin stress fibers and focal adhesions, whereas cells derived from elastin −/− mice are characterized by a reduction or absence of both actin stress fibers and focal adhesions. (G-H) Transmission electron microscopy confirmed further differences in cellular architecture between elastin +/+ and −/− vascular smooth muscle cells. We note that both Golgi apparati (indicated with a large arrow) and rough endoplasmic reticula (indicated with a small arrow) are less abundant in elastin +/+ cells.

FIG. 10: Elastin peptides induce actin polymerization in elastin −/− vascular smooth muscle cells. (A-C) Immunofluorescence using rhodamine-conjugated phalloidin was used to assess the effect of elastin peptides on actin polymerization in elastin −/− cells. Treatment of the cells with an elastin peptide (SEQ ID NO: 3) induces actin myofilament polymerization (B), and the elastin peptide induces polymerization similarly to tropoelastin (SEQ ID NO: 2) (C). We note that treatment of the cells with control peptide (SEQ ID NO: 4) did not produce this effect and cells treated with control peptide exhibit only background staining of unpolymerized (globular) actin. (D-F) Immunofluorescence using an antibody which recognizes vinculin demonstrates that treatment of −/− cells with either an elastin peptide (SEQ ID NO: 3) (E) or tropoelastin (SEQ ID NO:2) (F) induces organization of focal adhesions, and this does not occur upon treatment with a control peptide (SEQ ID NO: 4) (D). (G-I) Merged images of actin, vinculin, and DAPI confirm that approximately equivalent numbers of cells are presented in each image.

FIG. 11: Elastin peptides induce actin polymerization through a dose dependent, temporally sensitive manner. (A-B) Summary graph scoring the percentage of −/− vascular smooth muscle cells with organized actin stress fibers and focal adhesions either in the absence of treatment, or following treatment with tropoelastin (SEQ ID NO: 2), an elastin peptide (SEQ ID NO: 3), or a control peptide (SEQ ID NO: 4). Scoring analysis confirms a significant increase in the percentage of cells with actin stress fibers and defined focal adhesions following treatment with either tropoelastin or with an elastin peptide. (C) Summary graph demonstrating that the increase of actin stress fibers in −/− vascular smooth muscle cells following treatment with an elastin peptide is dose dependent. (D) Summary graph demonstrating that stimulation of actin stress fiber formation by an elastin peptide is temporally sensitive. The effects occur after approximately one hour of treatment with an elastin peptide, and the maximal effect is observed after about 6 hours of treatment.

FIG. 12: Elastin peptides cause a threefold increase in the F:G actin ratio. (A) Western blot analysis of −/− vascular smooth muscle cells demonstrates that treatment with either tropoelastin (SEQ ID NO: 2) or with an elastin peptide (SEQ ID NO: 3) does not alter the protein levels of either α smooth muscle actin or vinculin. (B) The effect of tropoelastin and elastin peptides on vascular smooth muscle cells does not require translation. Pretreatment of −/− vascular smooth muscle cells with either actinomycin D or cyclohexamide does not block the effects of tropoelastin or elastin peptides. (C-D) A 3-fold increase in the F:G actin ratio was observed in cells treated with either tropoelastin (SEQ ID NO: 2) or elastin peptide (SEQ ID NO: 3), but not in untreated cells or cells treated with control peptide (SEQ ID NO: 4). Phalloidin, an F-actin stabilizing agent, served as a positive control and cytochalasin D, as F-actin destabilizing agent, served as a negative control.

FIG. 13: Elastin peptides mediate actin polymerization via the small GTPase, RhoA. (A) Following 3 hours of treatment with either tropoelastin (SEQ ID NO: 2) or elastin peptide (SEQ ID NO: 3), 70-80% of elastin −/− vascular smooth muscle cells exhibited actin stress fibers. This effect was significantly decreased if cells were pretreated with either C-3 coenzyme or Y-27632. C-3 coenzyme is a Rho GTPase inhibitor and Y-27632 is a Rho kinase inhibitor. (B) Treatment with elastin peptide or tropoelastin activates RhoA by facilitating the exchange of GDP for GTP. Briefly, cell lysates from treated or untreated −/− cells were immunoprecipitated using Rhotekin-coated beads which recognize GTP-bound RhoA. Western blot analysis of these lysates using an antibody against Rho demonstrates that both tropoelastin and elastin peptide result in a significant increase of GTP-bound (activated) RhoA. Rho-γGTP served as a positive control and Rho-GDP served as a negative control.

FIG. 14: Elastin peptides mediate actin polymerization via a G-protein-coupled signaling pathway. (A-B) Pretreatment of elastin −/− cells with the heterotrimeric G protein inhibitor pertussis toxin significantly decreases the effect of tropoelastin or elastin peptide on myofilament organization and on the F:G actin ratio, however pretreatment with the β protomer (inactivate form of pertussis toxin) does not have such an effect. (C-D) The effect of tropoelastin and elastin peptide on vascular smooth muscle cells are not altered by pretreatment with either EDTA or the receptor tyrosine kinase inhibitor genistein. (E) To examine the effect of elastin signaling on c AMP levels, −/− vascular smooth muscle cells were pretreated with forskolin to elevate basal cAMP levels. Subsequent treatment of these cells with tropoelastin or elastin peptide, but not with control peptide, resulted in a decrease of cAMP levels. (F) The effect of tropoelastin or elastin peptide on forskolin induced upregulation of cAMP levels is blocked by treatment with pertussis toxin but not by treatment with the α-protomer. (G) In contrast to the activation of RhoA observed when cells are treated with either tropoelastin or elastin peptide, cells pretreated with pertussis toxin are unable to activate RhoA.

FIG. 15: Elastin peptides mediate vascular smooth muscle cell chemotaxis through G-proteins. A modified Boyden-chamber assay was used to determine the total number of migrated cells in 15 randomly selected high-power microscopic fields (HPF). (A) 20 nM elastin peptide (SEQ ID NO: 3) regulated vascular smooth muscle migration in a manner comparable to both tropoelastin and PDGF. (B-C) The effect of elastin peptide was insensitive to treatment with either the integrin inhibitor EDTA or the tyrosine kinase inhibitor genistein. However, EDTA was able to block migration to the known integrin ligand collagen, and genistein was able to block migration to the receptor tyrosine kinase ligand PDGF. (D) Pertussis toxin, but not the inactive β-protomer, blocks the effects of elastin peptide on cell migration but has no impact on PDGF induced migration. (E) The Rho kinase inhibitor Y-27632 does not block the chemotactic activity of the elastin peptide.

FIG. 16: Elastin peptides activate GiRK channel activity. A2058 cells which are known to be responsive to elastin signaling were used in these studies. A2058 cells were transiently transfected with GiRK 1 and GiRK 4. Transfected cells were contacted with either tropoelastin, a peptide containing 7 repeats of the tropoelastin fragment VGVAPG, or a control peptide. Contacting cells with either the tropoelastin peptide or tropoelastin, but not a control peptide, resulted in activation of GiRK channel activity.

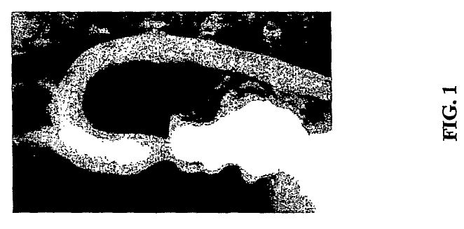

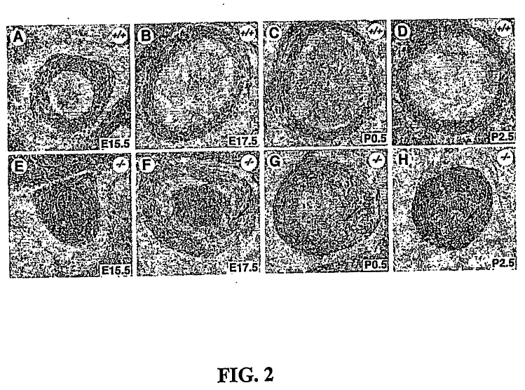

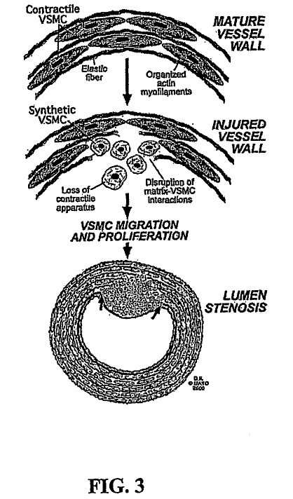

### DETAILED DESCRIPTION OF THE INVENTION

(i) Overview

Elastin signaling regulates the phenotypic change of vascular smooth muscle cells from a proliferative state to a quiescent state. In the proliferative state, which can occur in response to injury, the vascular smooth muscle cells can occlude a body vessel, such as an artery. Ironically, this proliferation may occur as a dedifferentiation from the quiescent state in response to treatment of an occlusion. This dedifferentiation is referred to as restenosis. This disease state frequently occurs following placement of intraluminal support devices, such as stents, at treatment sites in body vessels.

The presence of elastin at a site in a vessel prevents the vascular smooth muscle cells from dedifferentiating to a proliferative phenotype and thus occluding the vessel. Such occlusion is observed in a number of conditions collectively referred to as obstructive vascular diseases. These include diseases which result from lesions in a single gene such as SVAS, as well as diseases of complex etiology such as atherosclerosis. Furthermore, obstruction of vessels is often observed as a consequence of injury of the vessel such as can occur following angioplasty or the insertion of a catheter, wire, or other intraluminal device. Further examples of vessels in which obstruction can occur include, but are not limited to, common bile duct, pancreatic duct, esophagus, trachea, urethra, bladder, uterus, ovarian duct, Fallopian tube, vas deferens, prostatic duct, tear duct, and lymphatic duct.

Accordingly, the present invention provides methods of preventing occlusion of a body vessel by vascular smooth muscle cells by administering elastin or an agent that potentiates elastin signaling (herein referred to interchangeably as agents which potentiate elastin signaling, elastin activators, and agents that promote elastin signaling) thereby mimicking the effect of elastin on vascular smooth muscle cells. In a preferred embodiment, the method includes delivering one or more elastin activators to a treatment site in a body vessel. The body vessel can be any vessel that can be affected by occlusion by vascular smooth muscle cells including, but not limited to, arteries and veins. Further, the treatment site can be any site at which occlusion may occur, such as aneurysms, plaques, and points of weakening.

One or more agents can be delivered to the treatment site by any suitable method and/or apparatus. Preferably, because occlusion by vascular smooth muscle cells occurs frequently following placement of intraluminal support devices, the delivery of elastin is accomplished by delivery of an intraluminal support device that includes one or more agents that potentiate elastin signaling. An agent can of course be delivered in any other suitable form, such as other biomaterial forms, including films and sheets, as well as delivery as a soluble mediator. Specific methods of delivery can be selected by one of skill in the art in light of a variety of factors including, but not limited to, the particular agent to be administered, the particular vessel to be treated, and the overall health of the patient.

Furthermore, agents that promote elastin signaling include suitable fragments of tropoelastin that can be delivered to the treatment site or systemically. The exact fragments to be used must maintain the ability to drive the vascular smooth muscle cell phenotype towards the quiescent stage. An exemplary fragment of tropoelastin that can promote elastin signaling and influence the phenotype of vascular smooth muscle cells is VGVAPG (SEQ ID NO: 3).

Agents which potentiate elastin signaling may be delivered individually or in combination with one or more agents that potentiate elastin signaling. The invention further contemplates that agents which potentiate elastin signaling can be administered in combination with some other inhibitory therapy and/or agent. For example, one or more agents that potentiate elastin signaling may be delivered with an agent, such as paclitaxel and/or rapamycin. Compounds such as paclitaxel and rapamycin may help decrease proliferation in vascular smooth muscle cells by inducing a level of toxicity. Similarly, the invention contemplates that the subject agents may be administered as part of an overall treatment plan to address the particular obstructive vascular condition or the particular occlusion of a vessel in a given patient. For example, the subject agents may be administered in combination with one or more of: surgical intervention, angioplasty, cholesterol lowering medications, blood pressure lowering medications, blood thinners, dietary restrictions, stress reduction techniques, and the like.

The elastin receptor is a G-protein coupled receptor. Accordingly, any suitable method and/or agent that can be used to activate the signaling pathway associated with the elastin receptor c an be utilized in the methods of the present invention. We summarize herein data which has helped improve our understanding of specific components of the elastin signaling pathway, and the invention contemplates that agents which potentiate elastin signaling by modulating any portion of this pathway will mimick the effects of elastin on vascular smooth muscle cells. Accordingly, one or more agents which potentiates elastin signaling may be useful in the subject methods.

The Elastin Pathway

(a) G-Protein Coupled Receptor: The elastin receptor is a G-protein coupled receptor. Ligand binding to the elastin receptor (e.g., binding of tropoelastin or binding of a bioactive fragment of tropoelastin) results in a conformational change in the elastin receptor and subsequent activation of downstream components of the elastin signaling pathway.

Given the role of elastin signaling in vascular smooth muscle cell proliferation, differentiation, as well as other cellular and morphological properties, and given the role of elastin receptor, a G-protein coupled receptor, in transducing the elastin signal, agents which activate the elastin receptor can be used in the subject methods to influence vascular smooth muscle cells. Agents for use in the subject methods include agents that bind to and activate the elastin receptor, as well as agents which potentiate the activity and/or expression of the elastin receptor independent of receptor binding. Further exemplary agents include constituitively active variants of the elastin receptor capable of potentiating elastin signaling in the absence of ligand binding.

The invention further contemplates methods of screening for agents which activate the elastin receptor. Such agents, including nucleic acids, peptides, polypeptides, peptidometics, antisense constructs or oligonucleotides, RNAi constructs or oligonucleotides, antibodies, small organic molecules, and the like, can be identified based on their ability to influence one or more properties of vascular smooth muscle cells. Agents identified by the screening methods disclosed herein may be used to influence one or more properties of vascular smooth muscle cells.

(b) Heterotrimeric G proteins: Elastin signaling is transduced via Gi heterotrimeric G proteins. Briefly, heterotrimeric G proteins interact with an intracellular domain of an elastin receptor to transduce signaling intracellularly. Activation of the elastin receptor results in activation of Gi heterotrimeric G proteins.

Given the role of elastin signaling in vascular smooth muscle cell proliferation, differentiation, as well as other cellular and morphological properties, and given the role of heterotrimeric G proteins (specifically Gi) in transducing the elastin signal, agents which activate heterotrimeric Gi proteins can be used in the subject methods to influence vascular smooth muscle cells. Agents for use in the subject methods include agents that bind to and activate Gi proteins, as well as agents which activate Gi proteins indirectly by binding to and activating other proteins which increase the expression and/or activity of Gi proteins.

The invention further contemplates methods of screening for agents which activate heterotrimeric Gi proteins. Such agents, including nucleic acids, peptides, polypeptides, peptidometics, antisense constructs or oligonucleotides, RNAi constructs or oligonucleotides, antibodies, small organic molecules, and the like, can be identified based on their ability to influence one or more properties of vascular smooth muscle cells. Agents identified by the screening methods disclosed herein may be used to influence one or more properties of vascular smooth muscle cells.

(c) RhoA: The methods and compositions disclosed in this application are based, in part, on altering the activity of Rho family GTPases in vascular smooth muscle cells in order to influence the differentiation and/or other cellular and morphological properties of vascular smooth muscle cells. Briefly, Rho family GTPases represent a point of convergence for extracellular signals that modulate the actin cytoskeleton. Rho family GTPases, including Rho, Rac and Cdc42, regulate the actin cytoskeleton (reviewed in Hall and Nobes (2000) Philos Trans R Soc Lond B Biol Sci. 355: 965-70; Luo et al. (2000) Nature Rev. Neurosci. 1: 173-180). As used herein, the term “RhoA” and “RhoA GTPase” will be used interchangeably. Wildtype Rho family GTPases cycle between two biochemical states characterized by association with either GDP or GTP. The GTP bound state is the “on” state and the GDP bound state is the “off” state, and Rho family GTPases will exert varying effects on cells in their “on” or “off” conformation. The “on” or “off” state is regulated, at least in part, by several other classes of proteins including guanine nucleotide exchange factors (GEFs), GTPase activating proteins (GAPs), and GTP dissociation inhibitors (GDIs). GEFs act to facilitate the release of GDP and the subsequent binding of GTP, and thus promote the “on” state of Rho family GTPases. GAPs function to stimulate the intrinsic GTP hydrolyzing activity of Rho family GTPases, and thus promote the “off” state. GDIs often act as negative regulators by preventing the dissociation of GDP from Rho family GTPases, and thus promote the “off” state.

A large number of GEFs, GAPs, and GDIs exist. Some of these are expressed specifically in certain tissues or cell types. For example, the GEFs Kalirin, Ost, Stef and Tiam, the GAPs p190RhoGAP, α-chimaerin, and oligophrenin-1, and the GDI RhoGDIγ, are all enriched in the nervous system. Additionally, it is known that some GEFs, GAPs, and GDIs preferentially regulate a particular Rho family GTPase, while other GEFs, GAPs, and GDIs can regulate more than one Rho family GTPase, and still other GEFs, GAPs, and GDIs can regulate many Rho family GTPases (Nikolic (2002) International Journal of Biochemistry and Cell Biology 34: 731-745; Settleman (2001) Developmental Cell 1: 321-331).

Given the role of elastin signaling in vascular smooth muscle cell proliferation, differentiation, as well as other cellular and morphological properties, and given the role of Rho family GTPases (specifically RhoA) in transducing the elastin signal, agents which activate Rho family GTPases can be used in the subject methods to influence vascular smooth muscle cells. Agents for use in the subject methods include agents that promote the “on” state of a class of Rho family GTPases. In one embodiment, an agent specifically promotes the “on” state of RhoA GTPases, and does not promote the “on” state of non-RhoA family Rho GTPases. In another embodiment, an agent promotes the “on” state of RhoA GTPases, but also promotes the “on” state of one or more other Rho family GTPase.

The invention further contemplates methods of screening for agents which promote the “on” state of Rho family GTPases. Such agents, including nucleic acids, peptides, polypeptides, peptidometics, antisense constructs or oligonucleotides, RNAi constructs or oligonucleotides, antibodies, small organic molecules, and the like, can be identified based on their ability to influence one or more properties of vascular smooth muscle cells. Agents identified by the screening methods disclosed herein may be used to influence one or more properties of vascular smooth muscle cells. The invention contemplates that agents identified by the subject screening methods may specifically activate only RhoA GTPases, and such agents would not activate other Rho family GTPases. The invention further contemplates that agents identified by the subject screening methods may activate RhoA GTPases, as well as members of one or more family of Rho GTPases. Screening methodologies can be modified in order to preferentially identify either agents which activate only RhoA GTPases, or to identify agents which activate both RhoA GTPases and members of one or more other family of GTPases.

(ii) Definitions

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

As used herein, “Rho family GTPase” refers to a broad class of proteins that have intrinsic GTPase activity (reviewed in Wittinghofer and Nassar (1996) TIBS 21: 488-491; Ridley (2001) Journal of Cell Science 114: 2713-2722; Nikolic et al. (2002) International Journal of Biochemistry and Cell Biology 34: 731-745). Intrinsic GTPase activity means that members of this family bind to GTP and convert GTP to GDP. These proteins function as molecular switches. For the native enzymes, in the GTP bound form they are in an active conformation or “on” state and are able to interact with a set of downstream effectors. In their GDP bound state they are in an “off” conformation and may bind to a different set of proteins.

Mutants can be generated to make the Rho family GTPase constituitively active or function in a dominant negative capacity. These mutants may be preferentially GTP or GDP bound, respectively. However in some cases, the mutants can assume the “on” or “off” state independent of nucleotide binding.

Exemplary Rho family GTPases include Cdc42, Rac1, and RhoA. A number of other classes of molecules interact with Rho family GTPases to promote either the GTP or GDP bound states including guanine nucleotide exchange factors (GEFs), GTPase activating proteins (GAPs), and GTP dissociation inhibitors (GDIs). GEFs act to facilitate the release of GDP and the subsequent rebinding of GTP, and act to promote the “on” state (activate) of Rho family GTPases. GAPs function to stimulate the intrinsic GTP hydrolyzing activity of Rho family GTPases, and thus act to promote the “off” state. GDIs often act as negative regulators by preventing the dissociation of GDP from Rho family GTPases, and thus promote the “off” state.

An agent which promotes or increases the active conformation or the “on” state of a Rho family GTPase will be referred to as an “activator”. Such agents may include constitutively active mutant forms of the Rho family GTPases, wherein the mutant mimics the active or “on state.” An agent which promotes the “off” state, whether by actively promoting the “off” state or by inhibiting the “on” state, will be referred to as an “inhibitor”. Such agents may include dominant negative mutant forms of the Rho family GTPases, wherein the mutant promotes the “off state”.

The term “agent” refers to a compound used in the methods of the present invention to influence one or more properties of vascular smooth muscle cells, as well as to a compound screened by the methods of the present invention. The term agent includes nucleic acids, peptides, proteins, peptidomimetics, small organic molecules, chemical compounds, ribozymes, RNAi constructs (including siRNA), antisense RNAs, and antibodies. Preferred agents for use in the subject methods are those which influence one or more property of vascular smooth muscle cells. Further preferred agents are those which decrease or prevent occlusion of a vessel, and agents which decrease or prevent vascular occlusion.

Agents which promote elastin signaling can be administered and/or screened individually, or can be administered in combination with one or more other agents which promote elastin signaling. When combinations of agents are used, these combinations may function at the same point in the elastin signaling pathway (for example, at the level of activation of RhoA) or these agents can act at different point in the elastin signaling pathway. Additionally, the invention contemplates that one or more agents that promote elastin signaling can be used in combination with other therapies used to prevent or treat the particular injury, disease, or condition.

The invention further contemplates the screening of libraries of agents. Such libraries may include, without limitation, cDNA libraries (either plasmid based or phage based), expression libraries, combinatorial libraries, chemical libraries, phage display libraries, variegated libraries, and biased libraries. The term “library” refers to a collection of nucleic acids, proteins, peptides, chemical compounds, small organic molecules, or antibodies. Libraries comprising each of these are well known in the art. Exemplary types of libraries include combinatorial, variegated, biased, and unbiased libraries. Libraries can provide a systematic way to screen large numbers of nucleic acids, proteins, peptides, chemical compounds, small organic molecules, or antibodies. Often, libraries are sub-divided into pools containing some fraction of the total species represented in the entire library. These pools can then be screened to identify fractions containing the desired activity. The pools can be further subdivided, and this process can be repeated until either (i) the desired activity can be correlated with a specific species contained within the library, or (ii) the desired activity is lost during further subdivision of the pool of species, and thus is the result of multiple species contained within the library.

As used herein, “protein” is a polymer consisting essentially of any of the 20 amino acids. Although “polypeptide” is often used in reference to relatively large polypeptides, and “peptide” is often used in reference to small polypeptides, usage of these terms in the art overlaps and is varied.

The terms “peptide(s)”, “protein(s)” and “polypeptide(s)” are used interchangeably herein.

The terms “polynucleotide sequence” and “nucleotide sequence” are also used interchangeably herein.

“Recombinant,” as used herein, means that a protein is derived from a prokaryotic or eukaryotic expression system.

The term “wild type” refers to the naturally-occurring polynucleotide sequence encoding a protein, or a portion thereof, or protein sequence, or portion thereof, respectively, as it normally exists in vivo.

The term “mutant” refers to any change in the genetic material of an organism, in particular a change (i.e., deletion, substitution, addition, or alteration) in a wild type polynucleotide sequence or any change in a wild type protein. The term “variant” is used interchangeably with “mutant”. Although it is often assumed that a change in the genetic material results in a change of the function of the protein, the terms “mutant” and “variant” refer to a change in the sequence of a wildtype protein regardless of whether that change alters the function of the protein (e.g., increases, decreases, imparts a new function), or whether that change has no effect on the function of the protein (e.g., the mutation or variation is silent).

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

As used herein, the term “gene” or “recombinant gene” refers to a nucleic acid comprising an open reading frame encoding a polypeptide, including both exon and (optionally) intron sequences.

As used herein, the term “vector” refers to a nucleic acid molecule capable of transporting another nucleic acid to which it has been linked. Preferred vectors are those capable of autonomous replication and/or expression of nucleic acids to which they are linked. Vectors capable of directing the expression of genes to which they are operatively linked are referred to herein as “expression vectors”.

A polynucleotide sequence (DNA, RNA) is “operatively linked” to an expression control sequence when the expression control sequence controls and regulates the transcription and translation of that polynucleotide sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence.

“Transcriptional regulatory sequence” is a generic term used throughout the specification to refer to nucleic acid sequences, such as initiation signals, enhancers, and promoters, which induce or control transcription of protein coding sequences with which they are operably linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein.

As used herein, the term “tissue-specific promoter” means a nucleic acid sequence that serves as a promoter, i.e., regulates expression of a selected nucleic acid sequence operably linked to the promoter, and which affects expression of the selected nucleic acid sequence in specific cells of a tissue, such as cells of neural origin, e.g. neuronal cells. The term also covers so-called “leaky” promoters, which regulate expression of a selected nucleic acid primarily in one tissue, but cause expression in other tissues as well.

“Identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules are identical at that position. A degree of identity between sequences is a function of the number of matching positions shared by the sequences.

A “chimeric protein” or “fusion protein” is a fusion of a first amino acid sequence encoding a polypeptide with a second amino acid sequence defining a domain (e.g. polypeptide portion) foreign to and not substantially homologous with any domain of the first polypeptide. A chimeric protein may present a foreign domain which is found (albeit in a different protein) in an organism which also expresses the first protein, or it may be an “interspecies”, “intergenic”, etc. fusion of protein structures expressed by different kinds of organisms.

The “non-human animals” of the invention include mammals such as rats, mice, rabbits, sheep, cats, dogs, cows, pigs, and non-human primates.

The term “isolated” as used herein with respect to nucleic acids, such as DNA or RNA, refers to molecules separated from other DNAs, or RNAs, respectively, that are present in the natural source of the macromolecule. For example, an isolated nucleic acid encoding a Rho family GTPase preferably includes no more than 10 kilobases (kb) of nucleic acid sequence which naturally immediately flanks the Rho family GTPase gene in genomic DNA, more preferably no more than 5 kb of such naturally occurring flanking sequences, and most preferably less than 1.5 kb of such naturally occurring flanking sequence. The term isolated as used herein also refers to a nucleic acid or peptide that is substantially free of cellular material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state.

As used herein, “proliferating” and “proliferation” refer to cells undergoing mitosis.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the animal's system and, thus, is subject to metabolism and other like processes, for example, subcutaneous administration.

The phrase “effective amount” as used herein means that the amount of one or more agent, material, or composition comprising one or more agents as described herein which is effective for producing some desired effect in a subject; for example, an amount of the compositions described herein effective to decrease or prevent occlusion of a vessel.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

Functional equivalents of a polypeptide, a polypeptide fragment, or a variant polypeptide are those polypeptides that retain a biological and/or an immunological activity of the native or naturally-occurring polypeptide. Immunological activity refers to the ability to induce the production of an antibody against an antigenic epitope possessed by a native polypeptide; biological activity refers to a function, either inhibitory or stimulatory, caused by the particular native polypeptide that excludes immunological activity. In the context of the present invention, exemplary biological activities include the ability to promote elastin signaling in vascular smooth muscle cells. Further exemplary biological activities include the ability to bind to a particular receptor, the ability to activate transcription of a particular gene, the ability to inhibit transcription of a particular gene, the ability to associate (e.g., directly or indirectly associate) with a particular cofactor, the ability to promote signaling via a particular signal transduction pathway, and the ability to inhibit signaling via another particular signal transduction pathway. “Derivatives” of nucleic acid sequences or amino acid sequences are formed from the native compounds either directly or by modification or partial substitution. “Analogs” are nucleic acid sequences or amino acid sequences that have a structure similar to, but not identical to, the native compound but differ from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are identical to the nucleic acids or proteins of the invention. In various embodiments, the derivatives or analogs are at least about 70%, 75%, 80%, 85%, 90%, 95%, 97%, 98%, 99%, or greater than 99% identical to a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions.

A “homologous nucleic acid sequence” or “homologous amino acid sequence,” or variations thereof, refer to sequences characterized by a degree of identity at the nucleotide level or amino acid level as discussed above. Homologous nucleotide sequences encode those sequences coding for isoforms of a particular sequence. Isoforms can be expressed in different tissues of the same organism as a result of, for example, alternative splicing of RNA. Alternatively, different genes can encode isoforms. Homologous nucleotide sequences include nucleotide sequences encoding a polypeptide from other species, including, but not limited to: vertebrates, and thus can include, e.g., human, frog, mouse, rat, rabbit, dog, cat, cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding a particular protein. Homologous nucleic acid sequences include those nucleic acid sequences that encode conservative amino acid substitutions (see below).

“Small molecule” as used herein, is meant to refer to a composition, which has a molecular weight of less than about 5 kD and most preferably less than about 4 kD. Small molecules can be nucleic acids, peptides, polypeptides, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened to identify compounds (e.g., small organic molecules) that have a desired activity.

As used herein, “G-protein coupled receptor” refers to the a class of seven-transmembrane spanning receptors w ell known in the art. A large number of G-protein coupled receptors have been identified, and the ligands for these receptors include nucleic acids, peptides, proteins, hormones, small organic molecules, and the like. Ligand binding to an extracellular binding site of a G-protein coupled receptor results in a conformational change in the protein which allows the activation of a heterotrimeric G protein intracellularly. Intracellular activation of a heterotrimeric G protein results in further downstream events which ultimately result in the transduction of an extracellular signal intracellularly. The mechanistic specifics and cellular responses to activation of a particular G-protein coupled receptor will vary depending on the ligand and G-protein coupled receptor.

As used herein, “tropoelastin” refers to the protein encoded by the elastin gene. The nucleic acid sequence of human tropoelastin is represented in SEQ ID NO: 1 and the amino acid sequence is represented in SEQ ID NO: 2. Tropoelastin is able to stimulate elastin signaling, and stimulation of elastin signaling has one or more of the following functional consequences including, but not limited to: (i) increase actin stress fiber formation in vascular smooth muscle cells, (ii) increase expression of vinculin in vascular smooth muscle cells, (iii) increase focal adhesion formation in vascular smooth muscle cells, (iv) inhibit dedifferentiation of vascular smooth muscle cells, (v) promote actin polymerization in vascular smooth muscle cells, (vi) increase the ratio of F:G actin in vascular smooth muscle cells, (vii) decrease or inhibit occlusion of a vessel, (viii) decrease or inhibit vascular obstruction, (ix) decrease or inhibit restenosis, and/or (x) prevent stenosis.

In addition to full length tropoelastin, bioactive fragments of tropoelastin can stimulate elastin signaling. By bioactive fragment is meant that a given portion of the protein maintains one or more of the functional attributes of the full length protein. In the context of the present invention, a bioactive fragment of tropoelastin maintains the ability to promote elastin signaling and thus results in one or more of the functional consequences of elastin signaling including, but not limited to, (i) increase actin stress fiber formation in vascular smooth muscle cells, (ii) increase expression of vinculin in vascular smooth muscle cells, (iii) increase focal adhesion formation in vascular smooth muscle cells, (iv) inhibit dedifferentiation of vascular smooth muscle cells, (v) promote actin polymerization in vascular smooth muscle cells, (vi) increase the ratio of F:G actin in vascular smooth muscle cells, (vii) decrease or inhibit occlusion of a vessel, (viii) decrease or inhibit vascular obstruction, (ix) decrease or inhibit restenosis, and/or (x) prevent stenosis. An exemplary bioactive fragment of tropoelastin is provided in SEQ ID NO: 3. The invention contemplates the use not only of bioactive peptide fragments of tropoelastin, but also peptidomimetics (modified fragments). An exemplary peptidomimetic of tropoelastin is a peptidomimetic of SEQ ID NO: 3.

(iii) Exemplary Agents

The present invention contemplates that numerous agents can be used to promote elastin signaling in vascular smooth muscle cells, either in vivo or in vitro, and such agents are useful for modulating the proliferation, differentiation, and cellular and morphological behavior of vascular smooth muscle cells, either in vivo or in vitro. Agents which promote elastin signaling, either in vivo or in vitro, are useful in the methods of the present invention. Without being bound by theory, such agents include nucleic acids, peptides, polypeptides, peptidomimetics, small organic molecules, antibodies, antisense oligonucleotides, RNAi constructs, ribozymes, and the like. Furthermore, it is appreciated that an agent which promotes elastin signaling, whether via a known or an unknown mechanism, is useful in the methods of the present invention. Nevertheless, and without being bound by theory, the invention contemplates that exemplary agents include: (i) agents that promote the binding of elastin or a bioactive fragment to its receptor, (ii) agents that increase the expression and/or activity of tropoelastin, (iii) agents that activate the elastin G-protein coupled receptor in the presence or absence of ligand, (iv) agents that increase the expression and/or activity of the heterotrimeric Gi protein activated in response to elastin signaling, (v) agents that increase the expression and/or activity of RhoA GTPase which is activated in response to elastin signaling, (vi) agents that inhibit the expression and/or activity of an agent that normally functions to inhibit the activity of Gi, and (vii) agents that inhibit the expression and/or activity of an agent that normally functions to inhibit the activity of RhoA. Examples of each of these classes of agents will be described in further detail herein.

(a) Classes of Agents

Numerous mechanisms exist to promote or inhibit the expression and/or activity of a particular mRNA or protein. The present invention provides methods and compositions for promoting elastin signaling. Without being bound by theory, the present invention contemplates that elastin signaling can be potentiated either by increasing the activity of a protein which positively regulates elastin signal transduction, or by inhibiting the activity of a protein which negatively regulates elastin signal transduction.

The invention contemplates the use of any of a number of methods for promoting the expression and/or activity of a particular mRNA or protein, as well as a number of methods for inhibiting the expression and/or activity of a particular mRNA or protein. Additionally, the invention contemplates combinatorial methods comprising either (i) the use of two or more agents that decrease the expression and/or activity of a particular mRNA or protein, (ii) the use of one or more agents that decrease the expression and/or activity of a particular mRNA or protein plus the use of one or more agents that decrease the expression and/or activity of a second mRNA or protein, (iii) the use of two or more agents that increase the expression and/or activity of a particular mRNA or protein, (iv) the use of one or more agents that increase the expression and/or activity of a particular mRNA or protein plus the use of one or more agents that increase the expression and/or activity of a second mRNA or protein, (v) the use of one or more agents that increase expression and/or activity of a particular mRNA or protein plus the use of one or more agents that decrease the expression and/or activity of a particular mRNA or protein.

The following are illustrative examples of methods for promoting or inhibiting the expression and/or activity of an mRNA or protein. These examples are in no way meant to be limiting, and one of skill in the art can readily select from among known methods of promoting or inhibiting expression and/or activity of genes and proteins.

Antisense oligonucleotides are relatively short nucleic acids that are complementary (or antisense) to the coding strand (sense strand) of the mRNA encoding a particular protein. Although antisense oligonucleotides are typically RNA based, they can also be DNA based. Additionally, antisense oligonucleotides are often modified to increase their stability.

Without being bound by theory, the binding of these relatively short oligonucleotides to the mRNA is believed to induce stretches of double stranded RNA that trigger degradation of the messages by endogenous RNAses. Additionally, sometimes the oligonucleotides are specifically designed to bind near the promoter of the message, and under these circumstances, the antisense oligonucleotides may additionally interfere with translation of the message. Regardless of the specific mechanism by which antisense oligonucleotides function, their administration to a cell or tissue allows the degradation of the mRNA encoding a specific protein. Accordingly, antisense oligonucleotides decrease the expression and/or activity of a particular protein.

The oligonucleotides can be DNA or RNA or chimeric mixtures or derivatives or modified versions thereof, single-stranded or double-stranded. The oligonucleotide can be modified at the base moiety, sugar moiety, or phosphate backbone, for example, to improve stability of the molecule, hybridization, etc. The oligonucleotide may include other appended groups such as peptides (e.g., for targeting host cell receptors), or agents facilitating transport across the cell membrane (see, e.g., Letsinger et al., 1989, Proc. Natl. Acad. Sci. U.S.A. 86:6553-6556; Lemaitre et al., 1987, Proc. Natl. Acad. Sci. 84:648-652; PCT Publication No. WO88/09810, published Dec. 15, 1988) or the blood-brain barrier (see, e.g., PCT Publication No. WO89/10134, published Apr. 25, 1988), hybridization-triggered cleavage agents (See, e.g., Krol et al., 1988, BioTechniques 6:958-976) or intercalating agents. (See, e.g., Zon, 1988, Pharm. Res. 5:539-549). To this end, the oligonucleotide may be conjugated to another molecule.

The antisense oligonucleotide may comprise at least one modified base moiety which is selected from the group including but not limited to 5-fluorouracil, 5-bromouracil, 5-chlorouracil, 5-iodouracil, hypoxanthine, xanthine, 4-acetylcytosine, 5-(carboxyhydroxytriethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methyl ester, uracil-5-oxyacetic acid (v), 5-methyl-2-thiouracil, 3-(3-amino-3-N-2-carboxypropyl) uracil, (acp3)w, and 2,6-diaminopurine.

The antisense oligonucleotide may also comprise at least one modified sugar moiety selected from the group including but not limited to arabinose, 2-fluoroarabinose, xylulose, and hexose.

The antisense oligonucleotide can also contain a neutral peptide-like backbone. Such molecules are termed peptide nucleic acid (PNA)-oligomers and are described, e.g., in Perry-O'Keefe et al. (1996) Proc. Natl. Acad. Sci. U.S.A. 93:14670 and in Eglom et al. (1993) Nature 365:566. One advantage of PNA oligomers is their capability to bind to complementary DNA essentially independently from the ionic strength of the medium due to the neutral backbone of the DNA. In yet another embodiment, the antisense oligonucleotide comprises at least one modified phosphate backbone selected from the group consisting of a phosphorothioate, a phosphorodithioate, a phosphoramidothioate, a phosphoramidate, a phosphordiamidate, a methylphosphonate, an alkyl phosphotriester, and a formacetal or analog thereof.

In yet a further embodiment, the antisense oligonucleotide is an -anomeric oligonucleotide. An -anomeric oligonucleotide forms specific double-stranded hybrids with complementary RNA in which, contrary to the usual units, the strands run parallel to each other (Gautier et al., 1987, Nucl. Acids Res. 15:6625-6641). The oligonucleotide is a 2′-O-methylribonucleotide (Inoue et al., 1987, Nucl. Acids Res. 15:6131-6148), or a chimeric RNA-DNA analogue (Inoue et al., 1987, FEBS Lett. 215:327-330).

Oligonucleotides of the invention may be synthesized by standard methods known in the art, e.g., by use of an automated DNA synthesizer (such as are commercially available from Biosearch, Applied Biosystems, etc.). As examples, phosphorothioate oligonucleotides may be synthesized by the method of Stein et al. (1988, Nucl. Acids Res. 16:3209), methylphosphonate oligonucleotides can be prepared by use of controlled pore glass polymer supports (Sarin et al., 1988, Proc. Natl. Acad. Sci. U.S.A. 85:7448-7451), etc.

The selection of an appropriate oligonucleotide can be readily performed by one of skill in the art. Given the nucleic acid sequence encoding a particular protein, one of skill in the art can design antisense oligonucleotides that bind to that nucleic acid sequence, and test these oligonucleotides in an in vitro or in vivo system to confirm that they bind to and mediate the degradation of the mRNA encoding the particular protein. To design an antisense oligonucleotide that specifically binds to and mediates the degradation of a message encoding a particular protein, it is important that the sequence recognized by the oligonucleotide is unique or substantially unique to that particular protein. For example, sequences that are frequently repeated across protein may not be an ideal choice for the design of an oligonucleotide that specifically recognizes and degrades a particular message. One of skill in the art can design an oligonucleotide, and compare the sequence of that oligonucleotide to nucleic acid sequences that are deposited in publicly available databases to confirm that the sequence is specific or substantially specific for a particular protein.

In another example, it may be desirable to design an antisense oligonucleotide that binds to and mediates the degradation of more than one message. In one example, the messages may encode related proteins such as isoforms or functionally redundant proteins. In such a case, one of skill in the art can align the nucleic acid sequences that encode these related proteins, and design an oligonucleotide that recognizes both messages.

A number of methods have been developed for delivering antisense DNA or RNA to cells; e.g., antisense molecules can be injected directly into the tissue site, or modified antisense molecules, designed to target the desired cells (e.g., antisense linked to peptides or antibodies that specifically bind receptors or antigens expressed on the target cell surface) can be administered systematically.

However, it may be difficult to achieve intracellular concentrations of the antisense sufficient to suppress translation on endogenous mRNAs in certain instances. Therefore another approach utilizes a recombinant DNA construct in which the antisense oligonucleotide is placed under the control of a strong pol III or pol II promoter. For example, a vector can be introduced in vivo such that it is taken up by a cell and directs the transcription of an antisense RNA. Such a vector can remain episomal or become chromosomally integrated, as long as it can be transcribed to produce the desired antisense RNA. Such vectors can be constructed by recombinant DNA technology methods standard in the art. Vectors can be plasmid, viral, or others known in the art, used for replication and expression in mammalian cells. Expression of the sequence encoding the antisense RNA can be by any promoter known in the art to act in mammalian, preferably human cells. Such promoters can be inducible or constitutive. Such promoters include but are not limited to: the SV40 early promoter region (Bernoist and Chambon, 1981, Nature 290:304-310), the promoter contained in the 3′ long terminal repeat of Rous sarcoma virus (Yamamoto et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al, 1982, Nature 296:39-42), etc. Any type of plasmid, cosmid, YAC or viral vector can be used to prepare the recombinant DNA construct that can be introduced directly into the tissue site. Alternatively, viral vectors can be used which selectively infect the desired tissue, in which case administration may be accomplished by another route (e.g., systematically).

RNAi constructs comprise double stranded RNA that can specifically block expression of a target gene. “RNA interference” or “RNAi” is a term initially applied to a phenomenon observed in plants and worms where double-stranded RNA (dsRNA) blocks gene expression in a specific and post-transcriptional manner. Without being bound by theory, RNAi appears to involve mRNA degradation, however the biochemical mechanisms are currently an active area of research. Despite some mystery regarding the mechanism of action, RNAi provides a useful method of inhibiting gene expression in vitro or in vivo.

As used herein, the term “dsRNA” refers to siRNA molecules, or other RNA molecules including a double stranded feature and able to be processed to siRNA in cells, such as hairpin RNA moieties.

The term “loss-of-function,” as it refers to genes inhibited by the subject RNAi method, refers a diminishment in the level of expression of a gene when compared to the level in the absence of RNAi constructs.

As used herein, the phrase “mediates RNAi” refers to (indicates) the ability to distinguish which RNAs are to be degraded by the RNAi process, e.g., degradation occurs in a sequence-specific manner rather than by a sequence-independent dsRNA response, e.g., a PKR response.

As used herein, the term “RNAi construct” is a generic term used throughout the specification to include small interfering RNAs (siRNAs), hairpin RNAS, and other RNA species which can be cleaved in vivo to form siRNAs. RNAi constructs herein also include expression vectors (also referred to as RNAi expression vectors) capable of giving rise to transcripts which form dsRNAs or hairpin RNAs in cells, and/or transcripts which can produce siRNAs in vivo.

“RNAi expression vector” (also referred to herein as a “dsRNA-encoding plasmid”) refers to a replicable nucleic acid constructs used to express (transcribe) RNA which produces siRNA moieties in the cell in which the construct is expressed. Such vectors include a transcriptional unit comprising an assembly o f (1) genetic element(s) having a regulatory role in gene expression, for example, promoters, operators, or enhancers, operatively linked to (2) a “coding” sequence which is transcribed to produce a double-stranded RNA (two RNA moieties that anneal in the cell to form an siRNA, or a single hairpin RNA which can be processed to an siRNA), and (3) appropriate transcription initiation and termination sequences. The choice of promoter and other regulatory elements generally varies according to the intended host cell. In general, expression vectors of utility in recombinant DNA techniques are often in the form of “plasmids” which refer to circular double stranded DNA loops which, in their vector form are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

The RNAi constructs contain a nucleotide sequence that hybridizes under physiologic conditions of the cell to the nucleotide sequence of at least a portion of the mRNA transcript for the gene to be inhibited (i.e., the “target” gene). The double-stranded RNA need only be sufficiently similar to natural RNA that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and the RNAi construct sequence is no more than 1 in 5 basepairs, or 1 in 10 basepairs, or 1 in 20 basepairs, or 1 in 50 basepairs. Mismatches in the center of the siRNA duplex are most critical and may essentially abolish cleavage of the target RNA. In contrast, nucleotides at the 3′ end of the siRNA strand that is complementary to the target RNA do not significantly contribute to specificity of the target recognition.

Sequence identity may be optimized by sequence comparison and alignment algorithms known in the art (see Gribskov and Devereux, Sequence Analysis Primer, Stockton Press, 1991, and references cited therein) and calculating the percent difference between the nucleotide sequences by, for example, the Smith-Waterman algorithm as implemented in the BESTFIT software program using default parameters (e.g., University of Wisconsin Genetic Computing Group). Greater than 90% sequence identity, or even 100% sequence identity, between the inhibitory RNA and the portion of the target gene is preferred. Alternatively, the duplex region of the RNA may be defined functionally as a nucleotide sequence that is capable of hybridizing with a portion of the target gene transcript (e.g., 400 mM NaCl, 40 mM PIPES pH 6.4, 1 mM EDTA, 50° C. or 70° C. hybridization for 12-16 hours; followed by washing).

Production of RNAi constructs can be carried out by chemical synthetic methods or by recombinant nucleic acid techniques. Endogenous RNA polymerase of the treated cell may mediate transcription in vivo, or cloned RNA polymerase can be used for transcription in vitro. The RNAi constructs may include modifications to either the phosphate-sugar backbone or the nucleoside, e.g., to reduce susceptibility to cellular nucleases, improve bioavailability, improve formulation characteristics, and/or change other pharmacokinetic properties. For example, the phosphodiester linkages of natural RNA may be modified to include at least one of an nitrogen or sulfur heteroatom. Modifications in RNA structure may be tailored to allow specific genetic inhibition while avoiding a general response to dsRNA. Likewise, bases may be modified to block the activity of adenosine deaminase. The RNAi construct may be produced enzymatically or by partial/total organic synthesis, any modified ribonucleotide can be introduced by in vitro enzymatic or organic synthesis.

Methods of chemically modifying RNA molecules can be adapted for modifying RNAi constructs (see, for example, Heidenreich et al. (1997) Nucleic Acids Res, 25:776-780; Wilson et al. (1994) J Mol Recog 7:89-98; Chen et al. (1995) Nucleic Acids Res 23:2661-2668; Hirschbein et al. (1997) Antisense Nucleic Acid Drug Dev 7:55-61). Merely to illustrate, the backbone of an RNAi construct can be modified with phosphorothioates, phosphoramidate, phosphodithioates, chimeric methylphosphonate-phosphodiesters, peptide nucleic acids, 5-propynyl-pyrimidine containing oligomers or sugar modifications (e.g., 2′-substituted ribonucleosides, a-configuration).

The double-stranded structure may be formed by a single self-complementary RNA strand or two complementary RNA strands. RNA duplex formation may be initiated either inside or outside the cell. The RNA may be introduced in an amount which allows delivery of at least one copy per cell. Higher doses (e.g., at least 5, 10, 100, 500 or 1000 copies per cell) of double-stranded material may yield more effective inhibition, while lower doses may also be useful for specific applications. Inhibition is sequence-specific in that nucleotide sequences corresponding to the duplex region of the RNA are targeted for genetic inhibition.

In certain embodiments, the subject RNAi constructs are “small interfering RNAs” or “siRNAs.” These nucleic acids are around 19-30 nucleotides in length, and even more preferably 21-23 nucleotides in length, e.g., corresponding in length to the fragments generated by nuclease “dicing” of longer double-stranded RNAs. The siRNAs are understood to recruit nuclease complexes and guide the complexes to the target mRNA by pairing to the specific sequences. As a result, the target mRNA is degraded by the nucleases in the protein complex. In a particular embodiment, the 21-23 nucleotides siRNA molecules comprise a 3′ hydroxyl group.

The siRNA molecules of the present invention can be obtained using a number of techniques known to those of skill in the art. For example, the siRNA can be chemically synthesized or recombinantly produced using methods known in the art. For example, short sense and antisense RNA oligomers can be synthesized and annealed to form double-stranded RNA structures with 2-nucleotide overhangs at each end (Caplen, et al. (2001) Proc Natl Acad Sci USA, 98:9742-9747; Elbashir, et al. (2001) EMBO J, 20:6877-88). These double-stranded siRNA structures can then be directly introduced to cells, either by passive uptake or a delivery system of choice, such as described below.

In certain embodiments, the siRNA constructs can be generated by processing of longer double-stranded RNAs, for example, in the presence of the enzyme dicer. In one embodiment, the Drosophila in vitro system is used. In this embodiment, dsRNA is combined with a soluble extract derived from Drosophila embryo, thereby producing a combination. The combination is maintained under conditions in which the dsRNA is processed to RNA molecules of about 21 to about 23 nucleotides.

The siRNA molecules can be purified using a number of techniques known to those of skill in the art. For example, gel electrophoresis can be used to purify siRNAs. Alternatively, non-denaturing methods, such as non-denaturing column chromatography, can be used to purify the siRNA. In addition, chromatography (e.g., size exclusion chromatography), glycerol gradient centrifugation, affinity purification with antibody can be used to purify siRNAs.

In certain preferred embodiments, at least one strand of the siRNA molecules has a 3′ overhang from about 1 to about 6 nucleotides in length, though may be from 2 to 4 nucleotides in length. More preferably, the 3′ overhangs are 1-3 nucleotides in length. In certain embodiments, one strand having a 3′ overhang and the other strand being blunt-ended or also having an overhang. The length of the overhangs may be the same or different for each strand. In order to further enhance the stability of the siRNA, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′deoxythyinidine is tolerated and does not affect the efficiency of RNAi. The absence of a 2′ hydroxyl significantly enhances the nuclease resistance of the overhang in tissue culture medium and may be beneficial in vivo.

In other embodiments, the RNAi construct is in the form of a long double-stranded RNA. In certain embodiments, the RNAi construct is at least 25, 50, 100, 200, 300 or 400 bases. In certain embodiments, the RNAi construct is 400-800 bases in length. The double-stranded RNAs are digested intracellularly, e.g., to produce siRNA sequences in the cell. However, use of long double-stranded RNAs in vivo is not always practical, presumably because of deleterious effects which may be caused by the sequence-independent dsRNA response. In such embodiments, the use of local delivery systems and/or agents which reduce the effects of interferon or PKR are preferred.

In certain embodiments, the RNAi construct is in the form of a hairpin structure (named as hairpin RNA). The hairpin RNAs can be synthesized exogenously or can be formed by transcribing from RNA polymerase III promoters in vivo. Examples of making and using such hairpin RNAs for gene silencing in mammalian cells are described in, for example, Paddison et al., Genes Dev, 2002, 16:948-58; McCaffrey et al., Nature, 2002, 418:38-9; McManus et al., RNA, 2002, 8:842-50; Yu et al., Proc Natl Acad Sci USA, 2002, 99:6047-52). Preferably, such hairpin RNAs are engineered in cells or in an animal to ensure continuous and stable suppression of a desired gene. It is known in the art that siRNAs can be produced by processing a hairpin RNA in the cell.

In yet other embodiments, a plasmid is used to deliver the double-stranded RNA, e.g., as a transcriptional product. In such embodiments, the plasmid is designed to include a “coding sequence” for each of the sense and antisense strands of the RNAi construct. The coding sequences can be the same sequence, e.g., flanked by inverted promoters, or can be two separate sequences each under transcriptional control of separate promoters. After the coding sequence is transcribed, the complementary RNA transcripts base-pair to form the double-stranded RNA.

PCT application WO01/77350 describes an exemplary vector for bi-directional transcription of a transgene to yield both sense and antisense RNA transcripts of the same transgene in a eukaryotic cell. Accordingly, in certain embodiments, the present invention provides a recombinant vector having the following unique characteristics: it comprises a viral replicon having two overlapping transcription units arranged in an opposing orientation and flanking a transgene for an RNAi construct of interest, wherein the two overlapping transcription units yield both sense and antisense RNA transcripts from the same transgene fragment in a host cell.

Exemplary RNAi constructs that specifically recognize a particular gene, or a particular family of genes can be selected using methodology outlined in detail above with respect to the selection of antisense oligonucleotide. Similarly, methods of delivery of RNAi constructs include the methods for delivery antisense oligonucleotides outlined in detail above.

Ribozyme molecules designed to catalytically cleave an mRNA transcripts can also be used to prevent translation of mRNA (See, e.g., PCT International Publication WO90/11364, published Oct. 4, 1990; Sarver et al., 1990, Science 247:1222-1225 and U.S. Pat. No. 5,093,246). While ribozymes that cleave mRNA at site-specific recognition sequences can be used to destroy particular mRNAs, the use of hammerhead ribozymes is preferred. Hammerhead ribozymes cleave mRNAs at locations dictated by flanking regions that form complementary base pairs with the target mRNA. The sole requirement is that the target mRNA have the following sequence of two bases: 5′-UG-3′. The construction and production of hammerhead ribozymes is well known in the art and is described more fully in Haseloff and Gerlach, 1988, Nature, 334:585-591.

The ribozymes of the present invention also include RNA endoribonucleases (hereinafter “Cech-type ribozymes”) such as the one which occurs naturally in Tetrahymena thermophila (known as the IVS, or L-19 IVS RNA) and which has been extensively described by Thomas Cech and collaborators (Zaug, et al., 1984, Science, 224:574-578; Zaug and Cech, 1986, Science, 231:470475; Zaug, et al., 1986, Nature, 324:429433; published International patent application No. WO88/04300 by University Patents Inc.; Been and Cech, 1986, Cell, 47:207-216). The Cech-type ribozymes have an eight b ase pair active site that hybridizes to a target RNA sequence whereafter cleavage of the target RNA takes place. The invention encompasses those Cech-type ribozymes that target eight base-pair active site sequences.

As in the antisense approach, the ribozymes can be composed of modified oligonucleotides (e.g., for improved stability, targeting, etc.) and can be delivered to cells in vitro or in vivo. A preferred method of delivery involves using a DNA construct “encoding” the ribozyme under the control of a strong constitutive pol III or pol II promoter, so that transfected cells will produce sufficient quantities of the ribozyme to destroy targeted messages and inhibit translation. Because ribozymes, unlike antisense molecules, are catalytic, a lower intracellular concentration is required for efficiency.

DNA enzymes incorporate some of the mechanistic features of both antisense and ribozyme technologies. DNA enzymes are designed so that they recognize a particular target nucleic acid sequence, much like an antisense oligonucleotide, however much like a ribozyme they are catalytic and specifically cleave the target nucleic acid.

There are currently two basic types of DNA enzymes, and both of these were identified by Santoro and Joyce (see, for example, U.S. Pat. No. 6,110,462). The 10-23 DNA enzyme comprises a loop structure which connect two arms. The two arms provide specificity by recognizing the particular target nucleic acid sequence while the loop structure provides catalytic function under physiological conditions.

Briefly, to design an ideal DNA enzyme that specifically recognizes and cleaves a target nucleic acid, one of skill in the art must first identify the unique target sequence. This can be done using the same approach as outlined for antisense oligonucleotides. Preferably, the unique or substantially unique sequence is a G/C rich region of approximately 18 to 22 nucleotides. High G/C content helps insure a stronger interaction between the DNA enzyme and the target sequence.

When synthesizing the DNA enzyme, the specific antisense recognition sequence that will target the enzyme to the message is divided so that it comprises the two arms of the DNA enzyme, and the DNA enzyme loop is placed between the two specific arms. Methods of making and administering DNA enzymes can be found, for example, in U.S. Pat. No. 6,110,462. Similarly, methods of delivering DNA ribozymes in vitro or in vivo include methods of delivering RNA ribozyme, as outlined in detail above. Additionally, one of skill in the art will recognize that, like antisense oligonucleotide, DNA enzymes can be optionally modified to improve stability and improve resistance to degradation.

Antibodies can be used as inhibitors of the activity of a particular protein. Antibodies can have extraordinary affinity and specificity for particular epitopes. Antibodies that bind to a particular protein in such a way that the binding of the antibody to the epitope on the protein can interfere with the function of that protein. For example, an antibody may inhibit the function of the protein by sterically hindering the proper protein-protein interactions or occupying active sites. Alternatively the binding of the antibody to an epitope on the particular protein may alter the conformation of that protein such that it is no longer able to properly function.

Monoclonal or polyclonal antibodies can be made using standard protocols (See, for example, Antibodies: A Laboratory Manual ed. by Harlow and Lane (Cold Spring Harbor Press: 1988)). A mammal, such as a mouse, a hamster, a rat, a goat, or a rabbit can be immunized with an immunogenic form of the peptide. Techniques for conferring immunogenicity on a protein or peptide include conjugation to carriers or other techniques well known in the art.

Following immunization of an animal with an antigenic preparation of a polypeptide, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495497), the human B cell hybridoma technique (Kozbar et al., (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with a particular polypeptide and monoclonal antibodies isolated from a culture comprising such hybridoma cells.

In the context of the present invention, antibodies can be screened and tested to identify those antibodies that can inhibit the function of a particular protein. One of skill in the art will recognize that not every antibody that is specifically immunoreactive with a particular protein will interfere with the function of that protein. However, one of skill in the art can readily test antibodies to identify those that are capable of blocking the function of a particular protein.

The term antibody as used herein is intended to include fragments thereof which are also specifically reactive with a particular polypeptide. Antibodies can be fragmented using conventional techniques and the fragments screened for utility in the same manner as described above for whole antibodies. For example, F(ab)₂ fragments can be generated by treating antibody with pepsin. The resulting F(ab)₂ fragment can be treated to reduce disulfide bridges to produce Fab fragments. The antibody of the present invention is further intended to include bispecific and chimeric molecules having affinity for a particular protein conferred by at least one CDR region of the antibody.

Both monoclonal and polyclonal antibodies (Ab) directed against a particular polypeptides, and antibody fragments such as Fab, F(ab)₂, Fv and scFv can be used to block the action of a particular protein. Such antibodies can be used either in an experimental context to further understand the role of a particular protein in a biological process, or in a therapeutic context.

In addition to the use of antibodies to inhibit the function of a particular protein, the present invention contemplates that antibodies raised against a particular protein can also be used to monitor the expression of that protein in vitro or in vivo (e.g., such antibodies can be used in immunohistochemical staining).

Polypeptides and peptide fragments can either agonize or antagonize the function of a particular protein, and such polypeptides and polypeptide variants can promote elastin signaling, and thereby be useful in methods of influencing one or more property of vascular smooth muscle cells. In some aspects, the polypeptide comprises a bioactive portion of a polypeptide, and expression of that polypeptide in the cell promotes elastin signaling. In other aspects, the polypeptide comprises an antagonistic variant of a wildtype polypeptide, and this antagonistic variant inhibits the expression and/or activity of a protein that inhibits elastin signaling. Such an antagonistic polypeptide could be used to promote elastin signaling by relieving the inhibitory affect of a particular protein.

One of skill in the art can readily make and test wildtype polypeptides, polypeptide variants, and peptide fragments to determine if said polypeptide activates elastin signaling. Examples of such variants and fragments include dominant negative mutants of a particular protein.

One of skill in the art can readily make variants comprising an amino acid sequence at least 60%, 70%, 75%, 80%, 85%, 90%, 95%, 98% or 99% identical to a particular polypeptide, and identify variants that activate elastin signaling by either agonizing or antagonizing the function of the wildtype protein. Further examples of antagonistic variants and antagonistic peptide fragments are described in the present application.

Small organic molecules can agonize or antagonize the function of a particular protein. By small organic molecule is meant a carbon containing molecule having a molecular weight less than 2500 amu, more preferably less than 1500 amu, and even more preferably less than 750 amu. In the context of the present invention, such small organic molecules would be able to promote elastin signaling by (i) binding to elastin and potentiating elastin signaling, (ii) binding to an elastin receptor and potentiating elastin signaling, (iii) promoting expression and/or activity of RhoA, (iv) promoting expression and/or activity of tropoelastin, (v) promoting expression and/or activity of an elastin receptor, (vi) promoting expression and/or activity of a GEF. Further small organic molecules that promote elastin signaling do so by (i) inhibiting expression and/or activity of a GAP, or (ii) inhibiting expression and/or activity of a GDI.

Small organic molecules can be readily identified by screening libraries of organic molecules and/or chemical compounds to identify those compounds that have a desired function. Without being bound by theory, small organic molecules may exert their inhibitory function in any of a number of ways including promoting expression and/or activity of a protein involved in promoting elastin signaling, promoting elastin signal transduction, inhibiting expression and/or activity of a protein which inhibits elastin signaling, or inhibiting elastin signal transduction.

In addition to screening readily available libraries to identify small organic molecules with a particular function, the present invention contemplates the rational design and testing of small organic molecules that promotes elastin signaling. For example, based on molecular modeling of the binding site of a particular protein, one of skill in the art can design small molecules that can occupy that binding pocket. Such small organic molecules would be candidate inhibitors of the function of that particular protein. Further rational design can be based on analysis of the ligand binding domain of a particular receptor, the DNA binding domain of a transcription factor, or a cofactor binding domain of a receptor or ligand.

The present invention contemplates a large number of agents that promote elastin signaling including nucleic acids, peptides, polypeptides, peptidomimetics, small organic molecules, antisense oligonucleotides, RNAi constructs, antibodies, ribozymes, and DNA enzymes. Exemplary agents include both agents that positively regulate proteins involved in elastin signaling, as well as agents that negatively regulate proteins that inhibit elastin signaling. Furthermore, agents for use in the methods of the present invention include agents which potentiate elastin signaling, even when said agent acts via an unknown mechanism.

Agents that promote elastin signaling, either in vivo or in vitro, and can be used in the methods of the present invention have one or more of the following functions: (i) increase actin stress fiber formation in vascular smooth muscle cells, (ii) increase expression of v inculin in vascular smooth muscle cells, (iii) increase focal adhesion formation in vascular smooth muscle cells, (iv) inhibit dedifferentiation of vascular smooth muscle cells, (v) promote actin polymerization in vascular smooth muscle cells, (vi) increase the ratio of F:G actin in vascular smooth muscle cells, (vii) decrease or inhibit occlusion of a vessel, (viii) decrease or inhibit vascular obstruction, (ix) decrease or inhibit restenosis, and/or (x) prevent stenosis.

(b) Exemplary Compositions

The present invention contemplates the use of any of a number of agents for promoting elastin signaling. Agents for use in the methods of the present invention promote elastin signaling and have one or more of the following functions: (i) increase actin stress fiber formation in vascular smooth muscle cells, (ii) increase expression of vinculin in vascular smooth muscle cells, (iii) increase focal adhesion formation in vascular smooth muscle cells, (iv) inhibit dedifferentiation of vascular smooth muscle cells, (v) promote actin polymerization in vascular smooth muscle cells, (vi) increase the ratio of F:G actin in vascular smooth muscle cells, (vii) decrease or inhibit occlusion of a vessel, (viii) decrease or inhibit vascular obstruction, (ix) decrease or inhibit restenosis, and/or (x) prevent stenosis.

Agents that promote elastin signaling in vascular smooth muscle cells or in smooth muscle cells include, but are not limited to tropoelastin (SEQ ID NO: 2) and bioactive fragments thereof (SEQ ID NO: 3). Based on our understanding of the elastin signaling pathway, the invention contemplates that further exemplary agents include the elastin receptor, a constituitively active elastin receptor, agents that activate Gi, RhoA GTPase (SEQ ID NO: 6), and a constituitively active RhoA GTPase.

In light of the understanding in the art of the regulation of RhoA, the present application further contemplates that GEFs, GAPs and GDIs can function as activators of elastin signaling. GEFs promote the “on” state of Rho family GTPases, and thus a nucleic acid sequence encoding a GEF polypeptide or a polypeptide comprising a GEF amino acid sequence can be used to activate elastin signaling. Additionally, agents which increase the expression or activity of a GEF can activate elastin signaling. GAPs promote the “off” state of Rho family GTPases. Agents which decrease the expression or activity of a GAP activate elastin signaling. GDIs promote the “off” state of Rho family GTPases. Agents which decrease the expression or activity of a GDI activate elastin signaling.

Without being bound by theory, an agent that activates or potentiates elastin signaling may function in any of a number of ways. Exemplary agents include, but are not limited to (a) nucleic acids encoding tropoelastin, (b) nucleic acids encoding a bioactive fragment of tropoelastin, (c) nucleic acids encoding the elastin receptor, (d) nucleic acids encoding a constitutively elastin receptor, (e) nucleic acids encoding Gi heterotrimeric G protein, (f) nucleic acids encoding a constitutively active Gi heterotrimeric G protein, (g) nucleic acids encoding a RhoA GTPase, (h) nucleic acids encoding an constitutively activate RhoA GTPase, (i) polypeptides comprising an amino acid sequence of tropoelastin, (j) polypeptides comprising an amino acid sequence of a bioactive fragment of tropoelastin, (k) polypeptides comprising an amino acid sequence of the elastin receptor, (l) polypeptides comprising an amino acid sequence of a constitutively active elastin receptor, (m) polypeptides comprising an amino acid sequence of a Gi heterotrimeric G protein, (n) polypeptides comprising an amino acid sequence of a constitutively active Gi heterotrimeric G protein, (O) polypeptides comprising an amino acid sequence of a RhoA GTPase, (p) polypeptides comprising an amino acid sequence of a constitutively active RhoA GTPase, (q) polypeptides comprising an amino acid sequences that bind to and activate the elastin receptor in the presence or absence of ligand, (r) small organic molecules that increase the expression and/or activity of Gi, (s) small organic molecules that increase the expression and/or activity of the elastin receptor, (t) small organic molecules that bind to and activate the elastin receptor in the presence or absence of ligand, (u) small organic molecules that bind to tropoelastin and increase the affinity of elastin for the elastin receptor, (v) small organic molecules that bind to the elastin receptor and increase the affinity of elastin for the elastin receptor, (w) small organic molecule that increases the activity and/or expression of RhoA, (x) small organic molecules which increase the “on” state of RhoA, (y) antisense oligonucleotides which decrease the expression of a GAP which inhibits the activity of RhoA, (z) RNAi oligonucleotides or constructs which decrease the expression of a GAP which inhibits the activity of RhoA, (aa) antibodies which bind to and inhibit the activity of a GAP which inhibits the activity of RhoA, (bb) ribozymes which bind to and inhibit the activity of a GAP which inhibits the activity of RhoA, (cc) nucleic acids encoding a GEF which promotes the “on” state of a RhoA, (dd) polypeptides comprising an amino acid sequence of a GEF which promotes the “on” state of a RhoA, (ee) small organic molecules which promote the expression and/or activity of a GEF which promotes the “on” state of RhoA, (ff) small organic molecules which bind to and promote the expression and/or activity of a GEF which promotes the “on” state of RhoA, (gg) antisense oligonucleotides that decrease the expression of a GDI which promotes the “off” state of RhoA, (hh) RNAi constructs that decrease the expression of a GDI which promotes the “off” state of RhoA, (ii) ribozymes that decrease the expression of a GDI which promotes the “off” state of RhoA, (jj) antibodies that decrease the expression and/or activity of a GDI which promotes the “off” state of RhoA, (kk) small organic molecules that decrease the expression and/or activity of a GDI which promotes the “off” state of RhoA, (ll) small organic molecules that bind to and decrease the expression and/or activity of a GDI which promotes the “off” state of RhoA.

The foregoing mechanisms are by no means exhaustive. Agents for use in the subject methods either promote the expression and/or activation of the elastin receptor; promote the expression and/or activation of Gi, promote the expression and/or “on” state of RhoA, increase the expression and/or activity of a protein which promotes the “on” state of RhoA, or decrease the expression and/or activity of a protein which promotes the “off” state of RhoA.

In any of the foregoing, the application contemplates that agents that promote elastin signaling in smooth muscle cells may be administered alone, or may be administered in combination with one or more other agents. Similarly, in methods of screening for additional agents that promote elastin signaling, the application contemplates that agents may be screened singly or in combination with one or more other agents.

Combinations of agents include, but are not limited to (a) two or more agents that promote elastin signaling via the same mechanism; (b) two or more agents that promote elastin signaling via different mechanism; (c) one or more agents that promote elastin signaling and one or more agents that modulate vascular smooth muscle cell proliferation. As described herein, one aspect of the invention pertains to isolated nucleic acids comprising nucleotide sequences agents that promote elastin signaling in smooth muscle cells, and/or equivalents of such nucleic acids. Exemplary agents include tropoelastin, bioactive fragments of tropoelastin, elastin receptor, constitutively activate elastin receptor, Gi, constitutively activate Gi, RhoA, constitutively activate RhoA. Further exemplary agents include nucleic acids encoding agents which promote the “on” state of RhoA and nucleic acids encoding agents which activate Gi. The term nucleic acid as used herein is intended to include fragments as equivalents, wherein such fragments have substantially the same function as the full length nucleic acid sequence from which it is derived. The term equivalent is understood to include nucleotide sequences encoding agents which are functionally equivalent to the activators of elastin signaling disclosed herein, as well as agents having substantially the same function as the activators of elastin signaling disclosed herein. Equivalent nucleotide sequences will include sequences that differ by one or more nucleotide substitutions, additions or deletions, such as allelic variants; and will, therefore, include sequences that differ from the nucleotide sequence of, for example, the wildtype tropoelastin (SEQ ID NO:1) or RhoA (SEQ ID NO:5) sequence. Equivalent sequences include those that vary from a known wildtype or variant sequence due to the degeneracy of the genetic code. Equivalent sequences may also include nucleotide sequences that hybridize under stringent conditions (i.e., equivalent to about 20-27° C. below the melting temperature (Tₘ) of the DNA duplex formed in about 1 M salt) to the nucleotide sequence of an agent that promotes elastin signaling. Further examples of stringent hybridization conditions include a wash step of 0.2×SSC at 65° C. For the foregoing examples of equivalents to the elastin activators for use in the methods of the present invention, one of skill in the art will recognize that an equivalent sequence retains the function of the particular elastin activator.

In one example, the invention contemplates a method of promoting elastin signaling by administering an agent that promotes elastin signaling in smooth muscle cells, wherein the agent comprises a nucleic acid sequence which hybridizes under stringent conditions, including a wash step of 0.2×SSC at 65° C., to a nucleic acid sequence of SEQ ID NO: 1 or SEQ ID NO: 5.

Equivalent nucleotide sequences for use in the methods described herein also include sequences which are at least 60% identical to the nucleotide sequence of a given agent that promotes elastin signaling. In another embodiment, the nucleotide sequence is at least 70%, 75%, 80%, 85%, 90%, 95%, 98%, 99%, or 100% identical to the nucleotide sequence of a given agent that promotes elastin signaling. In the foregoing, one of skill in the art will recognize that equivalent sequences at least 60%, 70%, 75%, 80%, 85%, 90%, 95%, 98%, 99%, or 100% identical to an agent that promotes elastin signaling in smooth muscle cells and which retain the function of the agent, are useful in the methods of the present invention. In one embodiment, the invention contemplates a method of promoting elastin signaling by administering an agent, wherein the agent comprises a nucleic acid sequence 60%, 70%, 75%, 80%, 85%, 90%, 95%, 98%, 99% or 100% identical to the nucleic acid sequence of, for example, SEQ ID NO: 1 or SEQ ID NO: 5.

Moreover, it will be generally appreciated that, under certain circumstances, it may be advantageous to provide a variant of an agent that promotes elastin signaling that functions as an antagonist (e.g, inhibits elastin signaling in smooth muscle cells). An antagonistic variant may be useful to regulate the rate or extent of elastin signaling, or to limit the effects of a particular agent on smooth muscle cells.

Nucleic acids having a sequence that differs from nucleotide sequences which encode a particular agent that promotes elastin signaling due to degeneracy in the genetic code are also within the scope of the invention. Such nucleic acids encode functionally equivalent peptides but differ in sequence from wildtype sequences known in the art due to degeneracy in the genetic code. For example, a number of amino acids are designated by more than one triplet. Codons that specify the same amino acid, or synonyms (for example, CAU and CAC each encode histidine) may result in “silent” mutations which do not affect the amino acid sequence. However, it is expected that DNA sequence polymorphisms that do lead to changes in the amino acid sequences will also exist. One skilled in the art will appreciate that these variations in one or more nucleotides (up to about 3-5% of the nucleotides) of the nucleic acids encoding polypeptides having a functional activity of elastin activator may exist among individuals of a given species due to natural allelic variation.

In general, polypeptides referred to herein as having an activity of an elastin activator polypeptide (e.g., “bioactive polypeptides”) are defined as polypeptides which include an amino acid sequence corresponding (e.g., at least 80%, 85%, 90%, 95%, 98%, 100% identical) to all or a portion of an amino acid sequence of a wildtype elastin activator polypeptide. Exemplary agents include, but are not limited to, tropoelastin, elastin receptor, constitutively elastin receptor, RhoA, constitutively RhoA, Gi, activated Gi, GEF, etc. Bioactive polypeptides, or bioactive fragments of said polypeptides, will have an activity of an elastin activator. In one embodiment, the invention contemplates a method of promoting elastin signaling in smooth muscle cells comprising administering an elastin activator, wherein the elastin activator comprises an amino acid sequence at least 80%, 85%, 90%, 95%, 98%, 99%, or 100% identical to SEQ ID NO: 2, SEQ ID NO: 3, or SEQ ID NO: 6.

It is further appreciated that agents for use in the methods of the invention may comprise polypeptides encodable by a nucleic acid sequence. In one embodiment, the invention contemplates a method of promoting elastin signaling in smooth muscle cells by administering a elastin activator, wherein the elastin activator comprises an amino acid sequence encodable by a nucleic acid sequence which hybridizes under stringent conditions, including a wash step of 0.2×SSC at 65° C., to a nucleic acid sequence of SEQ ID NO: 1 or SEQ ID NO: 5.

Moreover, it will be appreciated that, under certain circumstances, it may be advantageous to provide a polypeptide which is a variant of a particular agent that promotes elastin signaling, and that functions as an antagonist (e.g, inhibits the function of an agents that promotes elastin signaling).

(iv) Methods of Expressing an Agent that Activates Elastin Signaling

The systems and methods described herein also provide expression vectors containing a nucleic acid encoding an agent that activates elastin signaling operably linked to at least one transcriptional regulatory sequence. Exemplary nucleic acids encoding an agent that activates elastin signaling include, but are not limited to, a nucleic acid encoding tropoelastin, a nucleic acid encoding an elastin fragment that activates elastin signaling, a nucleic acid encoding a RhoA GTPase, a nucleic acid encoding an activated RhoA GTPase, a nucleic acid encoding a protein that activates a RhoA GTPase, a nucleic acid encoding an elastin receptor, and a nucleic acid encoding an activator of Gi.

Regulatory sequences are art-recognized and are selected to direct expression of the subject proteins. Accordingly, the term transcriptional regulatory sequence includes promoters, enhancers and other expression control elements. Such regulatory sequences are described in Goeddel; Gene Expression Technology: Methods in Enzymology 185, Academic Press, San Diego, Calif. (1990). For instance, any of a wide variety of expression control sequences may be used in these vectors to express nucleic acid sequences encoding the agents of this invention. Such useful expression control sequences, include, for example, a viral LTR, such as the LTR of the Moloney murine leukemia virus, the LTR of the Herpes Simplex virus-1, the early and late promoters of SV40, adenovirus or cytomegalovirus immediate early promoter, the lac system, the trp system, the TAC or TRC system, T7 promoter whose expression is directed by T7 RNA polymerase, the major operator and promoter regions of phage λ, the control regions for fd coat protein, the promoter for 3-phosphoglycerate kinase or other glycolytic enzymes, the promoters of acid phosphatase, the promoters of the yeast α-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of protein desired to be expressed. Moreover, the vector's copy number, the ability to control that copy number and the expression of any other proteins encoded by the vector, such as antibiotic markers, should also be considered.

Moreover, the gene constructs can be used to deliver nucleic acids encoding the subject polypeptides. Thus, another aspect of the invention features expression vectors for in vivo or in vitro transfection, viral infection and expression of a subject polypeptide in particular cell types.

Expression constructs of the subject agents may be administered in biologically effective carriers, e.g. any formulation or composition capable of effectively delivering the recombinant gene to cells in vivo or in vitro. Approaches include insertion of the subject gene in viral vectors including recombinant retroviruses, adenovirus, adeno-associated virus, herpes simplex virus-1, lentivirus, mammalian baculovirus or recombinant bacterial or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct, electroporation or CaPO₄ precipitation. One of skill in the art c an readily select from available vectors and methods of delivery in order to optimize expression in a particular cell type or under particular conditions.

Retrovirus vectors and adeno-associated virus vectors have been frequently used for the transfer of exogenous genes. These vectors provide efficient delivery of genes into cells, and the transferred nucleic acids are stably integrated into the chromosomal DNA of the host. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses for gene therapy, and defective retroviruses are well characterized for use in gene transfer for gene therapy purposes. Thus, recombinant retrovirus can be constructed in which part of the retroviral coding sequence (gag, pol, env) has been replaced by nucleic acid encoding one of the subject proteins rendering the retrovirus replication defective. The replication defective retrovirus is then packaged into virions through the use of a helper virus by standard techniques which can be used to infect a target cell. Protocols for producing recombinant retroviruses and for infecting cells in vitro or in vivo with such viruses can be found in Current Protocols in Molecular Biology, Ausubel, F. M. et al. (eds.) Greene Publishing Associates, (2000), and other standard laboratory manuals. Examples of suitable retroviruses include pBPSTR1, pLJ, pZIP, pWE and pEM which are known to those skilled in the art. Examples of suitable packaging virus lines for preparing both ecotropic and amphotropic retroviral systems include ψCrip, ψCre, ψ2, ψAm, and PA317.

Furthermore, it has been shown that it is possible to limit the infection spectrum of retroviruses and consequently of retroviral-based vectors, by modifying the viral packaging proteins on the surface of the viral particle (see, for example PCT publications WO93/25234 and WO94/06920). For instance, strategies for the modification of the infection spectrum of retroviral vectors include: coupling antibodies specific for cell surface antigens to the viral env protein; or coupling cell surface receptor ligands to the viral env proteins. Coupling can be in the form of the chemical cross-linking with a protein or other variety (e.g. lactose to convert the env protein to an asialoglycoprotein), as well as by generating fusion proteins (e.g. single-chain antibody/env fusion proteins). This technique, while useful to limit or otherwise direct the infection to certain tissue types, can also be used to convert an ecotropic vector into an amphotropic vector.

Moreover, use of retroviral gene delivery can be further enhanced by the use of tissue- or cell-specific transcriptional regulatory sequences which control expression of the gene of the retroviral vector such as tetracycline repression or activation.

Another viral gene delivery system which has been employed utilizes adenovirus-derived vectors. The genome of an adenovirus can be manipulated so that it encodes and expresses a gene product of interest but is inactivated in terms of its ability to replicate in a normal lytic viral life cycle. Suitable adenoviral vectors derived from the adenovirus strain Ad type 5 dl324 or other strains of adenovirus (e.g., Ad2, Ad3, Ad7 etc.) are known to those skilled in the art. Recombinant adenoviruses can be advantageous in certain circumstances in that they can be used to infect a wide variety of cell types, including airway epithelium, endothelial cells, hepatocytes, and muscle cells. Furthermore, the virus particle is relatively stable and amenable to purification and concentration, and as above, can be modified so as to affect the spectrum of infectivity.

Yet another viral vector system is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al. Curr. Topics in Micro. and Immunol. (1992) 158: 97-129). It is also one of the few viruses that may integrate its DNA into non-dividing cells, and exhibits a high frequency of stable integration.

Another viral delivery system is based on herpes simplex-1 (HSV-1). HSV-1 based vectors have been shown to infect a variety of cells including post mitotic cells such as neuronal cells (Agudo et al. (2002) Human Gene Therapy 13: 665-674; Latchman (2001) Neuroscientist 7: 528-537; Goss et al. (2002) Diabetes 51: 2227-2232; Glorioso (2002) Current Opin Drug Discov Devel 5: 289-295; Evans (2002) Clin Infect Dis 35: 597-605; Whitley (2002) Journal of Clinical Invest 110: 145-151; Lilley (2001) Curr Gene Ther 1: 339-359).

The above cited examples of viral vectors are by no means exhaustive. However, they are provided to indicate that one of skill in the art may select from well known viral vectors, and select a suitable vector for expressing a particular protein in a particular cell type.

In addition to viral transfer methods, such as those illustrated above, non-viral methods can be used to express a subject polypeptide. Many nonviral methods of gene transfer rely on normal mechanisms used by cells for the uptake and intracellular transport of macromolecules. Exemplary gene delivery systems of this type include liposomal derived systems, poly-lysine conjugates, and artificial viral envelopes.

It may sometimes be desirable to introduce a nucleic acid directly to a cell, for example a cell in culture or a cell in an animal. Such administration can be done by injection of the nucleic acid (e.g., DNA, RNA) directly at the desired site. Such methods are commonly used in the vaccine field, specifically for administration of “DNA vaccines”, and include condensed DNA (U.S. Pat. No. 6,281,005).

In addition to administration of nucleic acids, the systems and methods described herein contemplate that polypeptides may be administered directly. Some proteins, for example factors that act extracellularly by contacting a cell surface receptor, such as growth factors, may be a dministered by simply contacting cells with said protein. For example, cells are typically cultured in media which is supplemented by a number of proteins such as FGF, TGFβ, insulin, etc. These proteins influence cells by simply contacting the cells. The current invention contemplates that agents that potentiate elastin signaling can act by simply contacting cells. Such a method similarly pertains to other agents such as small organic molecules and chemical compounds. These agents may either exert their effect at the cell surface, or may be able to permeate the cell membrane without the need for additional manipulation.

In another embodiment, an agent that potentiates elastin signaling is a polypeptide and the polypeptide is directly introduced into a cell. Methods of directly introducing a polypeptide into a cell include, but are not limited to, protein transduction and protein therapy. For example, a protein transduction domain (PTD) can be fused to a nucleic acid encoding a particular agent, and the fusion protein is expressed and purified. Fusion proteins containing the PTD are permeable to the cell membrane, and thus cells can be directly contacted with a fusion protein (Derossi et al. (1994) Journal of Biological Chemistry 269: 10444-10450; Han et al. (2000) Molecules and Cells 6: 728-732; Hall et al. (1996) Current Biology 6: 580-587; Theodore et al. (1995) Journal of Neuroscience 15: 7158-7167).

Although some protein transduction based methods rely on fusion of a polypeptide of interest to a sequence which mediates introduction of the protein into a cell, other protein transduction methods do not require covalent linkage of a protein of interest to a transduction domain. At least two commercially available reagents exist that mediate protein transduction without covalent modification of the protein (Chariot™, produced by Active Motif, www.activemotif.com and Bioporter® Protein Delivery Reagent, produced by Gene Therapy Systems, www.genetherapysystems.com).

Briefly, these protein transduction reagents can be used to deliver proteins, peptides and antibodies directly to cells including mammalian cells. Delivery of proteins directly to cells has a number of advantages. Firstly, many current techniques of gene delivery are based on delivery of a nucleic acid sequence which must be transcribed and/or translated by a cell before expression of the protein is achieved. This results in a time lag between delivery of the nucleic acid and expression of the protein. Direct delivery of a protein decreases this delay. Secondly, delivery of a protein often results in transient expression of the protein in a cell.

As outlined herein, protein transduction mediated by covalent attachment of a PTD to a protein can be used to deliver a protein to a cell. These methods require that individual proteins be covalently appended with PTD moieties. In contrast, methods such as Chariot™ and Bioporter® facilitate transduction by forming a noncovalent interaction between the reagent and the protein. Without being bound by theory, these reagents are thought to facilitate transit of the cell membrane, and following internalization into a cell the reagent and protein complex disassociates so that the protein is free to function in the cell.

In another aspect, this application includes agents that potentiate elastin signaling, wherein the agent is a protein that potentiates elastin signaling. Recombinant polypeptides preferred by the present invention include, but are not limited to, tropoelastin (SEQ ID NO: 2), elastin receptor, RhoA (SEQ ID NO: 6), constitutively active RhoA, a protein that activate Gi, a constitutively active elastin receptor, a GEF, a protein that activates RhoA, and a protein that inhibits the expression and/or activity of an inhibitor of RhoA activation (e.g., a protein that inhibits expression and/or activity of a GAP or GDI). The invention further contemplates the use of variants of such proteins that potentiate elastin signaling, wherein the variant maintains the ability to potentiate elastin signaling. Exemplary variants are at least 60% identical, more preferably 70% identical and most preferably 80% identical with an amino acid sequence of a protein that potentiates elastin signaling. Additional preferred embodiments include recombinant polypeptides comprising an amino acid sequence at least 85%, 90%, 95%, 98%, or 99% identical to an amino acid sequence of a protein that potentiates elastin signaling. The invention further contemplates the use of bioactive fragments of any of the above referenced protein for use in methods of potentiating elastin signaling.

Exemplary bioactive fragments include bioactive fragments of tropoelastin. As detailed herein, one such bioactive fragment is represented in SEQ ID NO: 3.

Additional preferred polypeptide comprise an amino acid sequence at least 80%, 85%, 90%, 95%, 98% or 100% identical to a bioactive fragment of a protein that potentiates elastin signaling. Any of the foregoing polypeptides comprising all or a bioactive portion of a protein that potentiates elastin signaling may be further characterized by one of more of the following functional attributes: (i) increase actin stress fiber formation in vascular smooth muscle cells, (ii) increase expression o f vinculin in vascular smooth muscle cells, (iii) increase focal adhesion formation in vascular smooth muscle cells, (iv) inhibit dedifferentiation of vascular smooth muscle cells, (v) promote actin polymerization in vascular smooth muscle cells, (vi) increase the ratio of F:G a ctin in vascular smooth muscle cells, (vii) decrease or inhibit occlusion of a vessel, (viii) decrease or inhibit vascular obstruction, (ix) decrease or inhibit restenosis, and/or (x) prevent stenosis.

In one example, the application provides a method of promoting actin polymerization in vascular smooth muscle cells, comprising administering an agent that promotes elastin signaling, wherein the agent that promotes elastin signaling is a polypeptide that comprises an amino acid sequence at least 60%, 70%, 80%, 85%, 90%, 95%, 98%, 99%, or 100% identical to SEQ ID NO: 2, SEQ ID NO: 3, or SEQ ID NO: 6. In another embodiment, the application provides a method of decreasing vascular obstruction, comprising administering an agent that promotes elastin signaling, wherein the agent that promotes elastin signaling is a polypeptide that comprises an amino acid sequence at least 60%, 70%, 80%, 85%, 90%, 95%, 98%, 99%, or 100% identical to SEQ ID NO: 2, SEQ ID NO: 3, or SEQ ID NO: 6.

This application also describes methods for producing the subject polypeptides. For example, a host cell transfected with a nucleic acid vector directing expression of a nucleotide sequence encoding the subject polypeptides can be cultured under appropriate conditions to allow expression of the peptide to occur. The polypeptide may be secreted and isolated from a mixture of cells and medium containing the recombinant polypeptide. Alternatively, the peptide may be expressed cytoplasmically and the cells harvested, lysed and the protein isolated. A cell culture includes host cells, media and other by-products. Suitable media for cell culture are well known in the art. The recombinant polypeptide can be isolated from cell culture medium, host cells, or both using techniques known in the art for purifying proteins including ion-exchange chromatography, gel filtration chromatography, ultrafiltration, electrophoresis, and immunoaffinity purification with antibodies specific for such peptide. In one example, the recombinant polypeptide is a fusion protein containing a domain which facilitates its purification, such as a GST fusion protein. In another example, the subject recombinant polypeptide may include one or more additional domains which facilitate immunodetection, purification, and the like. Exemplary domains include HA, FLAG, GST, His, and the like. Further exemplary domains include a protein transduction domain (PTD) which facilitates the uptake of proteins by cells.

This application also describes a host cell which expresses a recombinant form of the subject polypeptides. The host cell may be a prokaryotic or eukaryotic cell. Thus, a nucleotide sequence derived from the cloning of a protein encoding all or a selected portion (either an antagonistic portion or a bioactive fragment) of the full-length protein, can be used to produce a recombinant form of a polypeptide via microbial or eukaryotic cellular processes. Ligating the polynucleotide sequence into a gene construct, such as an expression vector, and transforming or transfecting into hosts, either eukaryotic (yeast, avian, insect or mammalian) or prokaryotic (bacterial cells), are standard procedures used in producing other well-known proteins, e.g. insulin, interferons, human growth hormone, IL-1, IL-2, and the like. Similar procedures, or modifications thereof, can be employed to prepare recombinant polypeptides by microbial means or tissue-culture technology in accord with the subject invention. Such methods are used to produce experimentally useful proteins that include all or a portion of the subject nucleic acids. For example, such methods are used to produce fusion proteins including domains which facilitate purification or immunodetection, and to produce recombinant mutant forms of a protein (for example a dominant negative or constituitively activate form of a Rho family GTPase or a tropoelastin fragment).

The recombinant genes can be produced by ligating a nucleic acid encoding a protein, or a portion thereof, into a vector suitable for expression in either prokaryotic cells, eukaryotic cells, or both. Expression vectors for production of recombinant forms of the subject polypeptides include plasmids and other vectors.

For instance, suitable vectors for the expression of a polypeptide include plasmids of the types: pBR322-derived plasmids, pEMBL-derived plasmids, pEX-derived plasmids, pGEX-derived plasmids, pTrc-His-derived plasmids, pBTac-derived plasmids and pUC-derived plasmids for expression in prokaryotic cells, such as E. coli.

A number of vectors exist for the expression of recombinant proteins in yeast For instance, YEP24, YIPS5, YEP51, YEP52, pYES2, and YRP17 are cloning and expression vehicles useful in the introduction of genetic constructs into S. cerevisiae.

Many mammalian expression vectors contain both prokaryotic sequences, to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSV7, pko-neo, pBacMam-2, and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. For other suitable expression systems for both prokaryotic and eukaryotic cells, as well as general recombinant procedures, see Molecular Cloning A Laboratory Manual, 3rd Ed., ed. by Sambrook and Russell (Cold Spring Harbor Laboratory Press: 2001).

In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the β-gal containing pBlueBac III).

When it is desirable to express only a portion of a protein, such as a form lacking a portion of the N-terminus, e.g. a truncation mutant, it may be necessary to add a start codon (ATG) to the oligonucleotide fragment containing the desired sequence to be expressed. It is well known in the art that a methionine at the N-terminal position can be enzymatically cleaved by the enzyme methionine aminopeptidase (MAP).

Techniques for making fusion genes are known to those skilled in the art. The joining of various nucleic acid fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another example, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence.

The present invention also makes available isolated polypeptides which are isolated from, or otherwise substantially free of other cellular and extracellular proteins. The term “substantially free of other cellular or extracellular proteins” (also referred to herein as “contaminating proteins”) or “substantially pure or purified preparations” are defined as encompassing preparations having less than 20% (by dry weight) contaminating protein, and preferably having less than 5% contaminating protein. Functional forms of the subject proteins can be prepared as purified preparations by using a cloned gene as described herein. By “purified”, it is meant, when referring to peptide or nucleic acid sequences, that the indicated molecule is present in the substantial absence of other biological macromolecules, such as other proteins. The term “purified” as used herein preferably means at least 80% by dry weight, more preferably in the range of 95-99% by weight, and most preferably at least 99.8% by weight, of biological macromolecules of the same type present (but water and buffers can be present). The term “pure” as used herein preferably has the same numerical limits as “purified” immediately above. “Isolated” and “purified” do not encompass either natural materials in their native state or natural materials that have been separated into components (e.g., in an acrylamide gel) but not obtained either as pure (e.g. lacking contaminating proteins, or chromatography reagents such as denaturing agents and polymers, e.g. acrylamide or agarose) substances or solutions.

Isolated peptidyl portions of proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry.

The recombinant polypeptides of the present invention also include versions of those proteins that are resistant to proteolytic cleavage. Variants of the present invention also include proteins which have been post-translationally modified in a manner different than the authentic protein. Modification of the structure of the subject polypeptides can be for such purposes as enhancing therapeutic or prophylactic efficacy, or stability (e.g., ex vivo shelf life and resistance to proteolytic degradation in vivo). Such modified peptides, when designed to retain at least one activity of the naturally-occurring form of the protein, are considered functional equivalents of the polypeptides described in more detail herein. Such modified peptides can be produced, for instance, by amino acid substitution, deletion, or addition.

For example, it is reasonable to expect that, in some instances, an isolated replacement of a leucine with an isoleucine or valine, an aspartate with a glutamate, a threonine with a serine, or a similar replacement of an amino acid with a structurally related amino acid (e.g., isosteric and/or isoelectric mutations) may not have a major effect on the biological activity of the resulting molecule. Conservative replacements are those that take place within a family of amino acids that are related in their side chains. Genetically encoded amino acids can be divided into four families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. Phenylalanine, tryptophan, and tyrosine are sometimes classified jointly as aromatic amino acids. In similar fashion, the amino acid repertoire can be grouped as (1) acidic=a spartate, glutamate; (2) basic=lysine, arginine histidine, (3) aliphatic=glycine, alanine, valine, leucine, isoleucine, serine, threonine, with serine and threonine optionally be grouped separately as aliphatic-hydroxyl; (4) aromatic=phenylalanine, tyrosine, tryptophan; (5) amide=asparagine, glutamine; and (6) sulfur-containing=cysteine and methionine. (see, for example, Biochemistry, 5th ed. by Berg, Tymoczko and Stryer, WH Freeman and Co.: 2002). Whether a change in the amino acid sequence of a peptide results in a functional variant (e.g. functional in the sense that it acts to mimic or antagonize the wild-type form) can be determined by assessing the ability of the variant peptide to produce a response in cells in a fashion similar to the wild-type protein, or competitively inhibit such a response. Polypeptides in which more than one replacement has taken place can readily be tested in the same manner.

Advances in the fields of combinatorial chemistry and combinatorial mutagenesis have facilitated the making of polypeptide variants (Wissmanm et al. (1991) Genetics 128: 225-232; Graham et al. (1993) Biochemistry 32: 6250-6258; York et al. (1991) Journal of Biological Chemistry 266: 8495-8500; Reidhaar-Olson et al. (1988) Science 241: 53-57). Given one or more assays for testing polypeptide variants, one can assess whether a given variant functions to potentiate elastin signaling, or whether a given variant has an antagonistic function. In the context of the present invention, several methods for assaying the functional activity of potential activators of elastin signaling are provided. Activity can be accessed in vitro, for example, in the culture systems provided in the examples. Furthermore, the function of a polypeptide variant can be examined in cells and cell lines such as smooth muscle cells and vascular smooth muscle cells.

To further illustrate, the invention contemplates a method for generating sets of combinatorial mutants, as well as truncation mutants, and is especially useful for identifying potential variant sequences that maintain the ability to activate elastin signaling. Such a method is similarly useful for identifying potential variant sequences that antagonize the function of a wildtype elastin activator. The purpose of screening such combinatorial libraries is to generate, for example, novel variants which can act as either agonists or antagonists, or alternatively, possess novel activities all together.

In one aspect of this method, the amino acid sequences for a population of elastin activators are aligned, preferably to promote the highest homology possible. By a population of proteins is meant the alignment of, for example, proteins that activate elastin signaling from several different species (e.g., human, mouse, rat, cow, etc.). By way of example, one o f skill in the art can align the amino acid sequences of tropoelastin from several different species or the amino acid sequences from RhoA from several different species.

Amino acids which appear at each position of the aligned sequences are selected to create a degenerate set of combinatorial sequences. In one example, the variegated library of variants is generated by combinatorial mutagenesis at the nucleic acid level, and is encoded by a variegated gene library. For instance, a mixture of synthetic oligonucleotides can be enzymatically ligated into gene sequences such that the degenerate set of potential sequences are expressible as individual polypeptides, or alternatively, as a set of larger fusion proteins (e.g. for phage display) containing the set of sequences therein.

The library of potential variants can be generated from a degenerate oligonucleotide sequence using a variety of methods. Chemical synthesis of a degenerate gene sequence can be carried out in an automatic DNA synthesizer, and the synthetic genes then ligated into an appropriate expression vector. One purpose of a degenerate set of genes is to provide, in one mixture, all the sequences encoding the desired set of potential variant sequences. The synthesis of degenerate oligonucleotides is known in the art.

A range of techniques are known for screening gene products of combinatorial libraries made by point mutations, and for screening cDNA libraries for gene products having a certain property. Such techniques will be generally adaptable for rapid screening of the gene libraries generated by the combinatorial mutagenesis of agents that potentiate elastin signaling. These techniques are also applicable for rapid screening of other gene libraries. One example of the techniques used for screening large gene libraries includes cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

The application also describes reducing a protein to generate mimetics, e.g. peptide or non-peptide agents. Such mimetics promote elastin signaling and possess one or more of the following functional characteristics including, but not limited to: (i) increase actin stress fiber formation in vascular smooth muscle cells, (ii) increase expression of vinculin in vascular smooth muscle cells, (iii) increase focal adhesion formation in vascular smooth muscle cells, (iv) inhibit dedifferentiation of vascular smooth muscle cells, (v) promote actin polymerization in vascular smooth muscle cells, (vi) increase the ratio of F:G actin in vascular smooth muscle cells, (vii) decrease or inhibit occlusion of a vessel, (viii) decrease or inhibit vascular obstruction, (ix) decrease or inhibit restenosis, and/or (x) prevent stenosis. Mimetics that are either bioactive in the subject methods or function antagonistically may be readily tested using any in vitro method known in the art and outlined in detail herein. Similarly mimetics having the desired activity (e.g., bioactive or antagonistic) can be selected by testing a given mimetic in vivo and assessing whether said mimetic promotes elastin signaling in vascular smooth muscle cells in vivo.

(vi) Method of Screening

This application describes methods and compositions for promoting elastin signaling in smooth muscle cells. With the importance of providing effective methods and compositions for preventing and/or decreasing occlusion of vessels in mind, the present invention contemplates methods of identifying agents which activate and/or promote elastin signaling in smooth muscle cells. Exemplary agents can influence the proliferation, differentiation, and cellular behavior of smooth muscle cells, including vascular smooth muscle cells, when administered in an effective amount.

As described in detail herein, agents which can antagonize the activity of an agent that promotes elastin signaling also have substantial utility. Such antagonists may be used to limit the rate or extent of elastin signaling. Accordingly, the present invention further contemplates methods of identifying agents which antagonize elastin signaling. Exemplary agents can modulate the rate and/or extent of elastin signaling in smooth muscle cells when administered in an effective amount.

Agents screened (e.g., a single agent, a combination of two or more agents, a library of agents) include nucleic acids, peptides, proteins, antibodies, antisense RNAs, RNAi constructs (including siRNAs), chemical compounds, and small organic molecules. Agents may be screened individually, in combination, or as a library of agents.

Without being bound by theory, an agent identified by the subject methods may function in any of a number of way. Exemplary agents include, but are not limited to (a) nucleic acids encoding tropoelastin, (b) nucleic acids encoding a bioactive fragment of tropoelastin, (c) nucleic acids encoding the elastin receptor, (d) nucleic acids encoding a constitutively elastin receptor, (e) nucleic acids encoding Gi heterotrimeric G protein, (f) nucleic acids encoding a constitutively active Gi heterotrimeric G protein, (g) nucleic acids encoding a RhoA GTPase, (h) nucleic acids encoding an constitutively activate RhoA GTPase, (i) polypeptides comprising an amino acid sequence of tropoelastin, (j) polypeptides comprising an amino acid sequence of a bioactive fragment of tropoelastin, (k) polypeptides comprising an amino acid sequence of the elastin receptor, (l) polypeptides comprising an amino acid sequence of a constitutively active elastin receptor, (m) polypeptides comprising an amino acid sequence of a Gi heterotrimeric G protein, (n) polypeptides comprising an amino acid sequence of a constitutively active Gi heterotrimeric G protein, (o) polypeptides comprising an amino acid sequence of a RhoA GTPase, (p) polypeptides comprising an amino acid sequence of a constitutively active RhoA GTPase, (q) polypeptides comprising an amino acid sequences that bind to and activate the elastin receptor in the presence or absence of ligand, (r) small organic molecules that increase the expression and/or activity of Gi, (s) small organic molecules that increase the expression and/or activity of the elastin receptor, (t) small organic molecules that bind to and activate the elastin receptor in the presence or absence of ligand, (u) small organic molecules that bind to tropoelastin and increase the affinity of elastin for the elastin receptor, (v) small organic molecules that bind to the elastin receptor and increase the affinity of elastin for the elastin receptor, (w) small organic molecule that increases the activity and/or expression of RhoA, (x) small organic molecules which increase the “on” state of RhoA, (y) antisense oligonucleotides which decrease the expression of a GAP which inhibits the activity of RhoA, (z) RNAi oligonucleotides or constructs which decrease the expression of a GAP which inhibits the activity of RhoA, (aa) antibodies which bind to and inhibit the activity of a GAP which inhibits the activity of RhoA, (bb) ribozymes which bind to and inhibit the activity of a GAP which inhibits the activity of RhoA, (cc) nucleic acids encoding a GEF which promotes the “on” state of a RhoA, (dd) polypeptides comprising an amino acid sequence of a GEF which promotes the “on” state of a RhoA, (ee) small organic molecules which promote the expression and/or activity of a GEF which promotes the “on” state of RhoA, (ff) small organic molecules which bind to and promote the expression and/or activity of a GEF which promotes the “on” state of RhoA, (gg) antisense oligonucleotides that decrease the expression of a GDI which promotes the “off” state of RhoA, (hh) RNAi constructs that decrease the expression of a GDI which promotes the “off” state of RhoA, (ii) ribozymes that decrease the expression of a GDI which promotes the “off” state of RhoA, (jj) antibodies that decrease the expression and/or activity of a GDI which promotes the “off” state of RhoA, (kk) small organic molecules that decrease the expression and/or activity of a GDI which promotes the “off” state of RhoA, (ll) small organic molecules that bind to and decrease the expression and/or activity of a GDI which promotes the “off” state of RhoA.

The foregoing mechanisms are by no means exhaustive. Agents for use in the subject methods either promote the expression and/or activation of the elastin receptor, promote the expression and/or activation of Gi, promote the expression and/or “on” state of RhoA, increase the expression and/or activity of a protein which promotes the “on” state of RhoA, or decrease the expression and/or activity of a protein which promotes the “off” state of RhoA.

Similarly, and without being bound by theory, exemplary antagonists identified by the subject methods may function in any of a number of ways.

In many drug screening programs that test libraries of nucleic acids, polypeptides, chemical compounds and natural extracts, high throughput assays are desirable to increase the number of agents surveyed in a given period of time. Assays that are performed in cell-free systems, such as may be derived with purified or semi-purified proteins, are often preferred as “primary” screens in that they can be generated to permit rapid development and relatively easy detection of an alteration in a molecular target which is mediated by a test agent. Cell free systems include in vitro systems (preparations of proteins and agents combined in a test tube, Petri dish, etc.), as well as cell free systems such as those prepared from egg extracts or reticulocyte lysates. Moreover, the effects of cellular toxicity and/or bioavailability of the test agents can be generally ignored in such a system, the assay instead being focused primarily on the effect of the agent.

A primary screen can be used to narrow down agents that are more likely to have an affect on elastin signaling in vascular smooth muscle cells, in vitro and/or in vivo. Such a cell free system for use in the present invention may include, for example, a biochemical assay measuring activity of a RhoA GTPase or activity of a heterotrimeric Gi protein. For example, a RhoA GTPase may be contacted with one or more agents (e.g., individual candidate agents, combinations of two or more agents, a library of nucleic acids, polypeptides, small organic molecules, chemical compounds, etc.) and the ability of the agent to promote the “on” state of the RhoA GTPase can be measured. One or more agents that promote the “on” state of a RhoA GTPase are candidate agents for use in the subject methods. Similarly, the application contemplates screening, for example, for agents that promotes elastin signaling by promoting the binding of elastin or an elastin peptide to the elastin receptor, agents that activate, in either the presence or absence of ligand binding, the elastin receptor, agents that activate Gi, and the like.

The efficacy of the agent can be assessed by generating dose response curves from data obtained using various concentrations of the test agent. Moreover, a control assay can also b e performed to provide a baseline for comparison. Such candidates can be further tested for efficacy in promoting elastin signaling in vitro in vascular smooth muscle cells, for efficacy in promoting elastin signaling in vascular smooth muscle cells in vivo, for efficacy in decreasing occlusion of a vessel in vivo, and for efficacy in modulating the cellular and/or morphological properties of a vascular smooth muscle cell.

In the foregoing screening methods, the application further contemplates that screening assays may be performed to identify agents that antagonize elastin signaling. For example, a RhoA GTPase may be contacted with one or more agents (e.g., individual candidate agents, combinations of two or more agents, a library of nucleic acids, polypeptides, small organic molecules, chemical compounds, etc.) and the ability of the agent to promote the “off” state of the RhoA GTPase can be measured. One or more agents which promotes the “off” state of a RhoA GTPase are candidate antagonists for use in inhibiting elastin signaling. Similarly, the application contemplates screening for agents that inhibit the activation of the elastin receptor, agents that inhibit the binding of elastin or an elastin fragment to the elastin receptor, agents that inhibit, in either the presence or absence of ligand binding, the activation of the elastin receptor, agents that inhibit Gi activation, and the like.

In addition to cell-free assays, such as described above, the application further contemplates the generation of cell-based assays for identifying agents that modulate elastin signaling in vascular smooth muscle cells. For example, the application contemplates assays for identifying agents that (increase or decrease) the “on” or “off” state of a RhoA GTPase, agents that (increase or decrease) the activation of Gi, and agents that increase the activation (either in the presence or absence of ligand) of an elastin G-protein coupled receptor. Cell-based assays may be performed as either a primary screen, or as a secondary screen to confirm the activity of agents identified in a cell free screen, as outlined in detail above. Such cell based assays can employ, for example, wild type or mutant vascular smooth muscle cells or smooth muscle cells. Exemplary mutant vascular smooth muscle cells are cells derived from an elastin +/− or elastin −/− animal.

One class of agents that may promote elastin by promoting the activity of a particular protein are agents that bind directly to that particular protein. For example, agents that bind directly to a RhoA GTPase (e.g., promote the “on” state of a RhoA GTPase, inhibit the “off” state of a RhoA GTPase, promote the “off” state of a RhoA GTPase, and/or inhibit the “on” state of a RhoA GTPase). By way of further example, some agents that promote elastin signaling may act by (i) binding directly to elastin or an elastin fragment and enhancing the ability of the protein to bind to and activate the elastin receptor, (ii) binding directly to the elastin receptor to potentiate elastin signaling in either the presence or in the absence of ligand, and (iii) binding directly to and activating Gi. Accordingly, the present invention contemplates screening for agents which bind to, either directly or indirectly (e.g., via a cofactor or accessory protein), a component of the elastin signaling pathway. Many methods exist in the art for assessing protein-protein, protein-nucleic acid, protein-antibody, and protein-small molecule interactions. Exemplary methods include two-hybrid screens, affinity chromatography, immunoprecipitation, and the like. One of skill in the art can select commonly used methods for detecting the interaction of a protein involved in the elastin signaling pathway with an agent including proteins, nucleic acids, small molecule, chemical compounds, antibodies, etc.

(vii) Methods of Administration of Nucleic Acids, Proteins, Chemical Compounds and Pharmaceutical Compositions of Agents

An agent identified by the subject methods has many potential uses. Such an agent may be a nucleic acid, peptide, polypeptide, peptidomimetic, RNAi construct, chemical compound, small organic molecule, antisense RNA, antibody, and the like. By agent is meant to include a single agent, or a combination of agents which together possess the desired activity. Agents that promote elastin signaling in vascular smooth muscle cells may be useful in a therapeutic context to decrease or prevent occlusion of a vessel. Such vessels include vasculature, as well as other vessels selected from common bile duct, pancreatic duct, esophagus, trachea, urethra, bladder, uterus, ovarian duct, Fallopian tube, vas deferens, prostatic duct, tear duct, and lymphatic duct.

Agents that activate elastin signaling for use in the methods of the present invention, as well as agents identified by the subject methods may be conveniently formulated for administration with a biologically acceptable medium, such as water, buffered saline, polyol (for example, glycerol, propylene glycol, liquid polyethylene glycol and the like) or suitable mixtures thereof. Optimal concentrations of the active ingredient(s) in the chosen medium can be determined empirically, according to procedures well known to medicinal chemists. As used herein, “biologically acceptable medium” includes solvents, dispersion media, and the like which may be appropriate for the desired route of administration of the one or more agents. The use of media for pharmaceutically active substances is known in the art. Except insofar as a conventional media or agent is incompatible with the activity of a particular agent or combination of agents, its use in the pharmaceutical preparation of the invention is contemplated. Suitable vehicles and their formulation inclusive of other proteins are described, for example, in the book Remington's Pharmaceutical Sciences (Remington's Pharmaceutical Sciences. Mack Publishing Company, Easton, Pa., USA 1985). These vehicles include injectable “deposit formulations”.

Methods of introduction may also be provided by delivery via a biocompatible, device. Biocompatible devices suitable for delivery of the subject agents include intraluminal devices such as stents, wires, catheters, sheaths, and the like.

The agents identified using the methods of the present invention may be given orally, parenterally, or topically. They are of course given by forms suitable for each administration route. For example, they are administered in tablets or capsule form, by injection, inhalation, ointment, controlled release device or patch, or infusion.

The effective amount or dosage level will depend upon a variety of factors including the activity of the particular one or more agents employed, the route of administration, the time of administration, the rate of excretion of the particular agents being employed, the duration of the treatment, other drugs, compounds and/or materials used in combination with the particular agents employed, the age, sex, weight, condition, general health and prior medical history of the animal, and like factors well known in the medical arts.

The one or more agents can be administered as such or in admixtures with pharmaceutically acceptable and/or sterile carriers and can also be administered in conjunction with other compounds. These additional compounds may be administered sequentially to or simultaneously with the agents for use in the methods of the present invention.

Agents can be administered alone, or can be administered as a pharmaceutical formulation (composition). Said agents may be formulated for administration in any convenient way for use in human or veterinary medicine. In certain embodiments, the agents included in the pharmaceutical preparation may be active themselves, or may be a prodrug, e.g., capable of being converted to an active compound in a physiological setting.

Thus, another aspect of the present invention provides pharmaceutically acceptable compositions comprising an effective amount of one or more agents, formulated together with one or more pharmaceutically acceptable carriers (additives) and/or diluents. As described below, the pharmaceutical compositions of the present invention may be specially formulated for administration in solid or liquid form, including those adapted for the following: (1) delivery via a stent or other biocompatible, intraluminal device; (2) oral administration, for example, drenches (aqueous or non-aqueous solutions or suspensions), tablets, boluses, powders, granules, pastes for application to the tongue; (3) parenteral administration, for example, by subcutaneous, intramuscular or intravenous injection as, for example, a sterile solution or suspension; (4) topical application, for example, as a cream, ointment or spray applied to the skin; or (5) opthalamic administration, for example, for administration following injury or damage to the retina. However, in certain embodiments the subject a gents may be simply dissolved or suspended in sterile water. In certain embodiments, the pharmaceutical preparation is non-pyrogenic, i.e., does not elevate the body temperature of a patient.

Some examples of the pharmaceutically acceptable carrier materials that may be used include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, one or more agents may contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agent of the present invention. These salts can be prepared in situ during the final isolation and purification of the agents of the invention, or by separately reacting a purified agent of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts”, J. Pharm. Sci. 66:1-19)

The pharmaceutically acceptable salts of the agents include the conventional nontoxic salts or quaternary ammonium salts of the agents, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention. These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (See, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the agent which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a agent of the present invention as an active ingredient. An agent of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating a gents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the agents of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active agents, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Transdermal patches have the added advantage of providing controlled delivery of an agent of the present invention to the body. Such dosage forms can be made by dissolving or dispersing the agents in the proper medium. Absorption enhancers can also be used to increase the flux of the agents across the skin. The rate of such flux can be controlled by either providing a rate controlling membrane or dispersing the agent in a polymer matrix or gel.

Ophthalmic formulations, eye ointments, powders, solutions and the like, are also contemplated as being within the scope of this invention.

Pharmaceutical compositions of this invention suitable for parenteral administration comprise one or more agents of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution which, in turn, may depend upon crystal size and crystalline form. Alternatively, delayed absorption of a parenterally administered agent form is accomplished by dissolving or suspending the agent in an oil vehicle.

(viii) Methods of Conducting a Business

The present application further contemplates methods of conducting businesses based on the compositions and methods of the invention. The discovery that agents which activate and/or potentiate elastin signaling can influence the proliferation, differentiation, and cellular characteristics of vascular smooth muscle cells in vitro and in vivo, stimulates the utility of identifying other agents which can activate elastin signaling in smooth muscle cells including vascular smooth muscle cells. Such agents may be useful for influencing elastin signaling in vitro, and additionally may be useful in methods of treating and/or preventing occlusion of vessels in vivo.

In another aspect, the present invention provides a method of conducting a drug discovery business comprising: identifying, by the subject assays, one or more agents which promote elastin signaling in smooth muscle cells; determining if an agent identified in such an assay, or an analog of such an agent, promotes elastin signaling in vivo and/or in vitro and further determining if said agent influences vascular smooth muscle cell behavior; conducting therapeutic profiling of an agent so identified for efficacy and toxicity in one or more animal models; and formulating a pharmaceutical preparation including one or more agents identified as having an acceptable therapeutic profile and which promote elastin signaling thereby influencing vascular smooth muscle cell behavior. Such agents are candidate agents for use in treating and/or preventing occlusion of vessels.

In one embodiment, the drug discovery business further includes the step of establishing a system for distributing the pharmaceutical preparation for sale, and may optionally include establishing a sales group for marketing the pharmaceutical preparation.

In certain embodiments, the initially identified agents can be subjected to further lead optimization, e.g., to further refine the structure of a lead compound so that potency and activity are maintained but balanced with important pharmacological characteristics including:
Solubility
Permeability
Bioavailability
Toxicity
Mutagenicity
Pharmacokinetics—absorption, distribution, metabolism, elimination of the drug
   Structural modifications are made to a lead compound to address issues with the parameters listed above. These modifications however, must take into account possible effects on the molecule's potency and activity. For example, if the solubility of a lead compound is poor, changes can be made to the molecule in an effort to improve solubility; these modifications, however, may negatively affect the molecule's potency and activity. SAR data are then used to determine the effect of the change upon potency and activity. Using an iterative process of structural modifications and SAR data, a balance is created between these pharmacological parameters and the potency and activity of the compound.

Candidate agents, or combinations thereof, must them be tested for efficacy and toxicity in animal models. Such therapeutic profiling is commonly employed in the pharmaceutical arts. Before testing an experimental drug in humans, extensive therapeutic profiling (preclinical testing) must be completed to establish initial parameters for safety and efficacy. Preclinical testing establishes a mechanism of action for the drug, its bioavailability, absorption, distribution, metabolism, and elimination through studies performed in vitro (that is, in test tubes, beakers, petri dishes, etc.) and in animals. Animal studies are used to assess whether the drug will provide the desired results. Varying doses of the experimental drug are administered to test the drug's efficacy, identify harmful side-effects that may occur, and evaluate toxicity.

Briefly, one of skill in the art will recognize that the identification of a candidate agent in a drug based screen is a first step in developing a pharmaceutical preparation useful for promoting elastin signaling in vitro or in vivo. Administration of an amount of said pharmaceutical preparation effective to influence vascular smooth muscle cell behavior must be both safe and effective. Early stage drug trials, routinely used in the art, help to address concerns of the safety and efficacy of a potential pharmaceutical. In the specific case of an agent that promotes elastin signaling in vascular smooth muscle cells, efficacy of the pharmaceutical preparation could be readily evaluated in normal or mutant vascular smooth muscle cell lines, or in vivo or in vitro in a mouse model. Briefly, mice could be administered varying doses of said pharmaceutical preparations over various time schedules. The route of administration would be appropriately selected based on the particular characteristics of the agent. Control mice can be administered a placebo (e.g., carrier or excipient alone).

In one embodiment, the step of therapeutic profiling includes toxicity testing of compounds in cell cultures and in animals; analysis of pharmacokinetics and metabolism of the candidate drug; and determination of efficacy in animal models of diseases. In certain instances, the method can include analyzing structure-activity relationship and optimizing lead structures based on efficacy, safety and pharmacokinetic profiles. The goal of such steps is the selection of drug candidates for pre-clinical studies to lead to filing of Investigational New Drug applications (“IND”) with the FDA prior to human clinical trials.

Between lead optimization and therapeutic profiling, one goal of the subject method is to develop an agent which has minimal side-effects. In the case of agents for in vitro use, the lead compounds should not be exceptionally toxic to cells in culture, should not be mutagenic to cells in culture, and should not be carcinogenic to cells in culture. In the case of agents for in vivo use, lead compounds should not be exceptionally toxic (e.g., should have only tolerable side-effects when administered to patients), should not be mutagenic, and should not be carcinogenic.

By toxicity profiling is meant the evaluation of potentially harmful side-effects which may occur when an effective amount of a pharmaceutical preparation is administered. A side-effect may or may not be harmful, and the determination of whether a side effect associated with a pharmaceutical preparation is an acceptable side effect is made by the Food and Drug Administration during the regulatory approval process. This determination does not follow hard and fast rules, and that which is considered an acceptable side effect varies due to factors including: (a) the severity of the condition being treated, and (b) the availability of other treatments and the side-effects currently associated with these available treatments. For example, the term cancer encompasses a complex family of disease states related to mis-regulated cell growth, proliferation, and differentiation. Many forms of cancer are particularly devastating diseases which cause severe pain, loss of function of the effected tissue, and death. Chemotheraputic drugs are an important part of the standard therapy for many forms of cancer. Although chemotherapeutics themselves can have serious side-effects including hair-loss, severe nausea, weight-loss, and sterility, such side-effects are considered acceptable given the severity of the disease they aim to treat.

In the context of the present invention, whether a side-effect is considered significant will depend on the condition to be treated and the availability of other methods to treat that condition. For example, the elastin promoting agent may be used to prevent restenosis following angioplasty or may be used to prevent or decrease occlusion of a vessel such as a vas deferens or Fallopian tube. One of skill in the art must assess whether any side-effect is acceptable based on the overall health of the individual, the availability of other treatments, and whether the condition being treated is life-threatening or otherwise significantly impacts the quality of life of the sufferer.

Toxicity tests can be conducted in tandem with efficacy tests, and mice administered effective doses of the pharmaceutical preparation can be monitored for adverse reactions to the preparation.

An agent or agents which promote elastin signaling in smooth muscle cells, and which are proven safe and effective in animal studies, can be formulated into a pharmaceutical preparation. Such pharmaceutical preparation can then be marketed, distributed, and sold. Sale of these agents may either be alone, or as part of a therapeutic regimen including evaluation by a physician, appropriate treatment, and appropriate after-care in coordination with the treating physician or with another licensed physician or health care provider.

### EXEMPLIFICATION

The invention now being generally described, it will be more readily understood by reference to the following examples which are included merely for purposes of illustration of certain aspects and embodiments of the present invention, and are not intended to limit the invention.

### Example 1

### Elastin Regulates the Contractile Phenotype of Vascular Smooth Muscle Cells

Tropoelastin, the gene product of the elastin gene, is the major extracellular matrix protein in an artery and is synthesized and deposited by vascular smooth muscle cells (vsmcs). Mature arterial smooth muscle cells exist in a quiescent state specialized for contraction, with highly organized actin stress fibers. In response to vascular insults, these cells change their phenotype, lose their actin stress fibers, proliferate and migrate into the arterial lumen. Elastin signaling regulates the phenotypic modulation of vascular smooth muscle cells from a quiescent contractile state to a proliferative non-contractile state. As demonstrated herein, the elastin receptor is not a member of the integrin family of matrix receptors (divalent cation chelator insensitive) and is a G protein coupled receptor (pertussis toxin sensitive) that signals through both Gi and Gs families of heterotrimeric receptors.

Elastin is a critical autocrine regulator of vascular smooth muscle cell development and biology. Disruption of the elastin matrix caused either by inflammation, direct mechanical injury, and/or genetic defect is central to the pathogenesis of obstructive vascular diseases. In the absence of elastin or proper signaling via elastin, vascular smooth muscle cells dedifferentiate, proliferate and migrate to the lumen occluding arteries. Restoring appropriate elastin expression or elastin signaling to the vascular wall will prevent obstructive vascular diseases regardless of whether the particular disease is caused by genetic defect, injury, or inflammation. The invention contemplates that methods of delivering elastin or other agents which potentiate elastin signaling can be via a stent, sheath, wire, or other intraluminal device which allows delivery to or near the site of vascular obstruction. The invention further contemplates systemic delivery of elastin and/or agents which potentiate elastin signaling, as well as local delivery to or near a site of vascular obstruction without the use of an intraluminal device.

Elastin regulates contractile phenotype. We isolated vascular smooth muscle cells from Eln +/+ and Eln −/− aortic explants. We confirmed that these cells express the vsmc markers desmin, SM-22, calponin and alpha smooth muscle actin (α-SM) using reverse transcriptase/polymerase chain reaction (RT/PCR) analysis. Using RT/PCR, in situ hybridization, and immunofluorescence, we demonstrated that early passage (passage 4-5) Eln +/+ vsmcs express and synthesize elastin, while Eln −/− vsmcs did not express or synthesize elastin.

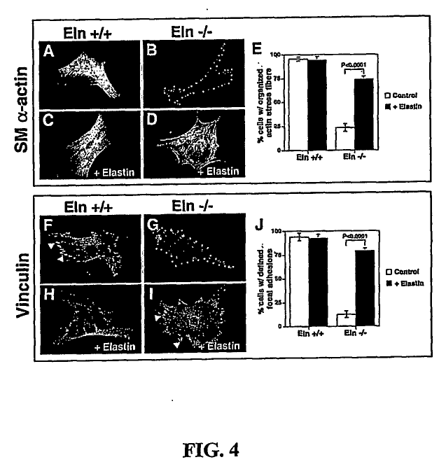

Arterial smooth muscle cells normally exist in a quiescent state specialized for contraction with highly organized actin stress fibers. Following vascular injury, however, these cells dedifferentiate from a contractile to a synthetic state, characterized by the loss of the contractile apparatus and dramatic increases in the rates of cell migration and proliferation. The hallmark of a contractile state is highly organized actin myofilaments, often referred to as actin stress fibers. We isolated vascular smooth muscle cells from Eln −/− mice and Eln +/+ m ice. Well-defined actin stress fibers are apparent in more than 95% of Eln +/+ cells scored by observers blinded to genotype (FIGS. 4A and 4E). In contrast, only 23% of Eln −/− cells have actin stress fibers (FIGS. 4B and 4E). Within 3 hours of exposure to 1.0 ug/ml recombinant tropoelastin, the percentage of Eln −/− cells with organized actin stress fibers increased 3-fold to 74% (FIG. 4D to 4E). In comparison, elastin treatment had no affect on actin stress fiber organization in Eln +/+ cells (FIGS. 4C and 4E). Similar results are observed when the organization of vinculin, a focal adhesion protein, is examined by immunohistochemistry (FIG. 4F to 4J). Elastin's activity was independent of whether serum was present. Thus, elastin directly induces the organization of contractile apparatus in vascular smooth muscle cells.

### Example 2

### Tropoelastin Modulates Migration of Vascular Smooth Muscle Cells

In Eln −/− arteries, vascular smooth muscle cells accumulated in the subendothelial space and occluded the artery. These data suggested that the elastic lamellae localize vsmcs and regulate their proliferation. Because the elastic lamellae are highly fenestrated allowing cells to migrate through, it is unlikely that elastin simply forms a physical cage that contains vsmcs. Instead, we postulated that elastin signals vsmcs to migrate and adhere to the elastic lamellae. To test this hypothesis we used a modified Boyden chamber assay (Li et al., 1998; Raines et al., 1993). This assay system consists of two adjacent chambers separated by a semipermeable membrane. The amount of cells placed in one chamber (upper) that migrate to the other chamber (lower) is measured by counting the number of cells that have passed through to the undersurface of the filter.

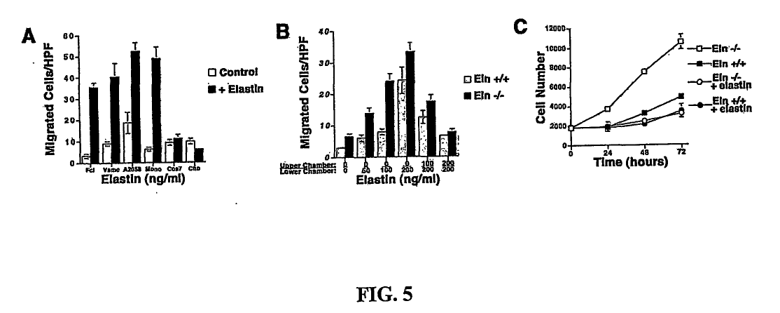

Our migration data showed three important results. First, murine vsmcs migrated aggressively to recombinant tropoelastin (FIG. 5A). No migration was observed when COS, and CHO cells were exposed to elastin. The amount of migration induced by recombinant tropoelastin is equivalent to the standard dose of 30 ng/ml of platelet derived growth factor (data not shown). These data indicated that migration to tropoelastin is cell-specific. Second, we carried out a checkerboard analysis to demonstrate that the migration of vsmcs was dependent on the concentration gradient of tropoelastin, not the total amount used in the assay (FIG. 5B). For example, there was minimal migration with a 0 ng to 0 ng gradient or a 200 ng to 200 ng gradient Thus, tropoelastin is a chemotactic agent and not simply a chemokinetic a gent. Third, Eln +/+ vsmcs did not migrate towards exogenous tropoelastin as readily as Eln −/− vsmcs (FIG. 5B). Eln +/+ vsmcs synthesized and secreted tropoelastin, lessening the gradient between chambers. In this model, destruction of the elastin matrix, whether due to injury, disease, or genetic defect in the elastin gene, would allow other stimuli such as growth factors and chemokines to attract vsmcs away from its organized medial ring leading to aberrant accumulation in an artery's lumen.

FIG. 5C further illustrates changes in cell proliferation in vascular smooth muscle cells with defects in elastin signaling. Eln −/− vsmcs proliferated faster than Eln +/+ vsmcs. With the addition of recombinant tropoelastin to the media, the proliferation of Eln −/− and Eln +/+ vsmcs was reduced to similar levels. All of the proliferation, migration, and differentiation data presented argues that elastin signaling induces arterial stabilization and maturation.

### Example 3

### Elastin Signals Through a G-Protein Coupled Receptor

G-protein coupled receptors (GPCRs) are the largest family of transmembrane receptors that are activated by ligands including amino acids, peptides, nucleotides, lipids, retinal and phermones. Examples of such receptors include the adrenergic receptors. Ligands binding to GPCRs cause a conformational change and activate intracellular heterotrimeric G proteins (α, β and γ subunits). The effector G proteins are classified into four protein families based on composition of the α-subunit: Gₛ, Gᵢ, G_{q/11}, and G_{olf}. Each class of effector G-proteins activates distinct downstream biochemical pathways. The availability of unique inhibitors and biochemical markers specific for each class of G-proteins allows the dissection of the molecular nature of signaling in response to a particular ligand. All GPCRs regardless of which G protein they activate share a common structure of seven transmembrane domains.

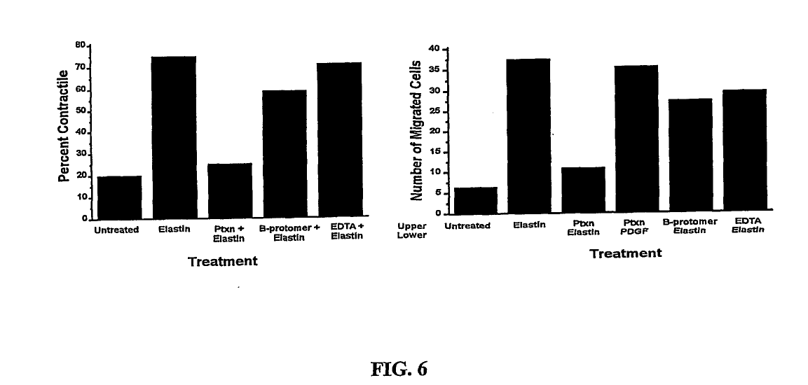

In order to dissect the pathway through which elastin signals, we assessed the effect of various G protein inhibitors on vascular smooth muscle cells. FIG. 6 summarizes the results of these experiments. In the presence of pertussis toxin (100 ng/ml), a specific Gᵢ inhibitor, elastin fails to induce migration or a contractile phenotype in vascular smooth muscle cells. These results support a model whereby the elastin receptor is a GPCR expressed in vascular smooth muscle cells that interacts with Gᵢ. However, a caveat when interpreting all inhibitor experiments is that the phenotype observed can be non-specific. A control experiment to demonstrate the specificity of the result obtained following exposure of vsmcs to pertussis toxin is based on the following. Pertussis toxin is composed of two protomers, A and B. The A protomer encodes the portion of the toxin that poisons Gi while the B protomer binds to cell surface lectins enabling the toxin to enter the cell. If the above described effect on vsmcs is specific, then one would not expect the expression of the B-protomer alone (in the absence of the portion of the toxin responsible for Gi inhibition) to disrupt elastin induced contractile organization or migration. Our results are consistent with this prediction (FIG. 6). Thus disrupted G protein signaling causes the inhibition of migration and contractile organization by pertussis toxin (A and B protomer), and the specificity of this result supports the hypothesis that the elastin receptor is a GPCR that activates Gi.

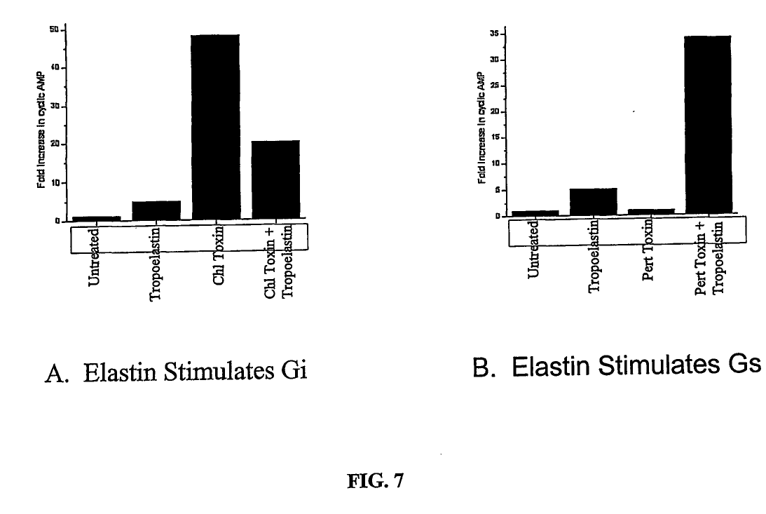

FIG. 7 summarizes a second set of experiments that demonstrated that elastin signaling modulates the level of cAMP. Cholera toxin activates Gs and results in adenylyl cyclase stimulation and elevation of cAMP. Treatment of vascular smooth muscle cells with cholera toxin leads to increased cAMP levels (FIG. 7A). When treated with both cholera toxin and elastin, the levels of cAMP are significantly reduced. Thus elastin activates Gi which inhibits adenylyl cyclase activity. This data is consistent with our previous observation that elastin induced cellular activity was sensitive to pertussis toxin, the specific Gi inhibitor. Interestingly, there is also evidence for a second G-protein pathway (FIG. 7B). In the presence of pertussis toxin, elastin raises the level of cAMP indicating that adenylyl cyclase is activated. This data suggests that in addition to Gi, elastin also activates Gs, the G-protein that stimulates adenylyl cyclase. These data indicate that similar to other GPCRs, the elastin receptor is a GPCR that can signal through two distinct heterotrimeric G-proteins.

### Example 4

### Elastin Signaling Activates a Specific Potassium Channel

Further experiments indicate that elastin signaling has specific effects on cell physiology, and that these effects are mediated by a G-protein coupled receptor. Our preliminary data indicated that one of the biologically significant results of elastin activation of Gi is the activation of a Gi specific potassium channel. Not only does Gi inhibit adenyl cyclase, as described in detail above, but it also signals by stimulating GIRK (G-protein gated inward rectifier K+ channel) potassium channels. Functional GiRK potassium channels are heteromultimers of GiRK 1 and GiRK 4 (Logothetis et al. (1987) Nature 325: 321-326; Krapivinsky et al. (1995) Nature 374: 135-141). Our experiments, performed in collaboration with David Clapham, indicated that activation of GIRK channels is an immediate response to activation of elastin signaling via treatment with tropoelastin.

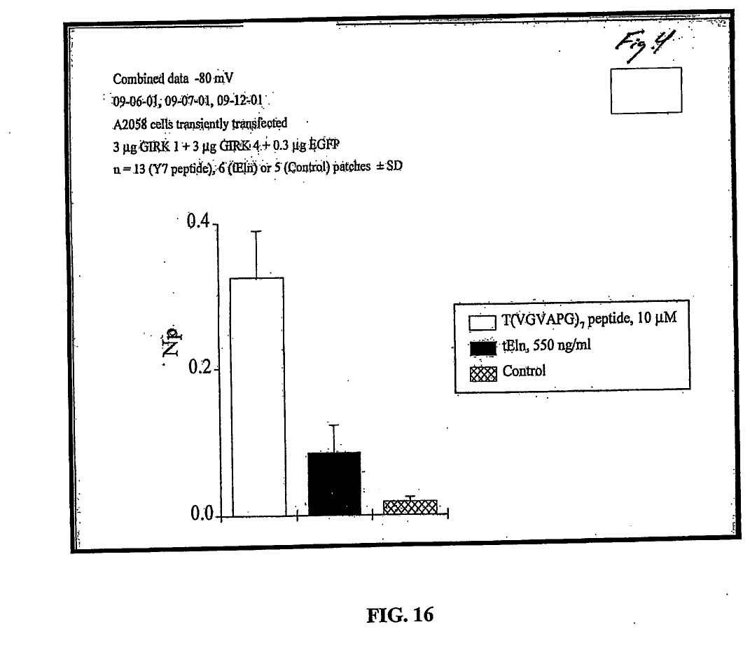

FIG. 16 summarizes some additional experiments demonstrating that GiRK potassium channels are activated in response to elastin signaling. Briefly, A2058 cells which are known to be responsive to elastin signaling were used in these studies. A2058 cells were transiently transfected with GiRK 1 and GIRK 4 inorder to form functional heteromultimeric GiRK channels. Transfected cells were contacted with either tropoelastin, a peptide containing 7 repeats of the tropoelastin fragment VGVAPG, or a control peptide. Contacting cells with either the tropoelastin peptide or tropoelastin, but not a control peptide, resulted in activation of GiRK channel activity.

These experiments not only help to further clarify the elastin signaling pathway, but also provide another functional response to elastin signaling which can be used to monitor the response of cells to agents which activate elastin signaling. Based on these results, one would expect that activation of GiRK channel activity can be used as a marker for assessing activation of elastin signaling.

### Example 5

### Elastin Signaling Modulates Vascular Smooth Muscle Cells In Vivo

The disruption of elastin signaling, in vivo, contributes to the pathogenesis of obstructive vascular diseases. Support for this model comes from four lines of evidence: pathological, genetic, experimental, and therapeutic. Disruption of the elastin matrix is consistently associated with obstructive vascular diseases in human pathologic specimens (Hinek et al., 1996; Yow et al., 1988; Hinek et al., 1993). Moreover, the severity of obstructive vascular pathology increases in proportion to the magnitude of defects and discontinuities in the elastin matrix (Privitera et al., 1998). We previously showed that loss-of-function mutations in one elastin allele cause a particular human obstructive vascular disease, supravalvular aortic stenosis (Curran et al., 1993; Ewart et al., 1993). In gene-targeting experiments, we demonstrated that loss of elastin in mice is sufficient to cause obstructive vascular pathology (Li et al., 1997). We further demonstrated that this pathology was caused by the unregulated proliferation, migration and accumulation of vascular smooth muscle cells in the subendothelial space. We now provide evidence to support the use of agents which modulate elastin signaling in vivo to decrease vasculature obstructions.

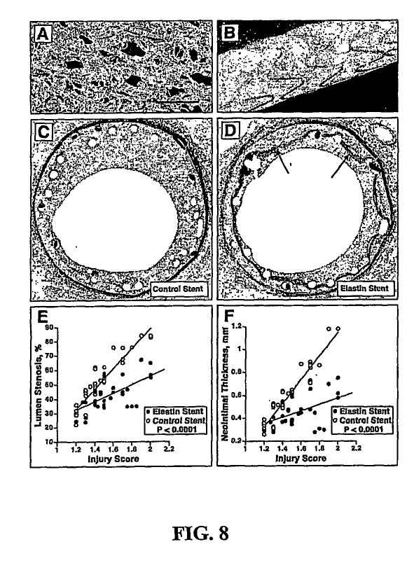

To test the therapeutic potential of elastin biomaterials, we generated sheaths of elastin matrix from porcine carotid arteries using a modified detergent extraction protocol (Mecham et al., 1991; Grosso et al., 1991). Amino acid composition and desmosine analysis indicated that there were no contaminants in the elastin sheaths (Casterella and Tierstein, 1997). The sheaths were wrapped around coronary stents to form elastin sheath-stents, and placed in a porcine coronary artery (FIG. 8A, 8B). After three days, no evidence of acute inflammation or thrombosis was observed. These experiments demonstrated that elastin biomaterials could be delivered to the site of vascular injury effectively.

Vascular injury caused by the placement of coronary stents stimulates vsmc proliferation and accumulation that occludes the lumen of arteries. We postulated that delivery of elastin to the site of injury caused by the stent would reduce this subendothelial accumulation of vsmcs or neointima. To determine if the stimulation of elastin signaling by the on-stent delivery of elastin reduced neointima accumulation, we used an established porcine model of restenosis (Rezai et al., 2001; Leon et al., 2001). The left and right coronary arteries of domestic pigs received either elastin sheath-stents or identical control stents without the elastin sheaths. Four weeks after injury and stent placement, we measured the neointimal thickness (NIT), neointimal area, lumen area, percent stenosis, and mean injury score in each artery by quantitative morphometry. Briefly, injury caused by the contact between the metallic surface or strut of a stent against the vessel wall was scored on a graded scale of 0-3. A score of 1 indicated disruption of the internal elastic lamella; a score of 2 indicated laceration of the media; and a score of 3 indicated damage to the external elastic lamella. The accepted standard for measuring mean injury score for each section involves adding up the injury caused by each strut and dividing by the number of struts. Injury measurements are required in restenosis studies because the degree of restenosis is directly proportional to the amount of injury inflicted by stent placement (Sousa et al., 2001). Without these measurements, any perceived improvement caused by an experimental treatment may simply be due to a reduced mean injury score in the experimental group in comparison to the control group. Thus, valid interpretation of data from animal models requires that the degree of restenosis in experimental and control stents must be measured over a range of comparable injury scores.

Stimulation of elastin signaling using elastin delivered on a sheath reduced coronary restenosis (FIG. 8C-8D). Histology sections taken from the control arteries displayed the development of thick cellular neointima. Immunostains for α-SM actin confirmed that the neointima was composed of >95% smooth muscle cells (data not shown). Van Gieson staining of arteries treated with elastin sheath-stents for 28 days confirmed the presence and stability of the elastin sheath. There was a significant reduction in all restenosis measurements, including the NIT and percent stenosis observed in the elastin treated arteries across the entire range of mean injury scores (P<0.001). Thus, the stimulation of elastin signaling via delivery of elastin to the site of vascular injury reduced the proliferation and accumulation of vsmcs.

### Example 6

### Characterization of Vascular Smooth Muscle Cells Derived from Elastin −/− Mice

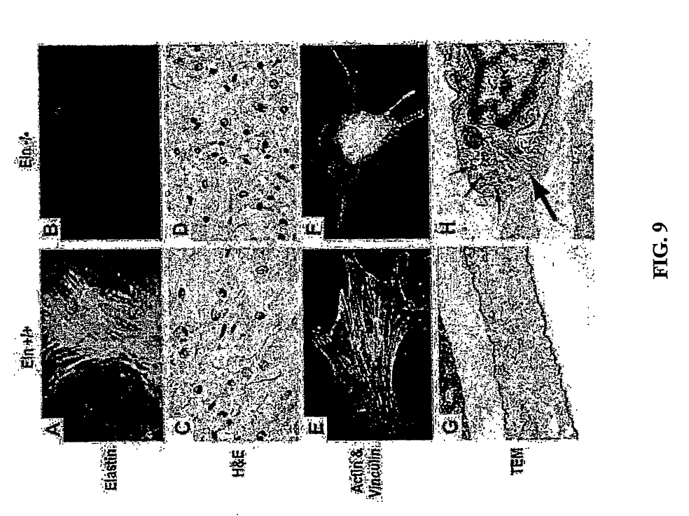

FIG. 9 details experiments designed to further characterize vascular smooth muscle cells derived from elastin −/− mice in comparison to vascular smooth muscle cells derived from elastin +/+ mice. To establish cultures of vascular smooth muscle cells, newborn elastin +/+ and −/− mice (postnatal day 0.5) were sacrificed using CO₂. Following dissection of the ascending aortas, the medial layer was cultured in individual wells of a 12-well culture dish containing Amniomax C-100 growth medium (Gibco-BRL) supplemented with penicillin/streptomycin. Cells were cultured at 37° C. in 5% CO₂. Cultures of smooth muscle cells formed around the tissue, and these cells were trypsinized, passaged, and expanded. Expanded putative smooth muscle cells were genotyped to confirmed their identity, and assayed both morphologically and using molecular markers to confirm that the expanded cells were vascular smooth muscle cells. Molecular markers used to confirm the identity of the cells included: smooth muscle α-actin, smooth muscle γ-actin, SM-22, calponin, desmin, Ang-1/Ang-2. Additionally, the desired cells were assessed to confirm that they did not express endothelial markers including Flk-1 and Flt-1.

FIG. 9 summarizes the results of experiments designed to characterize vascular smooth muscle cells derived from both elastin +/+ and elastin −/− mice. Briefly, FIGS. 9A and 9B show that cells derived from elastin +/+ mice express tropoelastin, as assayed by immunohistochemistry using an anti-tropoelastin antibody, whereas vascular smooth muscle cells derived from elastin −/− mice do not express tropoelastin protein.

FIGS. 9C and 9D provide H&E staining which revealed substantial morphological differences between cells derived from +/+ and −/− animals. H &E staining was performed according to the manufacturers instruction using a Hema 3 stain kit (Fisher). Cells derived from elastin +/+ mice have an elongated and spindle shaped-morphology which is consistent with a contractile phenotype. Cells derived from elastin −/− mice have a rounder, epitheliod morphology consistent with a non-contractile phenotype. Changes in cell morphology were further assessed by examining +/+ and −/− cells using transmission electron microscopy (TEM) (FIGS. 9G and 9H). TEM was performed according to standard protocol. Confluent cultures of +/+ or −/− vascular smooth muscle cells were fixed in 2.5% glutaraldehyde/1% paraformaldehyde for 1 hour at room temperature. Cells were washed 3 times for 10 minutes in cacodylate buffer. Cells were then stained with 2% aqueous uranyl acetate for one hour and washed with cacodylate buffer 3 times for 10 minutes. Following staining, cells were dehydrated in a graded ethanol series of 70%, 80%, 90% and 100% ethanol for 10 minutes each, infiltrated with 100% epoxy resin, embedded in epoxy resin at 65° C. for 24 hours, and cut into 60-80 nM sections on an LKB Nova Ultramicrotome. Sections were stained in aqueous uranlyl acetate and examined on a Hitachi H-7100 transmission electron microscope. We note that both Golgi apparati (indicated with a large arrow) and rough endoplasmic reticula (indicated with a small arrow) are less abundant in elastin +/+ cells.

In addition to gene expression and cell morphology, we examined the presence of focal adhesions and actin stress fibers in +/+ and −/− cells (FIGS. 9E and 9F). Focal adhesions were assayed using an antibody against vinculin and actin stress fibers were assayed using rhodamine phalloidin. Cells derived from elastin +/+ mice are characterized by the presence of both actin stress fibers and focal adhesions, whereas cells derived from elastin −/− mice are characterized by a reduction or absence of both actin stress fibers and focal adhesions.

### Example 7

### Activation of Elastin Signaling Rescues Actin Organization

As summarized in Example 6, vascular smooth muscle cells derived from elastin −/− mice are characterized by defects in actin polymerization and cytoskeletal organization. To determine whether activation of elastin signaling could rescue these deficits, we treated cultures of elastin −/− vascular smooth muscle cells with either tropoelastin (SEQ ID NO: 2), an elastin peptide (SEQ ID NO: 3), or a control peptide (SEQ ID NO: 4). Briefly, cells were seeded at a density of approximately 1000 cells per well in a 24-well plate and treated for 3 hours with vehicle (untreated), 1 ug/ml tropoelastin (SEQ ID NO: 2), 10 nM elastin peptide (SEQ ID NO: 3), or 10 nM control peptide (SEQ ID NO: 4). Following treatment, cells were fixed for 10 minutes in Zamboni's fixative, washed in Dulbecco's phosphate buffered saline (DPBS, Gibco-BRL), and permeabilized in 0.2% Triton X-100 for 7 minutes. Cells were washed again in DPBS and blocked in 5% skim milk for 20 minutes at room temperature. These cells were subjected to triple-staining with 1:50 Rhodamine-Phalloidin (used to detect actin stress fibers; available from Molecular Probes), 1:200 anti-vinculin antibody (hVin-1, Sigma), and DAPI (used to stain nuclei; available from Molecular Probes).

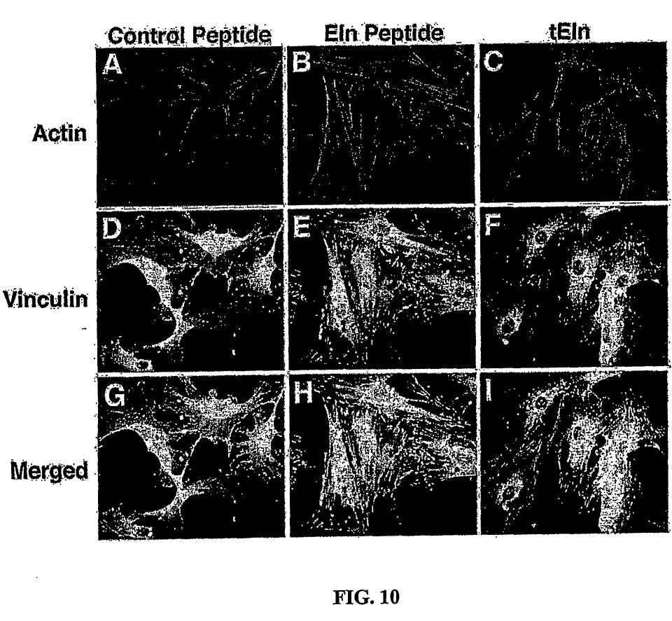

FIG. 10 summarizes the results of these experiments which demonstrated that stimulation of elastin signaling using either tropoelastin or an elastin peptide induced actin polymerization in elastin −/− vascular smooth muscle cells. FIG. 10A-10C shows immunofluorescence using rhodamine-conjugated phalloidin and demonstrated that treatment of the cells with either the elastin peptide (SEQ ID NO: 3) or tropoelastin (SEQ ID NO: 2) induced actin myofilament polymerization. We note that treatment of the cells with a control peptide (SEQ ID NO: 4) did not produce this effect and these cells exhibited only background staining of unpolymerized (globular) actin. FIG. 10D-10F shows immunofluorescence using an antibody which recognizes vinculin and demonstrated that treatment of −/− cells with either the elastin peptide (SEQ ID NO: 3) or tropoelastin (SEQ ID NO:2), but not with the control peptide (SEQ ID NO: 4), induced organization of focal adhesions. FIG. 10G-10I p resents merged images of actin, v inculin, and DAPI staining to confirm that approximately equivalent numbers of cells are presented in each image.

### Example 8

### Activation of Elastin Signaling Rescues Actin Organization in a Dose-Dependent Manner

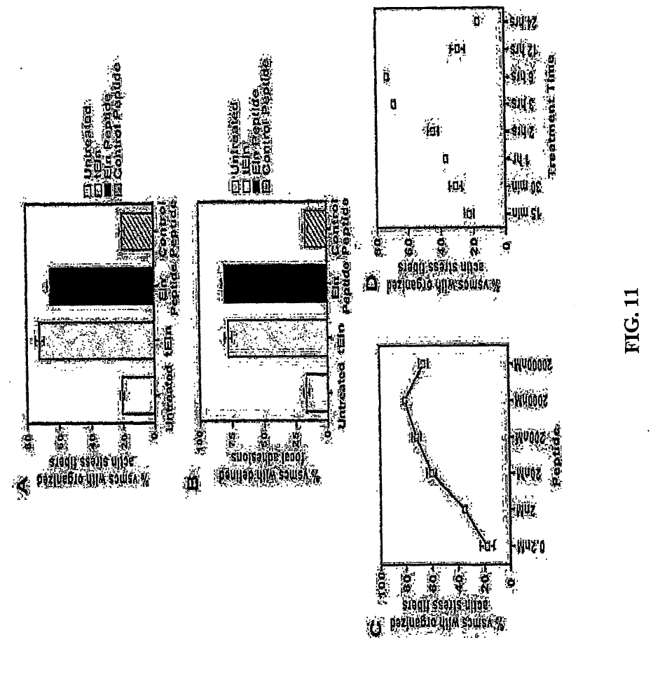

To further demonstrate the validity and specificity of the effect of elastin signaling on actin organization, we attempted to quantify the results presented in FIG. 10 and also analyzed the phenotype of −/− cells treated with a range of concentrations of the elastin peptide. FIGS. 11A and 11B provide graphs summarizing experiments analyzing actin stress fibers and focal adhesions in elastin −/− cells. Briefly, the percentage of −/− vascular smooth muscle cells with organized actin stress fibers and focal adhesions was scored either in the absence of treatment, or following treatment with tropoelastin (SEQ ID NO: 2), an elastin peptide (SEQ ID NO: 3), or a control peptide (SEQ ID NO: 4). Cells were scored as contractile if they exhibited distinct actin stress fibers continuous throughout the cytoplasm, or contained at least 10 well defined focal adhesions distributed throughout the cytoplasm and cell periphery. At least 100 cells were scored in 3 separate cultures, and identical results were obtained whether cells were cultured on glass slides or Matrigel coated plastic tissue culture plates. Scoring analysis confirms a significant increase in the percentage of cells with actin stress fibers and defined focal adhesions following treatment with either tropoelastin or with an elastin peptide (SEQ ID NO: 3) in comparison to either untreated cells or cells treated with control peptide (SEQ ID NO: 4).

Having confirmed and quantified that potentiation of elastin signaling increases both actin stress fiber formation and focal adhesion formation, we examined the dose dependence of administration of the elastin peptide. −/− vascular smooth muscle cells were treated for three hours in concentrations of elastin peptide (SEQ ID NO: 3) ranging from 0.2 nM to 20 μM. FIG. 11C summarizes the results of these experiments which demonstrated that the increase of actin stress fibers in −/− vascular smooth muscle cells following treatment with an elastin peptide is dose dependent.

Finally, we performed a time-course experiment to examine the effect of elastin peptide on actin stress fiber formation following treatments of 15 minutes to 24 hours. The results of this experiment are summarized graphically in FIG. 11D and demonstrated that stimulation of actin stress fiber formation by an elastin peptide is temporally sensitive. The effects occur after approximately one hour of treatment with an elastin peptide, and the maximal effect is observed after about 6 hours of treatment.

These results indicated that elastin induced actin stress fiber formation is a specific, dose-dependent, and saturable event suggesting that elastin (or an elastin peptide) activates a specific receptor to potentiate elastin signaling. Additional experiments will attempt to further elucidate components of the elastin signaling pathway downstream of receptor binding.

### Example 9

### Activation of Elastin Induces Actin Polymerization Via a Post-Translation Mechanism

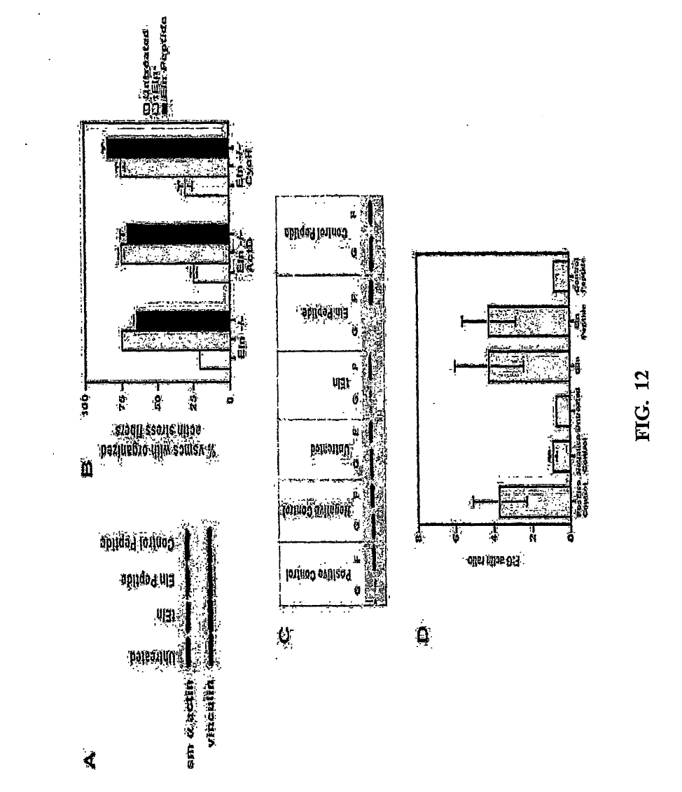

As summarized in FIG. 11D, stimulation of elastin signaling induces actin stress fiber formation in vascular smooth muscle cells relatively rapidly, and the rate at which this occurs suggests a post-translational mechanism. We performed two experiments to address this model. In the first experiment, elastin −/− vascular smooth muscle cells were grown to approximately 70% confluence in T75 flasks (Corning), and either left untreated or treated for three hours with 1 μg/ml tropoelastin, 10 μM elastin peptide (SEQ ID NO: 3), or 10 μM control peptide (SEQ ID NO: 4). Cells were washed with ice-cold DPBS and lysed in an additional 1 ml of Laemmli sample buffer. Following lysis, cells were collected, boiled, pelleted, and resolved by SDS-PAGE. The resolved samples were transferred to a membrane, blocked with 5% milk/PBST, incubated with either an anti-α smooth muscle actin antibody or an anti-vinculin antibody, and visualized using chemiluminescence. FIG. 12A shows the results of this experiment which demonstrated that treatment of vascular smooth muscle cells with either tropoelastin (SEQ ID NO: 2) or with an elastin peptide (SEQ ID NO: 3) does not alter the protein levels of either a smooth muscle actin or vinculin.

In the second experiment, the results of which are summarized in FIG. 12B, we examined the effect of tropoelastin or elastin peptide on actin stress fiber formation in the presence of either actinomycin D (an inhibitor of transcription) or cyclohexamide (an inhibitor of translation). Briefly, as previously described, treatment of −/− vascular smooth muscle cells with either tropoelastin (SEQ ID NO: 2) or an elastin peptide (SEQ ID NO: 3) increased actin stress fiber formation. This effect of tropoelastin or elastin peptide was maintained when cells were pretreated with either 100 μM actinomycin D (Calbiochem) or 10 μg/ml cyclohexamide (Calbiochem) (FIG. 12B). These results indicated that elastin signaling induces actin stress fiber formation in vascular smooth muscle cells via a mechanism that does not require new gene transcription or translation.

### Example 10

### Activation of Elastin Signaling Induces a Change in the Ratio of F:G Actin

The results summarized in FIGS. 12A and 12B suggest that elastin signaling induces actin stress fiber formation via a mechanism that requires neither translation nor new gene transcription. One possible mechanism that would account for actin stress fiber formation independent of new transcription or translation is the post-translational conversion of globular actin (G-actin; the unpolymerized form) to filamentous actin (F-actin; the polymerized form).

In order to test this hypothesis, we assayed F-actin and G-actin in −/− vascular smooth muscle cells that were either untreated or treated with tropoelastin (SEQ ID NO: 2), elastin peptide (SEQ ID NO: 3) or control peptide (SEQ ID NO: 4). The ratio of F:G actin was determined using a commercial F:G actin kit (Cytoskeleton Inc.). Briefly, we isolated protein extracts from treated −/− cells in culture and separated F and G actin using a sedimentation assay. Treatment with phalloidin, a stabilizer of F-actin, served as a positive control, and treatment with cytochalasin D, which disrupts F-actin, served as a negative control. FIGS. 12C and 12D summarizes the results of these experiments demonstrating a 3-fold increase in the F:G actin ratio in cells treated with either tropoelastin (SEQ ID NO: 2) or elastin peptide (SEQ ID NO: 3), but not in untreated cells or cells treated with control peptide (SEQ ID NO: 4).

### Example 11

### Elastin Signaling Mediates Actin Polymerization Via RhoA

Our previous results indicate that elastin signals via activation of a G-protein coupled receptor, and we have provided evidence which suggests that elastin acts via a Gi subunit. Work performed by other investigators has implicated RhoA GTPases in cellular contraction and migration, however, previous studies have not linked RhoA GTPase dependent signaling and Gi subunit dependent signaling. Nevertheless, and given the obvious involvement of elastin signaling in cellular contraction and migration, we asked whether RhoA could mediate elastin signaling dependent actin stress fiber formation.

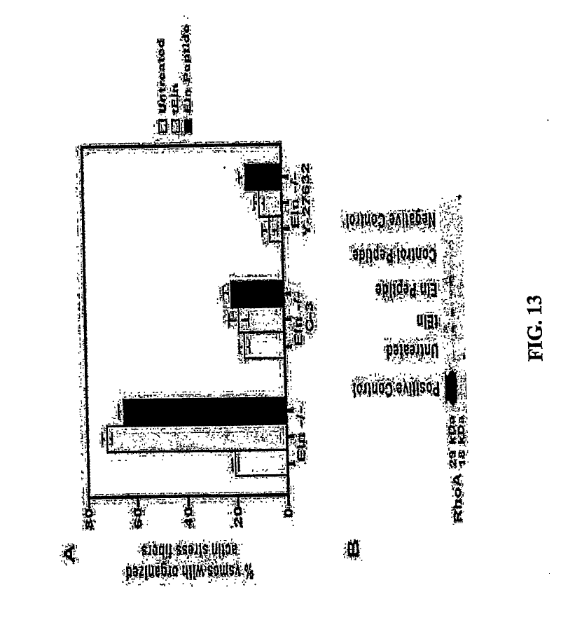

FIG. 13 summarizes a first series of experiments which support a model wherein elastin signaling mediates actin polymerization via the small GTPase, RhoA. Briefly, FIG. 13A shows that following 3 hours of treatment with either tropoelastin (SEQ ID NO: 2) or elastin peptide (SEQ ID NO: 3), 70-80% of elastin −/− vascular smooth muscle cells exhibited actin stress fibers. However, this effect was significantly decreased if cells were pretreated with either C-3 coenzyme or Y-27632. C-3 coenzyme is a Rho GTPase inhibitor and Y-27632 is a Rho kinase inhibitor.

FIG. 13B shows that treatment with elastin peptide or tropoelastin activates RhoA by facilitating the exchange of GDP for GTP. Briefly, cell lysates from treated or untreated −/− cells were immunoprecipitated using Rhotekin-coated beads which recognize GTP-bound RhoA. Western blot anaysis of these lysates using an antibody against Rho demonstrated that both tropoelastin and elastin peptide result in a significant increase of GTP-bound (activated) RhoA. Rho-γGTP served as a positive control and Rho-GDP served as a negative control.

### Example 12

### Elastin Signaling Through Heterotrimeric Gi Proteins Mediates Actin Polymerization

Previous studies implied that the elastin receptor was a G-protein coupled receptor which signaled through heterotrimeric Gi proteins. The results summarized herein which demonstrate that elastin-signaling dependent actin stress fiber formation is mediated by activation of RhoA, an intracellular effector, further supports the existence of an elastin receptor responsible for transducing an extracellular elastin signal.

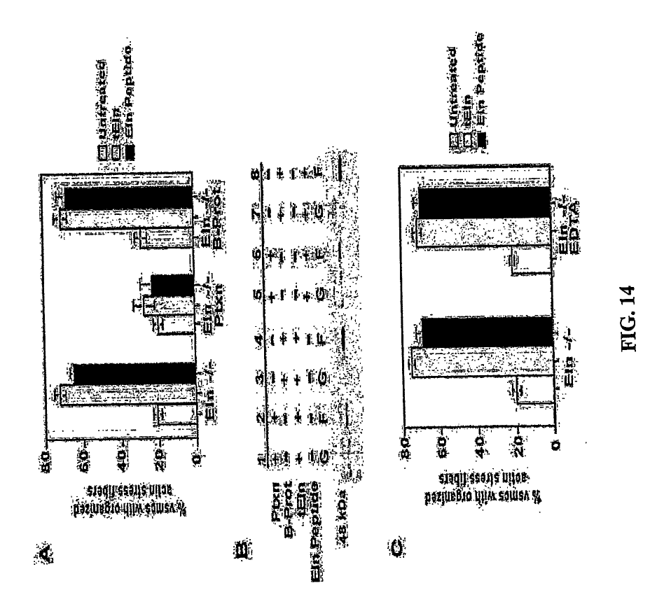

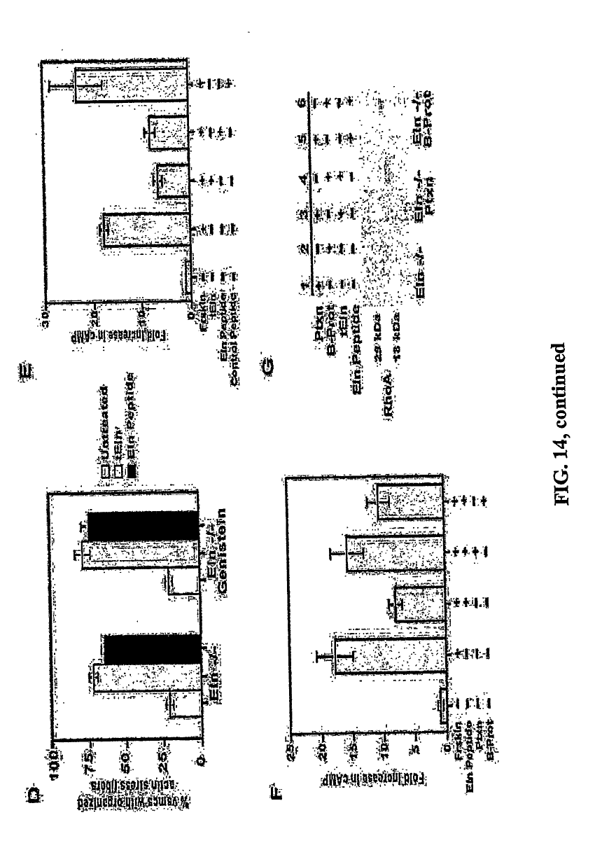

We performed a series of experiments to further demonstrate that heterotrimeric Gi proteins mediate stress fiber formation induced by both tropoelastin (SEQ ID NO: 2) and elastin peptide (SEQ ID NO: 3). FIGS. 14A and 14B show that the effect of tropoelastin and elastin peptide on actin stress fiber formation and the F:G actin ratio is significantly decreased if cells were pretreated with the Gi inhibitor pertussis toxin. Pretreatment of elastin −/− cells with 100 ng/ml of the heterotrimeric Gi protein inhibitor pertussis toxin significantly decreased the effect of tropoelastin or elastin peptide on myofilament organization and on the F:G actin ratio, however pretreatment with 100 ng/ml of the β protomer (inactivate form of pertussis toxin) did not have such an effect.

These results further support a model wherein elastin signals through a G-protein coupled receptor, a heterotrimeric Gi protein, and a RhoGTPase in order to influence vascular smooth muscle cell architecture and behavior. However, many proteins which signal through G-protein coupled receptors also signal through either receptor tyrosine kinases or integrin receptors. We performed a series of experiments to address whether elastin also signals through either an integrin receptor or a receptor tyrosine kinase, and FIGS. 14C and 14D summarize these results. Briefly, actin stress fiber formation in response to either tropoelastin (SEQ ID NO: 2) to elastin peptide (SEQ ID NO: 3) was assayed in the absence of pretreatment, following pretreatment with 20 μM EDTA, or following pretreatment with 10 μM genistein. Neither treatment with EDTA, an inhibitor of integrin signaling, nor treatment with genistein, a receptor tyrosine kinase inhibitor, altered the effect of tropoelastin or elastin peptide on vascular smooth muscle cells.

To further characterize elastin signaling via Gi proteins, we assessed the effect of tropoelastin and elastin peptide on cAMP levels. Given that Gi signaling is known to lower cAMP levels via inhibition of adenylate cyclase, elastin signaling via a Gi protein may have similar results on c AMP levels. FIGS. 14E and 14F summarize experiments designed to address this question. Briefly, vascular smooth muscle cells were pretreated with 10 μM forskolin to elevate basal cAMP levels. Following forskolin treatment, cells were either left untreated, or treated with 1 ug/ml tropoelastin (SEQ ID NO: 2), 10 μM elastin peptide (SEQ ID NO: 3), or 10 μM control peptide (SEQ ID NO: 4). Cells were then lysed and cAMP levels were obtained using a commercially available radioimmunoassay (RIA) kit (RPA538, Amersham Pharmacia). As shown in FIG. 14E, treatment of these cells with tropoelastin or elastin peptide, but not with control peptide, resulted in a decrease of forskolin induced cAMP levels. FIG. 14F shows that the effect of tropoelastin or elastin peptide on forskolin induced upregulation of cAMP levels is blocked by treatment with the Gi inhibitor pertussis toxin, but not by treatment with the β-protomer.

Finally, and to further confirm the results implicating both Gi proteins and RhoA GTPase in elastin signaling, we examined activation of RhoA in cells in response to tropoelastin or elastin peptide following pretreatment with pertussis toxin. FIG. 14E summarizes the results of these experiments which demonstrated that, in contrast to the activation of RhoA observed when vascular smooth muscle cells were treated with either tropoelastin (SEQ ID NO: 2) or elastin peptide (SEQ ID NO: 3), cells pretreated with pertussis toxin were unable to activate RhoA.

### Example 13

### Elastin Signaling Mediates Chemotaxis Through Heterotrimeric Gi Proteins

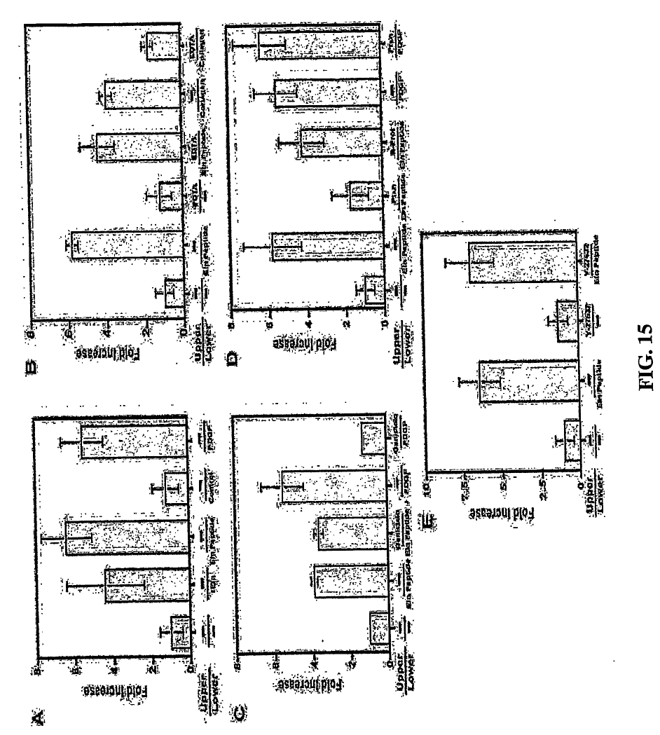

We have demonstrated that elastin signaling via Gi proteins and RhoA GTPase induces actin fiber formation. However, elastin signaling influences other aspects of vascular smooth muscle behavior including cell migration. Accordingly, we wanted to address whether elastin signaling influences other cellular behaviors via a similar mechanism involving activation of Gi proteins and RhoA GTPase. FIG. 15 summarizes a series of experiments examining the effects of elastin signaling on migration of vascular smooth muscle cells.

Briefly, chemotaxis was measured using a modified Boyden chamber method. Transwell polycarbonate filters were inserted into plastic 24-well tissue culture plates, and various concentrations of tropoelastin (SEQ ID NO: 2) or elastin peptide (SEQ ID NO: 3) were placed in both the upper and lower chambers which were separated by the filter. Prior to their addition to the upper chamber, vascular smooth muscle cells were incubated with either 20 μM EDTA (inhibitor of integrin receptors), 10 μM genistein (inhibitor of receptor tyrosine kinases), 100 ng/ml pertussis toxin, 100 ng/ml β-protomer, 20 μM Y27632, or the cells were left untreated. As a positive control, 30 ng/ml of PDGF-BB or 10 μg/ml of collagen type I, proteins known to promote cell migration, was added to the lower chamber of some of the wells. Cells were added to the upper chamber and incubated for three hours. Chemotactic response was measured as the number of cells to cross the filter separating the upper and lower chamber in response to tropoelastin, elastin peptide, PDGF, or collagen type I, and this number was expressed as fold increase over baseline.

FIG. 15 shows that elastin signaling mediated vascular smooth muscle cell chemotaxis through G-proteins. Specifically, FIG. 15A shows that elastin peptide (SEQ ID NO: 3) regulated vascular smooth muscle migration in a manner comparable to both tropoelastin and PDGF. FIGS. 15B and 15C show that the effect of elastin peptide was insensitive to treatment with either the integrin inhibitor EDTA or the tyrosine kinase inhibitor genistein. However, EDTA was able to block migration in response to the known integrin ligand collagen, and genistein was able to block migration in response to the receptor tyrosine kinase ligand PDGF. FIG. 15D shows that pertussis toxin, but not the inactive β-protomer, blocked the effects of elastin peptide on cell migration but had no impact on PDGF induced migration.

Finally, the results summarized in FIG. 15E suggest interesting mechanistic differences between the effect of elastin signaling on actin stress fiber formation and the effect of elastin signaling on chemotaxis. The Rho kinase inhibitor Y-27632 does not block the chemotactic activity of the elastin peptide suggesting a possible branch point in the elastin signaling pathway which mediates various affects of elastin signaling on cellular architecture and migration.

### Example 14

### Methods of Identifying the Elastin Receptor

Based on our understanding of elastin signaling, a screen to identify and/or characterize an elastin receptor can be performed. As demonstrated by FIG. 16, activation of elastin signaling in a cell which can respond to tropoelastin or a bioactive fragment thereof, and thus contains an elastin receptor, activates the potassium channel GiRK. Activation of GiRK therefore provides a read-out for elastin signaling, and provides a convenient assay upon which to base a screen for the elastin G-protein coupled receptor.

Briefly, a cell is used to express one or more candidate receptors. The cell is preferably one which is not typically responsive to elastin signaling. For example, an expression library is injected into an oocyte such as a Xenopus oocyte, and the oocyte is used to express the candidate receptors contained in the library. The library may be a library specifically enriched in G-protein coupled receptors, or a library which has not been previously enriched for G-protein coupled receptors. Additionally, the library can be a library specifically of G-protein coupled receptors.

In addition to the candidate receptor(s) (e.g., the library), GiRK 1 and GiRK 4 are also expressed in the same cell. In the present example, the oocyte is injected with RNA encoding GiRK 1 and GiRK 4. Expression of GiRK 1 and GiRK 4 is necessary to form functional GIRK channels in the cell. Such heteromultimeric channels can then be assayed for activity, and this activity serves as a read-out in the screen.

The oocyte expressing both functional GIRK channels and one or more candidate receptors is then contacted with tropoelastin or a bioactive fragment thereof, and the activity of the GIRK channel is measured in the presence of tropoelastin or a bioactive fragment thereof. This activity can be compared to the activity of the GIRK channel in the absence of tropoelastin or a bioactive fragment thereof. In the present example, oocytes such as Xenopus oocytes are amenable to electrophysiological methodologies. Accordingly, GIRK channel activity can be readily measured using, for example, whole cell patch clamping techniques well known in the art.

Activation of GIRK activity in the presence versus the absence of tropoelastin or a bioactive fragment thereof indicates that the cell expresses an elastin receptor. If the elastin receptor is contained within a library, the library can be sub-divided, and sub-divided pools of the library can be evaluated using this method until a single protein is identified as an elastin receptor.

Similarly, activation of GiRK channel activity can be used as an assay in any other cell based screening method. For example, activation of GIRK channel activity can be used as a read-out in a cell-based screen to identify elastin activators including small molecule activators. Similarly, activation of GIRK channel activity can be used to screen candidate bioactive tropoelastin peptides, or peptidomimetics thereof, to identify those peptides which are capable of activating elastin signaling.

All publications, patents and patent applications are herein incorporated by reference in their entirety to the same extent as if each individual publication, patent or patent application was specifically and individually indicated to be incorporated by reference in its entirety.

### EQUIVALENTS

Those skilled in the art will recognize, or be able to ascertain using no more than routine experimentation, many equivalents to the specific embodiments of the invention described herein. Such equivalents are intended to be encompassed by the following claims.

### REFERENCES

Eisenberg et al. (1964) Familial supravalvular aortic stenosis. Am. J. Dis. Child. 108: 341-347.
Perou (1961) Congenital Suparavalvular Aortic Stenosis. Arch. Path. 71: 113-126.
Schmidt et al. (1969) Generalized arterial fibromuscular dysplasia and myocardial infarction in familial supravalvular aortic stenosis syndrome. J Pediatr. 74(4): 576-84.
O'Connor et al. (1985) Supravalvular aortic stenosis. Clinical and pathologic observations in six patients. Arch Pathol Lab Med. 109(2): 179-85.
Ewart et al. (1993) A human vascular disorder, supravalvular aortic stenosis, maps to chromosome 7. Proc. Natl. Acad. Sci., USA. 90: 3226-3230.
Olson et al. (1995) A 30 kb deletion within the elastin gene results in familial supravalvular aortic stenosis. Hum Mol Genet. 4(9): 1677-9.
Curran et al. (1993) The elastin gene is disrupted by a translocation associated with supravalvular aortic stenosis. Cell 73: 159-168.
Ewart et al. (1993) Hemizygosity at the elastin locus in a developmental disorder, Williams syndrome Nat Genet. 5: 11-16.
Li et al. (1997) Elastin point mutations cause an obstructive vascular disease, supravalvular aortic stenosis. Hum. Mol. Genet. 6: 1021-1028.
Rosenbloom et al. (1993). FASEB J. 7:1208.
Parks et al. (1993) The extracellular matrix. Advances in Molecular and Cell Biology 6:133-182.
Lohler et al. (1984) Embryonic lethal mutation in mouse collagen I gene causes rupture of blood vessels and is associated with erythropoietic and mesenchymal cell death. Cell 38: 597-607.
Dietz and Pyeritz. (1995) Mutations in the human gene for fibrillin-1 (FBN1) in the Marfan syndrome and related disorders. Hum. Mol. Genet. 4: 1799-1809.
Nakamura. et al. (2002) Fibullin-5/DANCE is essential for elastogenesis in vivo. Nature 415: 171-175.
Yanagisawa et al. (2002) Fibulin-5 is an elastin-binding protein essential for elastic fibre development in vivo. Nature 415: 168-71.
Arteaga-Solis et al. (2000) Elastic and collagenous networks in vascular disease. Cell. Struct. Funct. 25: 69-72.
Dietz and Mecham (2000) Mouse models of genetic diseases resulting from mutations in elastic fiber formation. Matrix Biol. 19: 481-486.
Li et al. (1998) Elastin is an essential determinant of arterial morphogenesis. Nature 393: 276-280.
Li et al. (1998) Novel arterial pathology in mice and humans hemizygous for elastin. J. Clin. Invest. 102: 1783-1787.
Raines and Ross. (1993) Smooth muscle cells and the pathogenesis of the lesions of atherosclerosis. Br. Heart J. 69: S30.
Owens. (1998) Molecular control of vascular smooth muscle cell differentiation. Acta Physiol. Scand. 164(4): 623-35.
Heart and Stroke Statistical Update (2000) American Heart Association.
Ross. The pathogenesis of atherosclerosis: a perspective for the 1990s. (1993) Nature 362(6423): 801-9.
Sims. (2000) The initiation of intimal thickening in human arteries. Pathology 32(3): 171-5.
Sims. (1985) Discontinuities in the internal elastic lamina: a comparison of coronary and internal mammary arteries. Artery 13(3): 12743.
Hinek et al. (1991) Impaired elastin fiber assembly related to reduced 67-kD elastin binding protein in fetal lamb ductus arteriosis and in cultured aortic smooth muscle cells treated with chondroitin sulfate. J. Clin. Invest. 88: 2083-2094.
Zaidi et al. (2000) Suppressed smooth muscle proliferation and inflammatory cell invasion after arterial injury in elafin-overexpressing mice. J. Clin. Invest. 105: 1687-1695.
Cowan et al. (1996) Elafin, a serine elastase inhibitor, attenuates post-cardiac transplant coronary arteriopathy and reduces myocardial necrosis in rabbits after heterotopic cardiac transplantation. J. Clin. Invest. 97: 2452-2468.
Hedin et al. (1988) Diverse effects of fibronectin and laminin on phenotypic properties of cultured arterial smooth muscle cells. J. Cell Biol. 107(1): 307-19.
Koyama et al. (1996) Fibrillar collagen inhibits arterial smooth muscle proliferation through regulation of Cdk2 inhibitors. Cell 87(6):1069-78.
Yamamoto et al. (1993) Type I collagen promotes modulation of cultured rabbit arterial smooth muscle cells from a contractile to a synthetic phenotype. Exp Cell Res. 204(1): 121-9.
Yamamoto et al. (1995) Changes in elastin-binding proteins during the phenotypic transition of rabbit arterial smooth muscle cells in primary culture. Exp Cell Res. 218(1): 339-45.
Thompson and Parks. (1996) Role of matrix metalloproteinases in abdominal aortic aneurysms. Ann N Y Acad. Sci. 18;800:157-74.
Thompson and Baxter. (1999) MMP inhibition in abdominal aortic aneurysms. Rationale for a prospective randomized clinical trial. Ann N Y Acad. Sci. 30;878:159-78.
Hinek et al. (2000) Decreased elastin deposition and high proliferation of fibroblasts from Costello Syndrome are related to function deficiency in the 67-kD elastin-binding protein. Am. J. Hum. Genet. 66: 859-872.
Jung et al. (1998) Tropoelastin and elastin degradation products promote proliferation of human astrocytoma cell lines. J. Neuropathol. Exp. Neurol. 57: 439-448.
Senior et al. (1980) Chemotactic Activity of elastin-derived peptides. J. Clin. Invest. 66: 859-862.
Senior et al. (1982) Chemotactic responses of fibroblasts to tropoelastin and elastin-derived peptides. J. Clin. Invest. 70: 614-618.
Senior et al. (1984) Val-Gly-Val-Ala-Pro-Gly, a repeating peptide in elastin, is chemotactic for fibroblasts and monocytes. J. Cell Biol. 99: 870-874.
Wrenn et al. (1988) Kinetics of receptor-mediated binding of tropoelastin to ligament fibroblasts. J. Biol. Chem. 263: 2280-2284.
Blood et al. (1988) Identification of a tumor cell receptor for VGVAPG, an elastin-derived chemotactic peptide. J. Cell Biol. 107: 1987-1993.
Hornebeck et al. (1986) Inducible adhesion of mesenchymal cells to elastic fibers: Elastonectin. Proc. Natl. Acad. Sci. USA 83: 5517-5520.
Rao et al. (1983) Isolation of a tumor cell laminin receptor. Biochem. and Biophys. Res. Commun. 111: 804-808.
Mecham et al. (1989) Elastin binds to a multifunctional 67-kilodalton peripheral membrane protein. Biochemistry 28: 3716-3722.
Mecham et al. (1989) The elastin receptor shows structural and functional similarities to the 67-kDa tumor cell laminin receptor. J. Biol. Chem. 264: 16652-16657.
Hinek et al. (1988) The elastin receptor: a galactoside-binding protein. Science 239: 1539-1541.
Mecham and Hinek. (1996) Non-integrin laminin receptors. In: The Laminins (P. Ekblom & R. Timpl, eds.) Harwood Academic Publishers pp. 159-183.
Hinek. (1996) Biological roles of the non-integrin elastin/laminin receptor. J. Biol. Chem. 377:471-480.
Yow et al. (1988) Increased mRNA expression of a laminin-bnding protein in human colon carcinoma: complete sequence of a full-length cDNA encoding the protein. Proc. Natl. Acad. Sci. USA 85: 6494-6398.
Hinek et al. (1993) The 67-kD elastin/laminin-binding protein is related to an enzymatically inactive, alternatively spliced form of β-galactosidase. J. Clin. Invest. 91: 1198-1205.
Privitera et al. (1998) The 67-kDa enzymatically inactive alternatively spliced variant of β-galactosidase is identical to the elastin/laminin-binding protein. J. Biol. Chem. 273: 6319-6326.
Mecham et al. (1991) Ligand affinity of the 67-kD elastin/laminin binding protein is modulated by the protein's lectin domain: visualization of elastin/laminin-receptor complexes with gold-tagged ligands. J. Cell Biol. 113: 187-194.
Grosso et al (1991) Characterization of a putative clone for the 67-kilodalton elastin/laminin receptor suggests that it encodes a cytoplasmic protein rather than a cell surface receptor. Biochemistry 30: 3346-3350.
Casterella and Tierstein. (1997) Prevention of coronary restenosis. Cardiology in Review 7:219-231.
Rezai et al. (2001) Paclitaxel stent coating inhibits neointimal hyperplasia at 4 weeks in a porcine model of coronary restenosis. Circulation 103: 2289-2295.
Leon et al. (2001) Localization intracoronary gamma-radiation therapy to inhibit the recurrence of restenosis after stenting. N. Eng. J. Med. 344: 250-256.
Sousa et al. (2001) Sustained suppression of neointimal proliferation by sirolimus-eluting stents. Circulation 104: 2007-2011.
Gallo et al. (1999) Inhibition of intimal thickening after balloon angioplasty in porcine coronary arteries by targeting regulators of the cell cycle. Circulation 99: 2164-2170.
Teirstein (2001) Living the dream of no restenosis. Circulation. 104: 1996-1998. De Scheerder et al. (1995) Biocompatibility of polymer-coated oversized metallic stents implanted in normal porcine coronary arteries. Atherosclerosis, 114(1): 105-14.
Goodwin et al. (2000) Percutaneous delivery of a heparin-impregnated collagen stent-graft in aporcine model of atherosclerotic disease. Invest Radiology 35(7):420-5.
Small and Gimona. Acta. Physiol. Scand. 164: 341.
Burridge et al. (1988) Focal adhesion: Transmembrane junctions between the extracellular matrix and the cytoskeleton Ann. Rev. Cell Biol. 4: 487-525.
Dransfield et al. (1992) Divalent cation regulation of the function of the leukocyte integrin LFA-1. J Cell Biol 116:219-26
Howard et al. (2001) Orphan G-protein-coupled receptors and natural ligand discovery. Trends Pharmacol Sci. 22(3): 13240.
Malbon and Morris. (1999) Physiological regulation of G-protein-linked signaling. Physiol Rev 79: 1373-1430.
Medina et al. (2000) A switch mechanism for G beta gamma activation of I(KACh). J Biol Chem. 275(38): 29709-16.
Clapham. (1999) Unlocking family secrets: K+ channel transmembrane domains. Cell 97(5): 547-50.
Reusch et al. (2001) Gbeta/gamma mediate differentiation of vascular smooth muscle cells Journal of Biological Chemistry 276: 19540-19547
Malone et al. (1984) Detergent-extracted small-diameter vascular prosthesis. J. Vasc. Surg. 1: 181-91.
Ushiki and Murakumo. (1991) Scanning electron microscopic studies of tissue elastin components exposed by a KOH-Collagenase or simple KOH digestion method. Arch. Histol. Cytol. 54(4): 427-436.
Starcher. (1977) Determination of the elastin content of tissues by measuring desmosine and isodesmosine. Anal Biochem 1;79(1-2): 11-5.
Narayanaswamy et al. (2000) Vasc. Interv. Radiol. 11: 5.
Schwartz et al. (1994) Differential neointimal response to coronary artery injury in pigs and dogs. Arteriosclerosis and Thromb. 14(3): 395-400.
Schwartz et al. (1992) Restenosis and the proportional neointimal response to coronary artery injury: results in a porcine model. J. Am. Coll. Cardiol. 19: 267-274.
Schonthal et al. (1991) Transcriptional and post-transcriptional regulation of c-fos expression by the tumor promoter okadaic acid. Oncogene 6(3): 423-30.
Chang et al. (1995) Adenovirus-mediated over-expression of the cyclin/cyclin-dependent kinase inhibitor, p21 inhibits vascular smooth muscle cell proliferation and neointima formation in the rat carotid artery model of balloon angioplasty. J Clin Invest. 96(5): 2260-8.
Hengst and Reed. (1998) Inhibitors of the CIP/KIP family. Curr Top Microbiolo Immunol 227: 25-41.
Mack et al. (2001) Smooth Muscle Differentiation Marker Gene Expression Is Regulated by RhoA-mediated Actin Polymerization. J Biol Chem. 276(1): 341-347.
Coward et al. (1999) Chimeric G proteins allow a high-throughput signaling assay of Gi-coupled receptors. Anal Biochem. 270(2): 242-8.
Lim et al. (1992) Expression cloning of the TGF-β type II receptor, a functional transmembrane serine/threonine knase. Cell 68: 775-785.
Soker et al. (1998) Neuropilin-1 is expressed by endothelial and tumor cells as an isoform-specific receptor for vascular endothelial growth factor. Cell 92: 735-745.
Georges and Frech. (1992) Isolation of ion channel genes by expression cloning in xenopus oocytes. Methods of enzmmology. Vol 207 Ion Channels Editors: B. Rudy and L. Iverson. Academic Press New York
Birul et al. (1999) Reverse physiology in Drosophila: identification of a novel allatostatin-like neuropeptide and its cogante receptor structurally related to the mammalian somatostatin/galanin/opiod receptor family. EMBO 18: 5892-5900.
Goldin, A. L. (1991) Methods in Cell Biology 36, 487-509.
Sanguinetti, M. C., and Xu, Q. P. (1999) J. Physiol. 514: 667-675.
Stuhmer, W. (1992) Methods in Enzymology, Ion Channels 207, 319-339.
Caterina et al. (1997) The capsaicin receptor: a heat-activated ion channel in the pain pathway. Nature 389: 816-824.
Miller et al. (1999) Analysis of Apparent Noncompetitive Responses to Competitive H(1)-Histamine Receptor Antagonists in Fluorescent Imaging Plate Reader-Based Calcium Assays. J Biomol Screen 4(5): 249-258.
Grosso et al. (1991) Fibroblast adhesion to recombinant tropoelastin expressed as a protein A-fusion protein. Biochem. J 273: 517-522.
Urry et al. (1976) Synthetic, cross-linked polypentapeptide of elastin: an anisotropic, fibrillar elastomer. Biochemistry 15(18): 4083-4093.
Rapaka and Urry. (1978) Coacervation of sequential polypeptide models of elastin. Int. J. Peptide Protein Res. 1: 97-108.
Vrhovski et al. (1997) Coacervation characteristics of recombinant human elastin. Eur. J. Biochem. 250: 92-98.

1. A method of decreasing or preventing occlusion of a body vessel by smooth muscle cells, comprising administering an agent that promotes elastin signaling in an amount effective to decrease or prevent occlusion of a body vessel by smooth muscle cells.

2. A method of decreasing vascular obstruction, comprising administering an agent effective to decrease vascular obstruction, wherein said agent promotes elastin signaling thereby modulating vascular smooth muscle cells sufficiently to decrease vascular obstruction.

3. A method of promoting actin stress fiber formation in a smooth muscle cell, comprising contacting said smooth muscle cell with an agent that promotes elastin signaling in an amount effective to promote actin stress fiber formation.

4. A method of promoting actin polymerization in a smooth muscle cell, comprising contacting said smooth muscle cell with an agent that promotes elastin signaling in an amount effective to promote actin polymerization.

5. A method of increasing the ratio of F:G actin in a smooth muscle cell, comprising contacting said smooth muscle cell with an agent that promotes elastin signaling in an amount effective to increase the ratio of F:G actin.

6. A method for the treatment or prophylaxis of an obstructive vascular disease, comprising administering an agent that promotes elastin signaling in an amount effective to treat or prophylactically treat said obstructive vascular disease.

7. A method for preventing stenosis, comprising administering an amount of an agent effective to prevent stenosis, wherein said agent promotes elastin signaling.

8. A method for the treatment or prophylaxis of restenosis, comprising administering an amount of an agent effective to treat or prophylactically treat restenosis, wherein said agent promotes elastin signaling.

9. The method of claim 1, wherein said body vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

10. The method of claim 3, wherein said smooth muscle cell is a vascular smooth muscle cell.

11. The method of claim 1, further comprising administering a compound that inhibits proliferation of smooth muscle cells.

12. The method of claim 11, wherein said compound is selected from paclitaxel, rapamycin, actinomycin D, or radioactivity.

13. The method of claim 8, wherein said restenosis occurs following angioplasty.

14. The method of claim 1, wherein said agent is administered on a biocompatible device.

15. The method of claim 14, wherein said biocompatible device is an intraluminal device, and wherein said agent is administered to the site of occlusion or vascular obstruction.

16. The method of claim 15, wherein said intraluminal device is selected from a stent, a wire, a catheter, or a sheath.

17. The method of claim 1 or 2, wherein said agent is selected from a nucleic acid, peptide, polypeptide, peptidomimetic, small organic molecule, antisense oligonucleotide, RNAi construct, ribozyme, or antibody.

18. The method of claim 17, wherein said agent promotes elastin signaling by
(i) binding to and activating the elastin receptor,
(ii) increasing the expression and/or activity of tropoelastin,
(iii) increasing the expression and/or activity of a protein that activates the elastin receptor,
(iv) activating Gi, or
(v) activating RhoA.

19. The method of claim 17, wherein said agent promotes elastin signaling by inhibiting the expression and/or activity of a protein that represses elastin signaling.

20. The method of claim 19, wherein said protein that represses elastin signaling is a protein that promotes the off state of RhoA.

21. The method of claim 20, wherein said protein that promotes the off state of RhoA is a GAP or GDI.

22. (canceled)

23. The method of claim 17, wherein said agent is selected from elastin receptor, a constituitively active elastin receptor, Gi, a constituitively active Gi, RhoA, a constituitively active RhoA, or a GAP.

24. The method of claim 17, wherein said agent comprises a polypeptide comprising an amino acid sequence at least 80% identical to any of SEQ ID NO: 2, SEQ ID NO: 3, or SEQ ID NO: 6.

25. Use of an agent that promotes elastin signaling in smooth muscle cells in the manufacture of a medicament for the treatment or prevention of occlusion of a vessel.

26. The use of claim 25, wherein said smooth muscle cell is a vascular smooth muscle cell.

27. The use of claim 25, wherein said vessel is selected from any of artery, vein, common bile duct, pancreatic duct, kidney duct, esophagus, trachea, urethra, bladder, uterus, ovarian duct, Fallopian tube, vas deferens, prostatic duct, or lymphatic duct.

28. The use of claim 25, wherein said agent is selected from any of a nucleic acid, a peptide, a polypeptide, a peptidomimetic, a small organic molecule, an antibody, an antisense oligonucleotide, an RNAi construct, or a ribozyme.

29. The method of claim 1, wherein said agent is formulated in a pharmaceutically acceptable carrier.

30. A method of screening to identify and/or characterize an elastin activator that promotes elastin signaling in smooth muscle cells, comprising
(a) contacting a cell with one or more agents;
(b) comparing elastin signaling in said cell in the presence of said one or
more agents in comparison to the absence of said one or more agents, wherein an agent that promotes elastin signaling in a cell is an elastin activator.

31. The method of claim 30, further comprising formulating said agent that promotes elastin signaling in a pharmaceutically acceptable carrier.

32. An agent identified by the method of claim 30, wherein said agent has any of the following functions:
(i) increases actin stress fiber formation in smooth muscle cells,
(ii) increases expression of vinculin in smooth muscle cells,
(iii) increases focal adhesion formation in smooth muscle cells,
(iv) inhibits dedifferentiation of smooth muscle cells,
(v) promotes actin polymerization in smooth muscle cells,
(vi) increases the ratio of F:G actin in smooth muscle cells,
(vii) decreases or inhibits occlusion of a vessel,
(viii) decreases or inhibits vascular obstruction,
(ix) decreases or inhibits restenosis, or
(x) prevents stenosis.

33. The agent of claim 32 formulated in a pharmaceutically acceptable carrier.

34. The method of claim 30, wherein screening of one or more agents comprises screening a library of agents.

35. The method of claim 34, wherein said one or more agents is a nucleic acid, peptide, polypeptide, small organic molecule, antibody, antisense oligonucleotide, ribozyme, or RNAi construct.

36. A method of conducting a drug discovery business comprising:
(a) identifying, by the assay of claim 30, one or more agents which promote elastin signaling;
(b) conducting therapeutic profiling of an agent identified for efficacy and toxicity in one or more animal models; and
(c) formulating a pharmaceutical preparation including one or more agents identified as having an acceptable therapeutic profile.

37. The method of claim 36, further including establishing a system for distributing the pharmaceutical preparation for sale.

38. The method of claim 36, further including establishing a sales group for marketing the pharmaceutical preparation.

39. A method of screening to identify and/or characterize an elastin receptor, comprising
(a) expressing at least one candidate receptor in a cell which is not responsive to elastin signaling;
(b) expressing GiRK 1 and GiRK 4 in said same cell, whereby said cell expresses a heteromultimeric GiRK channel;
(c) contacting said cell with tropoelastin or a bioactive fragment thereof,
(d) measuring activity of said heteromultimeric GiRK channel in the presence of tropoelastin or a bioactive fragment thereof in comparison to the activity of said heteromultimeric GiRK channel in the absence of tropoelastin or a bioactive fragment thereof;
wherein activation of said heteromultimeric GiRK channel in the presence of tropoelastin or a bioactive fragment thereof indicates that the candidate receptor is an elastin receptor.

40. The method of claim 39, wherein said cell is an oocyte.

41. The method of claim 40, wherein said oocyte is a Xenopus oocyte.

42. The method of claim 39, wherein said at least one candidate receptor is a library of candidate receptors.

43. The method of claim 42, wherein said library of candidate receptors is a library of G-protein coupled receptors.

44. The method of claim 39, wherein activation of said heteromultimeric GiRK channel is measured using whole cell clamping.


SEQUENCE LISTING (6 sequences)

SEQ ID NO: 1 - DNA, 2260 bp, Homo sapiens
ctcgagaagagagggctccagctccccacagtagcccctgccttcctccttcccaggcaggcggaggcacgcagatataccattgacttcccctcccctgcagcaggcacatcctgggcatcgagcttcagaccctgcccctgagcagcccctaaccccaccaacaaagggtggcttgggggggctttcaccccagcataatctccatcagctaccctcaaagcacccccaaataaacacacaccgtaagtaagagctgtacactggctgtgtgcgtacatcttcaagacaattctcccagcatgcccctaccttccaaaattccagagctgctccctccaaagacccagggaaaaggaagggtttgtccagggtcctggggtggccccgtatagaccaaagcctgataactgtcctagaagcagagtacttgcagagcgagtgacggcaactgtggtattgacaccagtcctagcaccagctgaacacagagcatttttgatctagcagaaatacaagaccacgttgtatttgtctttgcaataatctcttagctaggaatactgatcacctgtagacagataaggaaactgatgctctgtggagaggttttcctaccagaaaggctagagccagaaatttacttctaggtccaccaatacctgcctttgaccaatgcctgcatttgacctttccacgctgagccacccctgctggcactccagactgccacagtgctcctgcctccacaaggggtctttaactcatccctcggagccatcgtggtgcagggaaaagcccacagggcgtgtggcttccatgctgttccctgactggctgtgacctaggacaaggaacaagtttccctctcctattctctaggtctcacatttcttctcctctagcagtagtgggaagtgaggggtgggggacacgaccctcccctgttccatcccacactccaacccccaaaatcccccagggtccccgtccagctcagtcctgggggcagaaatgcagagttctccaggaacgtggtcccagctgtttcagtgcaggccgccccctcctggccaccagcggaatgtcagccttcccagaggggccgggagaacagcagtcgagaagctcccagactggtgtgggcgctagctgtgctcagcgtggggatgggaggtgacccagtgataatgggaagctgggctgcctgtcagtctgtggggggctcccacctccctgttcccccacagggcacctggggatccagcctgatttttaccagacctgcggcctgcatggggctgggtatagggctgtgaccttgacccatgcagaatagaaccctgtgtgtcgggatcctccatgtgctccagatgcccctggggacagcaccaacatggccttaactcccaagccattcccctgcctctaaccccctggcatctgcaggcatccaccccagacccacccaacacctcctccccagcttcaggcgctaggcagagaccttggcccctgcagaatgcagccctgtccagggtcccctaccttccccccagatccctcccagagcaataccaacccgggcctaccttccaggccattcaacctgcagccccccggcctctgtagacatcgcaccccccaaacccccagacctgcccaatgcctcccctccccagctttgggcagaacctgtctctagccagacctgggggtgttggggagtctggagggccggggtgggggctgaggcgcgggacagctggcccgtatcctcacactgggcccagccggaggggcgggggcctggccactcgggccttggctggggctgggatttttggcctggccgccaggccctcccttctgcttcctctcccgagggctgtcctggcagaggcccccctcgctctttctggcgggaacagggccagcagcgaaagaacagtcgcagagggaaagcgggaaagagatgggggaaagtgtgtgtgtgtgagtgtgtgcttgtgtgcatgtgtgtgcgtgtgttgtgtcaagaaaaaagctcgcagtccagcagcccgggcctgggaggcttgtgagccgggcctttcgtaattgtcccctccccgcggccccctcccccaggcctcccccctctcccgccctcccgcccgccctctctccctccctctttccctcacagccgacgaggcaacaattaggctttggggataaaacgaggtgcggagagcgggctggggcatttctccccgag

SEQ ID NO: 2 - protein, 757 aa, Homo sapiens
MAGLTAAAPRPGVLLLLLSILHPSRPGGVPGAIPGGVPGGVFYPGAGLGALGGGALGPGGKPLKPVPGGLAGAGLGAGLGAFPAVTFPGALVPGGVADAAAAYKAAKAGAGLGGVPGVGGLGVSAGAVVPQPGAGVKPGKVPGVGLPGVYPGGVLPGARFPGVGVLPGVPTGAGVKPKAPGVGGAFAGIPGVGPFGGPQPGVPLGYPIKAPKLPGGYGLPYTTGKLPYGYGPGGVAGAAGKAGYPTGTGVGPQAAAAAAAKAAAKFGAGAAGVLPGVGGAGVPGVPGAIPGIGGIAGVGTPAAAAAAAAAAKAAKYGAAAGLVPGGPGFGPGVVGVPGAGVPGVGVPGAGIPVVPGAGIPGAAVPGVVSPEAAAKAAAKAAKYGARPGVGVGGIPTYGVGAGGFPGFGVGVGGIPGVAGVPSVGGVPGVGGVPGVGISPEAQAAAAAKAAKYGVGTPAAAAAKAAAKAAQFGLVPGVGVAPGVGVAPGVGVAPGVGLAPGVGVAPGVGVAPGVGVAPGIGPGGVAAAAKSAAKVAAKAQLRAAAGLGAGIPGLGVGVGVPGLGVGAGVPGLGVGAGVPGFGAGADEGVRRSLSPELREGDPSSSQHLPSTPSSPRVPGALAAAKAAKYGAAVPGVLGGLGALGGVGIPGGVVGAGPAAAAAAAKAAAKAAQFGLVGAAGLGGLGVGGLGVPGVGGLGGIPPAAAAKAAKYGAAGLGGVLGGAGQFPLGGVAARPGFGLSPIFPGGACLGKACGRKRK

SEQ ID NO: 3 - protein, 6 aa, Artificial Sequence
  A bioactive fragment of tropoelastin.
VGVAPG

SEQ ID NO: 4 - protein, 6 aa, Artificial Sequence
  Control random fragment.
VSLSPG

SEQ ID NO: 5 - DNA, 582 bp, Homo sapiens
atggctgccatccggaagaaactggtgattgttggtgatggagcctgtggaaagacatgcttgctcatagtcttcagcaaggaccagttcccagaggtgtatgtgcccacagtgtttgagaactatgtggcagatatcgaggtggatggaaagcaggtagagttggctttgtgggacacagctgggcaggaagattatgatcgcctgaggcccctctcctacccagataccgatgttatactgatgtgtttttccatcgacagccctgatagtttagaaaacatcccagaaaagtggaccccagaagtcaagcatttctgtcccaacgtgcccatcatcctggttgggaataagaaggatcttcggaatgatgagcacacaaggcgggagctagccaagatgaagcaggagccggtgaaacctgaagaaggcagagatatggcaaacaggattggcgcttttgggtacatggagtgttcagcaaagaccaaagatggagtgagagaggtttttgaaatggctacgagagctgctctgcaagctagacgtgggaagaaaaaatctggttgccttgtcttgtga

SEQ ID NO: 6 - protein, 193 aa, Homo sapiens
MAAIRKKLVIVGDGACGKTCLLIVFSKDQFPEVYVPTVFENYVADIEVDGKQVELALWDTAGQEDYDRLRPLSYPDTDVILMCFSIDSPDSLENIPEKWTPEVKHFCPNVPIILVGNKKDLRNDEHTRRELAKMKQEPVKPEEGRDMANRIGAFGYMECSAKTKDGVREVFEMATRAALQARRGKKKSGCLVL